SEQUENCE LISTING (35 sequences)

SEQ ID NO: 1 - protein, 108 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 2 - DNA, 20 bp, synthetic construct
  source 1..20: mol_type = other DNA
tacgttctctatcactgata

SEQ ID NO: 3 - DNA, 23 bp, synthetic construct
  source 1..23: mol_type = other DNA
gcagacgcgaggaaggagggcgc

SEQ ID NO: 4 - DNA, 20 bp, synthetic construct
  source 1..20: mol_type = other DNA
agccggacaggacctcccag

SEQ ID NO: 5 - DNA, 22 bp, synthetic construct
  source 1..22: mol_type = other DNA
gaccctgctgtttgcgggtggt

SEQ ID NO: 6 - DNA, 19 bp, synthetic construct
  source 1..19: mol_type = other DNA
gagggagaaggcggggtgg

SEQ ID NO: 7 - DNA, 20 bp, synthetic construct
  source 1..20: mol_type = other DNA
gtgtactctctgaggtgctc

SEQ ID NO: 8 - DNA, 20 bp, synthetic construct
  source 1..20: mol_type = other DNA
gacgcagataagaaccagtt

SEQ ID NO: 9 - DNA, 20 bp, synthetic construct
  source 1..20: mol_type = other DNA
gcatcaagtcagccatcagc

SEQ ID NO: 10 - DNA, 21 bp, synthetic construct
  source 1..21: mol_type = other DNA
gcgggagaaaggaacgggagg

SEQ ID NO: 11 - DNA, 21 bp, synthetic construct
  source 1..21: mol_type = other DNA
gtccaatttctagggtcaccg

SEQ ID NO: 12 - DNA, 21 bp, synthetic construct
  source 1..21: mol_type = other DNA
gaagaacttgaagcaaagcgc

SEQ ID NO: 13 - DNA, 19 bp, synthetic construct
  source 1..19: mol_type = other DNA
cctgggctccggggcgttt

SEQ ID NO: 14 - DNA, 19 bp, synthetic construct
  source 1..19: mol_type = other DNA
ggcccctgcggccaccccg

SEQ ID NO: 15 - DNA, 19 bp, synthetic construct
  source 1..19: mol_type = other DNA
ctccctccctgcccggtag

SEQ ID NO: 16 - DNA, 19 bp, synthetic construct
  source 1..19: mol_type = other DNA
aggtttggaaagggcgtgc

SEQ ID NO: 17 - DNA, 20 bp, synthetic construct
  source 1..20: mol_type = other DNA
gcgaggtattcggctccgcg

SEQ ID NO: 18 - DNA, 21 bp, synthetic construct
  source 1..21: mol_type = other DNA
actacaccgaggaaatgggct

SEQ ID NO: 19 - DNA, 22 bp, synthetic construct
  source 1..22: mol_type = other DNA
cccacaatgccagttaagaaga

SEQ ID NO: 20 - DNA, 20 bp, synthetic construct
  source 1..20: mol_type = other DNA
ctctctgctcctttgccaca

SEQ ID NO: 21 - DNA, 20 bp, synthetic construct
  source 1..20: mol_type = other DNA
gtgctcttcgggtttcagga

SEQ ID NO: 22 - DNA, 20 bp, synthetic construct
  source 1..20: mol_type = other DNA
caatgaccccttcattgacc

SEQ ID NO: 23 - DNA, 20 bp, synthetic construct
  source 1..20: mol_type = other DNA
ttgattttggagggatctcg

SEQ ID NO: 24 - DNA, 20 bp, synthetic construct
  source 1..20: mol_type = other DNA
ggaatccatggagggaagat

SEQ ID NO: 25 - DNA, 20 bp, synthetic construct
  source 1..20: mol_type = other DNA
tgttctcgctcaggtcagtg

SEQ ID NO: 26 - DNA, 19 bp, synthetic construct
  source 1..19: mol_type = other DNA
cgcggccaacaagaagatg

SEQ ID NO: 27 - DNA, 20 bp, synthetic construct
  source 1..20: mol_type = other DNA
cgacgagtaggatgagaccg

SEQ ID NO: 28 - DNA, 20 bp, synthetic construct
  source 1..20: mol_type = other DNA
ctgtggcgcgtccatccttg

SEQ ID NO: 29 - DNA, 20 bp, synthetic construct
  source 1..20: mol_type = other DNA
tggcgttggaagcttggccc

SEQ ID NO: 30 - DNA, 20 bp, synthetic construct
  source 1..20: mol_type = other DNA
tctgcccgctcggagagggg

SEQ ID NO: 31 - DNA, 22 bp, synthetic construct
  source 1..22: mol_type = other DNA
gcctgagtgctccagtagccac

SEQ ID NO: 32 - DNA, 20 bp, synthetic construct
  source 1..20: mol_type = other DNA
catccccgcttccccaagga

SEQ ID NO: 33 - DNA, 20 bp, synthetic construct
  source 1..20: mol_type = other DNA
gtgtctcccccttgagtccc

SEQ ID NO: 34 - DNA, 1128 bp, synthetic construct
  source 1..1128: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcgggatccgcgggggtccaagttgaaaccattagtcccggcgatggcaggacttttcctaaaaggggacaaacatgtgtcgtccattatacaggcatgttggaggacggcaaaaaggtcgacagtagtagagatcggaataaacctttcaaattcatgttgggaaaacaagaagtcattaggggatgggaggagggcgtggctcaaatgtccgtcggccaacgggctaagctcaccatcagccccgactacgcttacggcgctaccggccatcccggaattatcccgccccacgctaccttggtgtttgacgtcgaattgttgaagctcgaactcgagggcgtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatag

SEQ ID NO: 35 - DNA, 12 bp, synthetic construct
  source 1..12: mol_type = other DNA
gatcggaagagc

(19) United States
(12) Patent Application Publication
(10) Pub. No.: US 2025/0177566 A1
(43) Pub. Date: Jun. 5, 2025

(54) USE OF CHEMICAL EPIGENETIC MODIFIERS TO MODULATE GENE EXPRESSION

(71) Applicant: The University of North Carolina at Chapel Hill, Chapel Hill, NC (US)

(72) Inventors: Nathaniel A. Hathaway, Chapel Hill, NC (US); Lindsey Ingerman James, Chapel Hill, NC (US); Dongbo Lu, Chapel Hill, NC (US); Caroline Foley, Carrboro, NC (US); Sara Wasserman, Cary, NC (US)

(21) Appl. No.: 18/843,505

(22) PCT Filed: Mar. 7, 2023
(86) PCT No.: PCT/US2023/063818

§ 371 Date: Sep. 3, 2024

Related U.S. Application Data
(60) Provisional application No. 63/317,373, filed on Mar. 7, 2022.
(60) Provisional application No. 63/320,910, filed on Mar. 17, 2022.

(51) Int. Cl. A61K 48/00; A61K 31/44; A61K 31/5517; C07D 211/60; C07D 487/04; C07D 495/14; C12N 9/22; C12N 9/90; C12N 15/11; C12N 15/85

(52) U.S. Cl. CPC A61K 48/005; A61K 31/44; A61K 31/5517; C07D 211/60; C07D 487/04; C07D 495/14; C12N 9/22; C12N 9/90; C12N 15/111; C12N 15/85; C07K 2319/80; C12N 2310/20; C12Y 502/01008

(57) ABSTRACT

The invention relates to relates to a chemical epigenetic modifier (CEM) comprising compound 1 (AP1867) or a pharmaceutically acceptable salt thereof, a linker, and a chromatin regulatory protein ligand and compositions thereof. The invention also relates to methods for modulating expression of a gene comprising contacting a polynucleotide with a target gene sequence to a fusion protein comprising a FK506-binding protein (FKBP) polypeptide with a F36V mutation and a gRNA binding polypeptide, a gRNA comprising a gene targeting polynucleotide sequence that binds to a target gene sequence and a polynucleotide sequence recognized by the gRNA binding polypeptide, a CEM, and a protein with a DNA binding domain that binds to the target gene sequence and the gRNA, thereby modulating expression of the gene.

### STATEMENT OF PRIORITY

This application claims the benefit, under 35 U.S.C. § 119 (e), of U.S. Provisional Application No. 63/317,373, filed Mar. 7, 2022, and U.S. Provisional Application No. 63/320,910, filed Mar. 17, 2022, the entire contents of each of which is incorporated by reference herein in its entirety.

### GOVERNMENT SUPPORT

This invention was made with government support under Grant Number GM118653, awarded by the National Institutes of Health. The government has certain rights in this invention.

### STATEMENT REGARDING ELECTRONIC FILING OF A SEQUENCE LISTING

A Sequence Listing in XML format, entitled 5470-926WO_ST26.xml, 36,864 bytes in size, generated on Mar. 5, 2023 and filed herewith, is hereby incorporated by reference in its entirety for its disclosures.

### FIELD OF THE INVENTION

This invention relates to methods and compositions for modulating gene expression, e.g., for gene therapy. In particular, the invention relates to chemical epigenetic modifiers (CEMs), their compositions, and methods for regulating gene expression.

### BACKGROUND OF THE INVENTION

Proper epigenomic regulation is necessary for eukaryotic cells to function properly, and dysregulation of chromatin is often closely linked to disease. Epigenetic post-translational modifications on both DNA and histone tails can influence the varying degrees of chromatin compaction which contribute to multiple biological functions. Epigenetic marks can be added, removed, or read by various endogenous chromatin machinery that control gene expression simultaneously. Lysine acetylation on histone tails is one example of post-translational modification (PTM) that plays an important role in chromatin compaction and gene expression. The pair of protein families responsible for the regulation of histone acetylation are histone acetyl transferases (HATs) and histone deacetylases (HDACs). Researchers have developed and engineered various technologies using chromatin landscape sculpturing pathways to regulate gene expression in order to both study biological mechanisms and help treat diseases, such as cancers.

With the rapid development of CRISPR technology, the study and control of chromatin compaction has advanced significantly. Multiple research labs have utilized CRISPR-Cas9 systems and exogenous chromatin regulatory proteins to manipulate gene expression in a locus-specific manner. Some of the gene-specific regulation approaches have engineered deactivated Cas9 (dCas9) fusion proteins such as dCas9-p300, dCas9-KRAB, and dCas9-VPR. Other technologies use chemicals or light to induce heterodimerizations of dCas9 and protein regulators to facilitate gene regulation in a more temporal manner. In addition to CRISPR-Cas9, other chromatin engaging technologies including Zinc Fingers (ZFs), transcription activator-like effectors (TALEs), and polyamides have been exploited for gene regulation purposes.

Biological technologies involving endogenous protein complexes have also emerged in recent years. Proteolysis targeting chimeras (PROTACs) are bifunctional molecules composed of ligands for both an endogenous E3 ligase and a protein-of-interest (POI). By inducing a unique E3:POI protein complex, PROTACs promote E3-mediated ubiquitination and subsequent proteasomal degradation of the POI. For example, the well-known PROTAC molecules, dBET1, ARV-771, and MZ1, are all comprised of the BET bromodomain ligand JQ1 and have demonstrated potent BRD4 degradation. Nonetheless, there are few technologies that employ endogenous chromatin sculpturing machinery for gene-specific regulation using small bifunctional molecules. The work by Liszczak et al demonstrated recruitment of endogenous BRD4 or the PCR1 complex by incorporating a dCas9-IntN and IntC-fused multimeric JQ1 construct or IntC-fused UNC3866 molecule, respectively, to regulate gene expression.

There is a need in the art for epigenetic modifiers to regulate gene expression, particularly with increased precision.

### SUMMARY OF THE INVENTION

The present invention is based on the discovery that gene expression can be modulated by recruiting epigenetic modifiers to the gene. Using these epigenetic modifiers, gene regulation can be more precisely regulated to produce increased amounts of the gene product when needed and to decrease expression when needed, thereby providing maximum benefits for gene therapy and other uses while minimizing toxicity.

Previous work by the inventors has demonstrated the specific activation and repression of chromosomal gene expression via the recruitment of modifiers that generate euchromatin or heterochromatin, respectively (Hathaway et al., Cell 149 (7): 1447 (2012); Vignaux et al., PLOS One 14 (7): e0217699 (2019)). This strategy is reliant on a fusion protein consisting of a targeted zinc finger (ZF) DNA binding domain to a host domain, FKBP, that interacts with bi-functional Chemical Epigenetic Modifiers (CEMs) (Butler et al., ACS Synth. Biol. 7 (1): 38 (2018)). Following reports using this system in specific chromosomal gene activation or repression (Chiarella et al., J. Vis. Exp. 2018 (139); Gryder et al., Nat. Genet. 51 (12): 1714 (2019); Chiarella et al., Nat. Biotechnol. 38 (1): 50 (2020)), extrapolated proof-of-concept studies regulating transduced AAV vector episomes have generated data in human cells demonstrating the ability to control AAV transgene expression via recruitment of chromatin modifiers. Specifically, individual recruitment of BRD4 to AAV episomes resulted in the rapid and significant enhancement of transgene expression. These novel observations in well controlled and rigorous experiments demonstrate that AAV episomes are naturally restricted for expression in human cells and allude to the ability to specifically induce therapeutic transgene expression at a fixed AAV vector dose.

Thus, one aspect of the invention relates to a chemical epigenetic modifier (CEM) comprising compound 1 (AP1867) or a pharmaceutically acceptable salt thereof, a linker, and a chromatin regulatory protein ligand. In one embodiment, the CEM has the structure of Formula I:

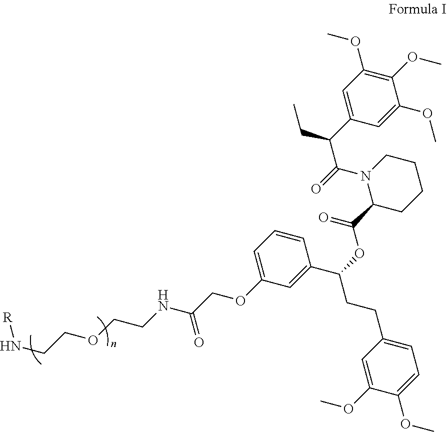

wherein n is 1-10 or 2-7; and
wherein R is a chromatin regulatory protein ligand. In some embodiments, R is a bromodomain and extraterminal (BET) protein ligand or a histone deacetylase (HDAC) protein ligand. In one embodiment, R is a BRD4 or CBP/300 ligand.

Another aspect of the invention is a method of modulating expression of a target gene, the method comprising contacting the target gene with: 1) a fusion protein comprising a FK506-binding protein (FKBP) polypeptide with a F36V mutation that binds a CEM and a gRNA binding polypeptide that binds gRNA; 2) a gRNA comprising a gene targeting polynucleotide sequence that binds to a target gene sequence and polynucleotide sequence recognized by the gRNA binding polypeptide; 3) a protein with a DNA binding domain that binds to the target gene sequence and the gRNA gene targeting polynucleotide sequence; and 4) the CEM of the present invention to thereby modulate expression of the target gene. In an aspect, the fusion protein binds the CEM and the gRNA, the gRNA forms a complex with the protein with the DNA binding domain binding the target gene, and the CEM further binds the chromatin regulatory protein, bringing the chromatin regulatory protein in proximity to the target gene to thereby modulate expression of the target gene.

Another aspect of the invention is a method of modulating expression of a target gene in a subject, the method comprising administering to the subject: 1) a fusion protein comprising a FK506-binding protein (FKBP) polypeptide with a F36V mutation that binds a CEM and a gRNA binding polypeptide that binds gRNA; 2) a gRNA comprising a gene targeting polynucleotide sequence that binds to a target gene sequence and polynucleotide sequence recognized by the gRNA binding polypeptide; 3) a protein with a DNA binding domain that binds to the target gene sequence and the gRNA gene targeting polynucleotide sequence; and 4) the CEM of the present invention to thereby modulate expression of the target gene.

A further aspect of the invention is a method of treating a disorder that is treatable by modulating expression of a gene in a subject in need thereof comprising of a target gene in a subject, the method comprising administering to the subject: 1) a fusion protein comprising a FK506-binding protein (FKBP) polypeptide with a F36V mutation that binds a CEM and a gRNA binding polypeptide that binds gRNA; 2) a gRNA comprising a gene targeting polynucleotide sequence that binds to a target gene sequence and polynucleotide sequence recognized by (capable of binding) the gRNA binding polypeptide; 3) a protein with a DNA binding domain that binds to the target gene sequence and the gRNA gene targeting polynucleotide sequence; and 4) the CEM of the present invention to modulate expression of the target gene to thereby treat the disorder. In an aspect, the fusion protein binds the CEM and the gRNA, the gRNA forms a complex with the protein with the DNA binding domain binding the target gene, and the CEM further binds the chromatin regulatory protein, bringing the chromatin regulatory protein in proximity to the target gene to modulate the gene and thereby treat the disorder.

Another aspect of the invention comprises a method of modulating expression of a transgene from a transgene delivery vector, the method comprising providing a transgene delivery vector comprising a polynucleotide comprising a transgene expression cassette and a nucleic acid binding domain recognition sequence; contacting the transgene delivery vector with a fusion protein, the fusion protein comprising a nucleic acid binding domain that binds to the recognition sequence fused to a domain that binds a CEM of the present invention; and contacting the transgene delivery vector with the chemical epigenetic modifier; thereby modulating expression of the transgene from the transgene delivery vector.

A further aspect of the present invention comprises a method of modulating expression of a transgene from a transgene delivery vector in a subject, the method comprising administering to the subject a transgene delivery vector comprising a polynucleotide comprising a transgene expression cassette and a nucleic acid binding domain recognition sequence; administering to the subject a fusion protein, the fusion protein comprising a nucleic acid binding domain that binds to the recognition sequence fused to a domain that binds a CEM; and administering to the subject the chemical epigenetic modifier; thereby modulating expression of the transgene.

A further aspect of the present invention comprises a method of treating a disorder that is treatable by expression of a transgene from a transgene delivery vector in a subject in need thereof, the method comprising: administering to the subject a transgene delivery vector comprising a polynucleotide comprising a transgene expression cassette and a nucleic acid binding domain recognition sequence; administering to the subject a fusion protein, the fusion protein comprising a nucleic acid binding domain that binds to the recognition sequence fused to a domain that binds a CEM; and administering to the subject the chemical epigenetic modifier; thereby treating the disorder.

These and other aspects of the invention are set forth in more detail in the description of the invention below.

### BRIEF DESCRIPTION OF THE DRAWINGS

FIGS. 1A-1E show dCas9-based SLF-CEM activating technology and the GFP activation in HCT116 cells. FIG. 1A is a demonstration that dCas9-based bumped synthetic ligand of FKBP* (SLF*)-CEMa, dCas9, MS2 stemloop contained gRNA and MS2-FKBP*x2 can recognize and bind a targeted DNA sequence. The small bifunctional molecule SLF*-CEMa can bind to FKBP* via AP1867, and recruit BRD4 by the recruitment warhead. Endogenous activating machinery will be located at the targeted gene locus and activate gene expression. FIG. 1B is a table of SLF*-CEMa molecules, showing two recruitment warheads were used: (+)-JQ1 and I-BET762. Exemplary polyethylene glycol (PEG) linker lengths are from 2 to 7 monomeric units in length. FIG. 1C shows individual component of SLF*-CEMa control in a reporter gene activation experiment. HCT116 cells were transfected with TRE3G-GFP reporter gene, dCas9, and MS2-FKBP*x2. Either TRE3G-GFP targeting gRNA or no targeting gRNA was transfected. AP1867 alone, I-BET762-COOH alone, unlinked AP1867 and I-BET762-COOH, or CEM202 were added to a final concentration of 10 nM in cell media and the GFP activation in flow cytometry was demonstrated for each. FIG. 1D shows an AP1867 and CEM207 competition experiment. 5 nM CEM207 was used to treat HCT116 cells in the reporter gene experiment, along with 0 μM, 5 μM, 10 μM, or 20 μM AP1867. The GFP expression change was shown by flow cytometry. FIG. 1E is a dose curve showing CEM202, CEM207, and CEM87 in the SLF*-CEMa system (dCas9-HA ms2-FKBP*x2 with TRE3G gRNA). CEM87 is described in International Patent Publication WO 2022/236010, for example, at [0093], incorporated herein by reference in its entirety. HCT116 cells were treated with CEM202, CEM207, and CEM87 with concentrations ranging from 0.25 nM to 100 nM. The GFP expression was collected by flow cytometry. In FIGS. 1C-1D, significance was determined by three biological replicates using a two-tailed Student's t-test. P values and fold changes are reported in Table 2. Error bars represent the standard deviation.

FIGS. 2A-2D show the benchmarking and dose-dependent activation of CXCR4 expression in SLF*-CEMa system. FIG. 2A is a demonstration of the dCas9-based exogenous gene activating technology. FIG. 2B is a demonstration of the SLF*-CEMa technology of recruiting endogenous protein to activate CXCR4 target gene expression. FIG. 2C shows SLF*-CEMa compound activation screening with CXCR4 gRNA or non-targeting (NT) gRNA. All the concentrations of SLF*-CEM molecules were 5 nM in the screening experiment. After 48 hrs of chemical exposure, RNA extraction was conducted and qRT-PCR followed to measure the mRNA expression change of CXCR4. FIG. 2D shows dose curve CXCR4 activation by dCas9 direct fusion and CEM molecules. Lentivirally infected HCT116 cells stably expressing dCas9, MS2-FKBP*x2 constructs were transfected with CXCR4 gRNA or NT gRNA. After 48 hrs of CEM203, CEM207, or CEM87 treatment using concentrations ranging from 0.5 nM to 50 nM, the RNA was extracted and followed by qRT-PCR. CXCR4 mRNA levels were normalized to GAPDH. In FIGS. 2C and 2D, significance was determined by three biological replicates using a two-tailed Student's t-test; P values and fold changes are reported in Table 2. Error bars represent the standard deviation.

FIGS. 3A-3E show the time course, reversibility, and protein level change of SLF*-CEMa targeting CXCR4. FIG. 3A is an illustration of a time course experiment of HCT116 cells. Stable dCas9, MS2-FKBP*x2 expressing cells were transfected with CXCR4 gRNA or NT gRNA. After 24 hrs of SLF*-CEMa treatment, cells were separated into three groups: continuous CEM treatment, washout with media, and washout with 20 μM AP1867 added media. RNA extraction was operated at 12, 24, 48, 72, and 96 hrs time points. CXCR4 mRNA was measured by qRT-PCR. FIG. 3B shows the time course of CXCR4 mRNA expression change with no chemical treatment, 50 nM CEM203 treatment, or 25 nM CEM207 treatment. Both SLF*-CEMa treated groups showed significant CXCR4 gene activation after 12 hrs and reached peak activation at the 24 hrs time point, while no chemical treatment and NT gRNA treated cells showed no activation. FIGS. 3C and 3D show reversibility of SLF*-CEMa activation with CEM203 (FIG. 3C) and CEM207 (FIG. 3D). CXCR4 activation of SLF*-CEMa was reversed in washout and AP1867 added cells. The addition of AP1867 showed lowest activation level among three groups. FIG. 3E shows a dose curve and time course of CXCR4 protein change in HCT116 cells. Stable dCas9 and MS2-FKBP*x2 expressing HCT116 cells were transfected with CXCR4 gRNA or NT gRNA. At 24, 48, and 72 hrs time points after 25 nM CEM207 treatment, cells were collected and the CXCR4 membrane protein APC antibody was used. Flow cytometry was performed to quantify CXCR4 protein expression level. In FIGS. 3B-3E, significance was determined by three biological replicates using a two-tailed Student's t-test; P values and fold changes are reported in Table 2. Error bars represent the standard deviation.

FIGS. 4A-4C show that BRD4 ChIP-seq demonstrates CM207 specificity at the CXCR4 locus. FIG. 4A shows tracks of BRD4 ChIP-seq signal at the CXCR4 locus for four conditions: sgNT, sgCXCR4, sgNT+CEM207, and sgCXCR4+CEM207. The four black bars represent the locations of the four sgCXCR4 target sequences. Signal is read-depth normalized. FIG. 4B is a bar graph of the mean BRD4 signal in the CXCR4 peak across the four conditions. FIG. 4C is a density scatterplot of log₂ fold ChIP-seq signal changes with the addition of either CEM207 (y-axis) or sgCXCR4 (x-axis) in the presence of the other. The 50 most outlying points are marked, including the highlighted CXCR4 region. Significance was determined by three biological replicates using a two-tailed Student's t-test; P values and fold changes are reported in Table 2. Error bars represent the standard deviation.

FIG. 5A shows 12 SLF*-CEMa compound activity screening in TRE3G-GFP reporter system. HCT116 cells were transfected with TRE3G-GFP, dCas9, MS2-FKPB*x2, and TRE3G gRNA or NT gRNA. After 48 hrs of 10 nM SLF*-CEMa treatment, flow cytometry was performed and mean fluorescent value was collected. FIG. 5B shows AP1867 competition assay in HCT116 cell line. TRE3G-GFP, dCas9, MS2-FKBP*2, TRE3G gRNA or NT gRNA were transfected into HCT116 cells. The media was changed to media with 5 nM CEM207 together with 0 nM, 50 nM, 500 nM or 5000 nM of AP1867, respectively. After 48 hrs chemical exposure, flow cytometry was performed and the mean fluorescent value was measured in each group of cells. FIG. 5C is a dose curve of CEMa molecules on reporter gene activation in HCT116 cells (none targeting gRNA groups). HCT116 cells were transfected with dCas9, MS2-FKBP*x2, and NT gRNA. After 48 hrs of different concentrations of CEMa molecule treatments, ranging from 0 nM to 100 nM, flow cytometry was performed and the GFP expression change was detected. FIGS. 5D and 5E are dose curves of CEMa molecules activating the GFP reporter gene in HCT116 cells using WT FKBP and dCas9 as DNA binding module. HCT116 cells were transfected with dCas9, MS2-FKBPx2, TRE3G-GFP constructs, along with TRE3G gRNA or NT gRNA. Cells were treated with different concentrations of CEMa molecules for 48 hrs before flow cytometry was conducted. In FIGS. 5A-5E, significance was determined with three different samples using a two-tailed Student's t-test; fold change and significance are reported in Table 2. Error bars represent the standard deviation.

FIG. 6 is a dose curve of CEMa molecules in HCT116 with WT FKBP system targeting CXCR4. Stable dCas9 and MS2-FKBPx2 expressing HCT116 cells were tranfected with CXCR4 gRNA or NT gRNA. 0 nM, 25 nM, 50 nM, and 200 nM concentrations of CEMa molecules were added to cells for 48 hrs of treatment time. RNA extraction was performed and the CXCR4 expression level was measured by qRT-PCR and normalized to GAPDH. Significance was determined with three different samples using a two-tailed Student's t-test; fold change and significance are reported in Table 2. Error bars represent the standard deviation.

FIG. 7A shows the non-targeting gRNA data of SLF*-CEMa time course and reversibility experiment. HCT116 cells were stably infected with dCas9 and MS2-FKBP*x2 via lentivirus. NT gRNA was transfected and the change of CXCR4 mRNA expression were quantified with RNA extraction and qRT-PCR, normalized to GAPDH. After 24 hrs of SLF*-CEMa exposure, the cells were separated into three groups: 1) continuous SLF*-CEMa treatment; 2) washout with media; and 3) washout with 20 μM AP1867 added. RNA extraction was performed at 12, 24, 48, and 72 hrs time points. FIG. 7B-7D shows CXCR4 protein expression change with 0 nM, 5 nM, 25 nM, and 50 nM of CEM207 treatment at 24, 48, and 72 hrs time points. HCT116 cells were stably expressing dCas9 and MS2-FKBP*x2 constructs and transfected with CXCR4 gRNA or NT gRNA. APC CXCR4 antibody was used in flow cytometry at referred time points. FIG. 7E shows a dose curve of CEM207 in HEK293T cells targeting MYOD1. HEK293T cells were lentivirally infected with dCas9 and MS2-FKBP*x2 constructs and transfected with MYOD1 gRNA or NT gRNA. RNA extraction and qRT-PCR were performed after 24 hrs of CEM207 treatment in 5 nM, 25 nM, and 50 nM concentrations along with no chemical treatment as the control group. MYOD1 mRNA expression level was normalized to GAPDH. FIG. 7F shows activation of CXCR4 gene expression in HEK293T cells. Stable dCas9 and MS2-FKBP*x2 expressing HEK293T cells were transfected with CXCR4 gRNA and NT gRNA. RNA extraction and qRT-PCR were performed after 24 hrs of 25 nM CEM207 or no chemical treatment. CXCR4 mRNA expression level was normalized to GAPDH. FIG. 7G shows activation of IL1RN expression in HCT116 cells. Stable dCas9 and MS2-FKBP*x2 expressing HEK293T cells were transfected with IL1RN gRNA and NT gRNA. RNA extraction and qRT-PCR were performed after 24 hrs of 25 nM CEM207 treatment. IL1RN mRNA expression level was normalized to GAPDH. FIG. 7H shows activation of ASLC1 expression in HCT116 cells. Stable dCas9 and MS2-FKBP*x2 expressing HEK293T cells were transfected with ASLC1 gRNA and NT gRNA. RNA extraction and qRT-PCR were performed after 24 hrs of 25 nM CEM207 treatment. IL1RN mRNA expression level was normalized to GAPDH. In FIGS. 7A-7F, significance was determined with three different samples using a two-tailed Student's t-test; fold changes and significance are reported in Table 2. Error bars represent the standard deviation.

FIGS. 8A-8B show alamarBlue cell viability assays. HEK293T or HCT116 cells were plated in 96-well plates 24 hrs before chemical treatment of 0.001, 0.01, 0.1, 1, 10, 50, and 100 μM CEM207 was added. Another group of cells were treated with all concentrations of CEM207 mentioned above along with 200 nM CEM87 in all samples. After 4 days of chemical exposure, cell growth was measured by fluorescence. FIG. 8C shows ChlP-qPCR. HCT116 cells were stably infected with dCas9 and MS2-FKBP*x2 constructs and transfected with CXCR4 gRNA. After 24 hrs of 25 nM CEM207 treatment, ChIP-qPCR was performed at different base pair distances from the transcription start site of CXCR4 gene. In FIGS. 8A-8C, significance was determined with three different samples using a two-tailed Student's t-test; fold change and significance are reported in Table 2. Error bars represent the standard deviation.

FIGS. 9A-9B show a flow cytometry gating strategy in experiments. All live cells were gated by forward scatter area (FSC-A) to side scatter area (SSC-A) and all single cells were gated by FSC-A and SSC-A to determine the GFP signal collected in experiments.

FIG. 10 is a schematic of an exemplary approach for rapid screening of CEM technology using dual luciferase assays.

FIGS. 11A-11B show preliminary regulation of AAV with first generation CEMs. An exemplary CEM molecule binding FKBP and BRD4 (FIG. 11A) with an expression cassette comprising a 3384 base pair expression cassette including a JET promoter, a ZFHD1-FKBP chimeric anchor protein and firefly luciferase (FIG. 11B).

FIG. 12 shows results of preliminary regulation of AAV with an exemplary first generation CEM at 50 nM, 200 nM, 500 nM and 1000 nM in HEK293T Renilla, left, and HCT116 Renilla (right) as measured by fold change in luminescence compared to no treatment (0 nM CEM) using the expression cassette of FIG. 11B in HEK293T Renilla.

FIG. 13 shows results of preliminary regulation of AAV with an exemplary first generation CEM with increasing number of viral genomes/cell (vg/cell) at concentrations of 200 nM and 500 nM CEM87 in HEK293T Renilla.

FIGS. 14A-14C show an expression cassette for use with SLF* CEM (FIG. 14A) and normalized luminescence (FIG. 14B) and fold change from 0 nM (FIG. 14C) of exemplary CEM207 at increasing number of vg/cell indicates SLF* CEM activation of rAAV2 is compatible with viral titer escalation.

FIGS. 15A-15B show normalized luminescence (FIG. 15A) and fold change (FIG. 15B) results of a study of systems with varied length promoters with an exemplary SLF* CEM targeting system showing a variety of minimal promoters are well-tolerated using the expression cassettes depicted.

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 4^{th} Ed. (Cold Spring Harbor, NY, 2012); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### General Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together.

The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “tropism” as used herein refers to preferential but not necessarily exclusive entry of the vector (e.g., virus vector) into certain cell or tissue type(s) and/or preferential but not necessarily exclusive interaction with the cell surface that facilitates entry into certain cell or tissue types, optionally and preferably followed by expression (e.g., transcription and, optionally, translation) of sequences carried by the vector contents (e.g., viral genome) in the cell, e.g., for a recombinant virus, expression of the heterologous nucleotide sequence(s).

The term “tropism profile” refers to the pattern of transduction of one or more target cells, tissues and/or organs. Representative examples of chimeric AAV capsids have a tropism profile characterized by efficient transduction of cells of the central nervous system (CNS) with only low transduction of peripheral organs (see e.g., U.S. Pat. No. 9,636,370 Mccown et al., and US patent publication 2017/0360960 Gray et al.). Vectors (e.g., virus vectors, e.g., AAV capsids) expressing specific tropism profiles may be referred to as “tropic” for their tropism profile, e.g., neuro-tropic, liver-tropic, etc.

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide,” “nucleic acid,” or “nucleotide sequence” may be of RNA, DNA or DNA-RNA hybrid sequences (including both naturally occurring and non-naturally occurring nucleotides) but is preferably either a single or double stranded DNA sequence.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. The region in a nucleic acid sequence or polynucleotide in which one or more regulatory elements are found may be referred to as a “regulatory region.”

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “codon-optimized,” as used herein, refers to a gene coding sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, the term “modified,” as applied to a polynucleotide or polypeptide sequence, refers to a sequence that differs from a wildtype sequence due to one or more deletions, additions, substitutions, or any combination thereof.

As used herein, by “isolate” (or grammatical equivalents) a virus vector, it is meant that the virus vector is at least partially separated from at least some of the other components in the starting material.

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease and encompass any type of prophylactic treatment to reduce the incidence of the condition or delay the onset of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

A “heterologous nucleotide sequence” or “heterologous nucleic acid,” with respect to a virus or other vector, is a sequence or nucleic acid, respectively, that is not naturally occurring in the virus or other vector. Generally, the heterologous nucleic acid or nucleotide sequence comprises an open reading frame that encodes a polypeptide and/or a nontranslated RNA.

A “vector” refers to a compound used as a vehicle to carry foreign genetic material into another cell, where it can be replicated and/or expressed. A vector containing foreign or heterologous nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to as transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the term “viral vector” and “delivery vector” (and similar terms) in a specific embodiment generally refers to a virus particle that functions as a nucleic acid delivery vehicle, and which comprises the viral nucleic acid (i.e., the vector genome) packaged within the virion. Viral vectors according to the present invention may include chimeric AAV capsids according to the invention and can package an AAV or rAAV genome or any other nucleic acid including viral nucleic acids. Alternatively, in some contexts, the terms “viral vector” and “delivery vector” (and similar terms) may be used to refer to the vector genome (e.g., vDNA) in the absence of the virion and/or to a viral capsid that acts as a transporter to deliver molecules tethered to the capsid or packaged within the capsid.

The term “template” or “substrate” is used herein to refer to a polynucleotide sequence that may be replicated to produce the viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, the term “amino acid” encompasses any naturally occurring amino acids, modified forms thereof, and synthetic amino acids, including non-naturally occurring amino acids. Alternatively, the amino acid can be a modified amino acid residue or can be an amino acid that is modified by post-translation modification (e.g., acetylation, amidation, formylation, hydroxylation, methylation, phosphorylation or sulfatation). The non-naturally occurring amino acid can be an “unnatural” amino acid as described by Wang et al., (2006) Annu. Rev. Biophys. Biomol. Struct. 35:225-49.

A “functional fragment” of a polypeptide or protein, as used herein, means a portion of a larger polypeptide that substantially retains at least one biological activity normally associated with that polypeptide (e.g., wild-type protein or fragment thereof). In particular embodiments, the “functional” polypeptide or “functional fragment” substantially retains all of the activities possessed by the unmodified polypeptide (e.g., wild-type protein or fragment thereof). By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as binding activity can be measured using assays that are well known in the art and as described herein.

The term “fragment,” as applied to a peptide, will be understood to mean an amino acid sequence of reduced length relative to a reference peptide (e.g., wild-type protein) or amino acid sequence and comprising, consisting essentially of, and/or consisting of an amino acid sequence of contiguous amino acids identical to the reference peptide or amino acid sequence. Such a peptide fragment according to the invention may be, where appropriate, included in a larger polypeptide of which it is a constituent. In some embodiments, such fragments can comprise, consist essentially of, and/or consist of peptides having a length of at least about 5, 10, 15, 20, 25, 30, 35, 46, 50, 55, 60, 65, 70, 75, 80, 85, 90, 95 or 100 or more consecutive amino acids of a peptide or amino acid sequence according to the invention.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof refers to bringing two or more substances in sufficiently close proximity to each other for one to exert a biological effect on the other.

As used herein, the term “derivative” is used to refer to a polypeptide which differs from a naturally occurring protein or a functional fragment by minor modifications to the naturally occurring polypeptide, but which substantially retains the biological activity of the naturally occurring protein. Minor modifications include, without limitation, changes in one or a few amino acid side chains, changes to one or a few amino acids (including deletions, insertions, and/or substitutions) (e.g., less than about 20, 15, 10, 9, 8, 7, 6, 5, 4, 3, or 2 changes), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol.

The term “substantially retains,” as used herein, refers to a fragment, derivative, or other variant of a polypeptide that retains at least about 50% of the activity of the naturally occurring polypeptide (e.g., binding to recognition sequence), e.g., about 60%, 70%, 80%, 90% or more.

### Compositions for Modulating Gene Expression

Compositions are described herein for modulating gene expression, including transgene expression. The compositions include bifunctional chemical epigenetic modifiers (CEMs). The bifunctional CEMs comprise a molecule for binding FK506 binding protein (FKBP), Reference Sequence NM_00801.3. In an embodiment, the FKBP is an F36V mutant (FKBP^{F36V}) (MGVQVETISPGDGRTFPKRGQTCVVHYTGMLEDGKKVDSSRDRNKPFKFMLGKQEVI RGWEEGVAQMSVGQRAKLTISPDYAYGATGHPGIIPPHATLVFDVELLKLE (SEQ ID NO: 1)) (See, Clackson et al., Proc. Natl.: Acad. Sci. USA. 1998; 95:10437-10442). Thus, the CEMs of the present invention can be used with a fusion protein comprising FKBP^{F36V}. In an embodiment, the CEM can comprise a FKBP^{F36V} binding molecule, a linker, and a chromatin regulatory protein ligand. The CEMs can be utilized with a fusion protein and additional elements to allow for endogenous gene or transgene modulation, as described further herein. The CEM allows for reversible and dose-dependent control of gene modulation.

In certain embodiments, the CEM binds to a chromatin regulatory protein, e.g., transcriptional activator protein or complex or transcriptional repressor protein or complex, that when recruited to the target gene or the transgene delivery vector modulates expression of the gene or transgene. Examples of chromatin regulatory proteins include, without limitation, BRD4, HDAC, or CBP/p300. The CEMs of the present invention are capable of binding to a chromatin regulatory protein that can be activating or repressing, and as shown herein, can control recombinant AAV transgene expression in transduced cells.

For example, the CEM can comprise a BET binding molecule that is capable of binding a BET protein, e.g., BRD4, or, the CEM can comprise a suberoylanilide hydroxamic acid (SAHA) molecule, or derivative thereof, that is capable of binding HDAC.

An exemplary CEM that binds FK506 binding protein with F36V mutation and a chromatin regulatory protein is according to Formula I:

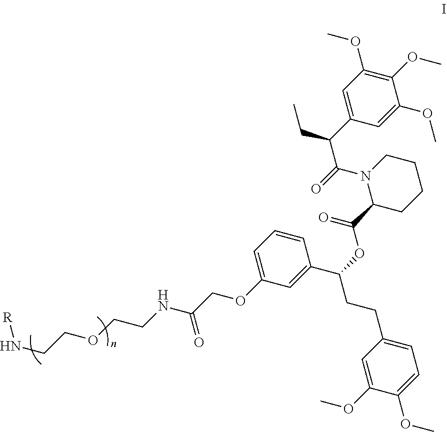

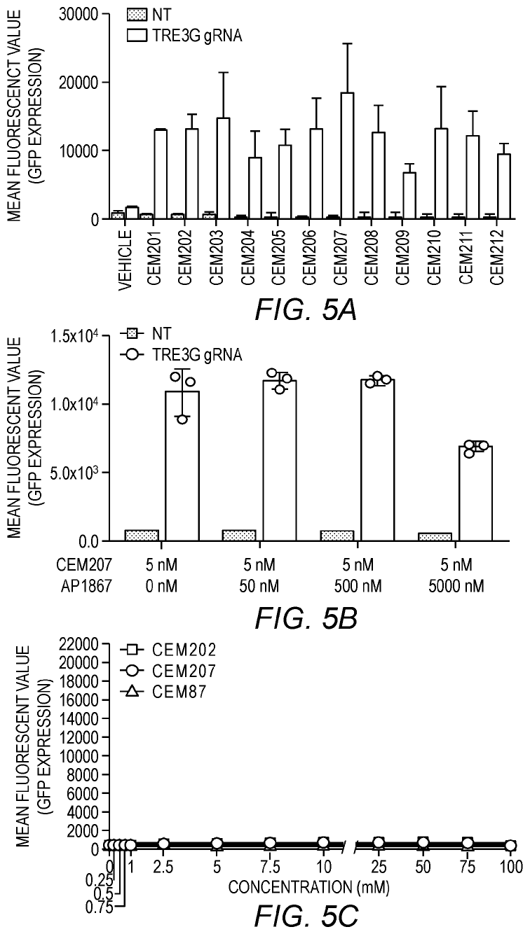

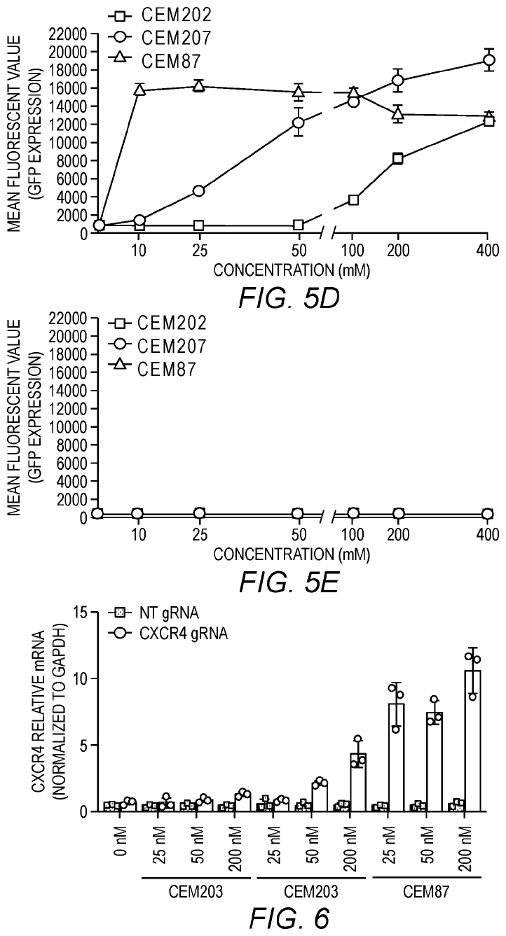

or a pharmaceutically acceptable salt thereof, wherein: n is 1-10; and R is a chromatin regulatory protein ligand. In an aspect, the chromatin regulatory protein ligand is a ligand specific for BET, for example, (+)-JQ1-, I-BET762, N-[3-(2-oxo-pyrrolidinyl)phenyl]-benzenesulfonamide derivatives, e.g., Enamine ID Nos. Z115668302, Z115668110, Z115668200, Z115668236, Z115668228, and Z115668152 (Allen et al., ACS Omega. 2017 Aug. 31; 2 (8): 4760-4771), apabetalone, and 8-Methyl-pyrrolo[1,2-a]pyrazin-1 (2H)-one derivatives such as compound 38 (Li et al., J. Med. Chem. 2020, 63, 8, 3956-3975). In an aspect, the chromatin regulatory protein ligand is a ligand specific for HDAC, e.g., tubacin, trichostatin, vorinostat (SAHA), belinostat, Panobinostat, mocetinostat, or entinostat, See, Shukla, et al., Front. Pharmacol. 11:537 (2020)) at FIG. 5, incorporated herein by reference in its entirety. In some embodiments, n is between 1 and 10, e.g., 1, 2, 3, 4, 5, 6, 7, 8, 9, 10, between 1 and 7, between 2 and 6, or between about 2 and 5.

An exemplary CEM that binds FK506 binding protein with F36V mutation and BRD4 is according to Formula Ia:

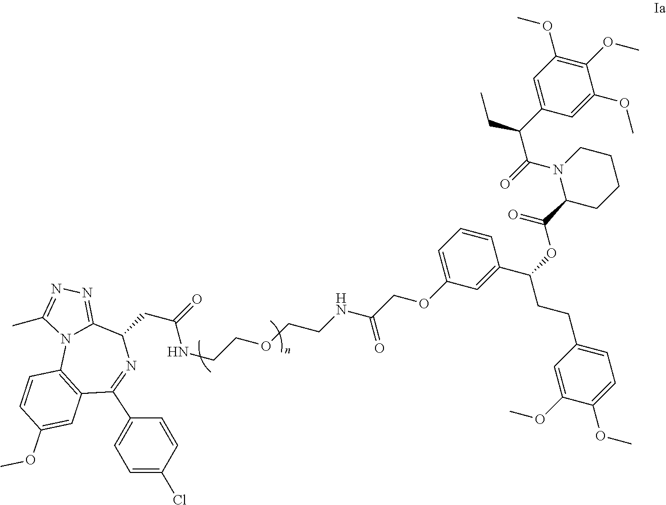

or a pharmaceutically acceptable salt thereof.

An exemplary CEM that binds FK506 binding protein with F36V mutation and BRD4 is according to Formula Ib:

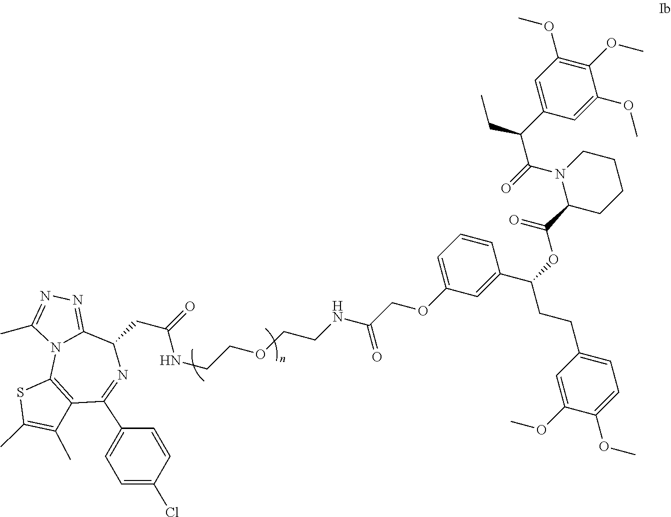

or a pharmaceutically acceptable salt thereof.

An exemplary CEM that binds FK506 binding protein with F36V mutation and HDAC is according to Formula Ic:

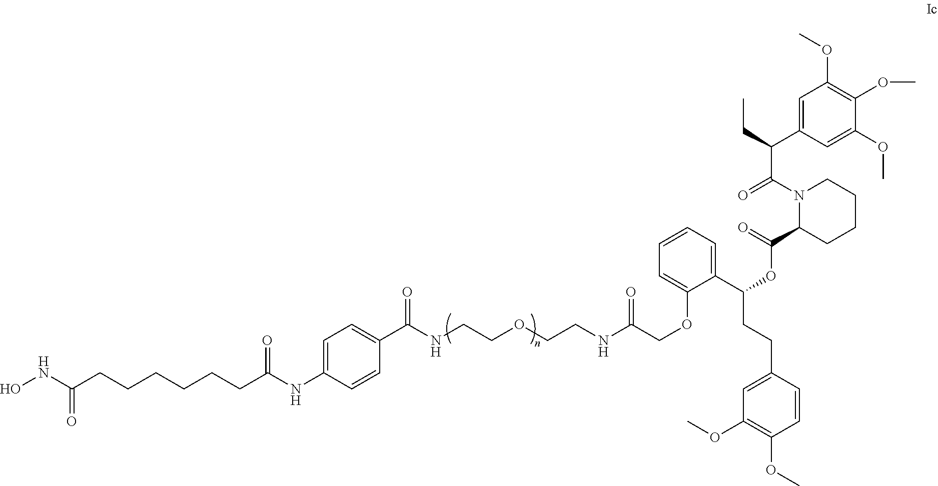

or a pharmaceutically acceptable salt thereof.

Compositions of the present invention can comprise the CEMs as described herein and a FK506-binding protein (FKBP) with a F36V mutation. In an aspect, the FKBP is provided in a fusion protein, and can comprise one or more additional components allowing for sequence specific binding of a target gene, thereby allowing the CEM to bind the fusion protein and an endogenous chromatin regulatory protein, allowing for modulation of genes, as described further herein. In some embodiments, the FKBP^{F36V} mutant binds a bumped CEM ligand (see, e.g., Lu, et al., ACS Synth. Biol. 11:1397 (2022), incorporated by reference herein in its entirety). In some embodiments, the FKBP^{F36V} mutation is provided as a fusion protein. The portion of the fusion protein that is a domain that binds a CEM may be any polypeptide that specifically recognizes and binds to a portion of the CEM that is a ligand for the domain, e.g., the domain that binds a CEM is FK506 binding protein comprising an F36V mutation, FKBP^{F36V}.

In some embodiments, the fusion protein further comprises an RNA binding domain, e.g., a guide RNA (gRNA) binding polypeptide, which may be an MS2 sequence. In some embodiments, the fusion protein comprising a guide RNA binding polypeptide is encoded by a polynucleotide which may be comprised in an expression cassette. Fusion proteins can be utilized with CRISPR-cas systems to modulate target gene expression, as described further herein.

In some embodiments, the fusion protein further comprises one or more DNA binding domains, for example, one or more zinc fingers. In some embodiments, the fusion protein comprising one or more DNA binding domains is encoded by a polynucleotide which may be comprised in an expression cassette. In some embodiments, the fusion proteins are utilized with transgenes and provided in delivery vectors to modulate transgene expression, as described further herein.

### CRISPR-Cas Compositions

In some embodiments, the fusion protein is designed to work with a CRISPR-Cas system. Compositions of the present invention can comprise the fusion protein and a CRISPR-Cas system, e.g., a CRISPR-Cas protein and a guide RNA (gRNA). In some instances, the fusion protein comprises an FK506-binding protein (FKBP) with a F36V mutation and an RNA binding domain, e.g., a guide RNA (gRNA) binding polypeptide. In some embodiments, the RNA binding polypeptide of the fusion protein is recognized by (capable of binding) an MS2, PP7, GA or Qβ hairpin loop that is comprised on the gRNA. In an embodiment, the RNA binding domain can be an MS2 coat protein that binds the MS2 hairpin loop engineered on the gRNA. In one embodiment, the fusion protein comprises an MS2-FKBP^{F36V}x2. Exemplary systems used with Cas9, and which can be similarly used with other Cas proteins, are described, for example, in Konermann, S. et al. Genome-scale transcriptional activation by an engineered CRISPR-Cas9 complex. Nature 517, 583-588 (2015), incorporated herein by reference in its entirety.

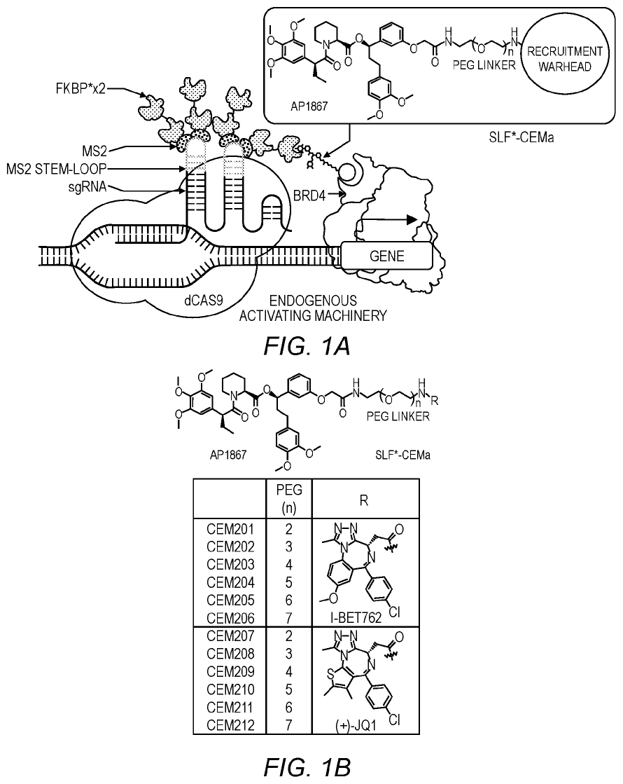

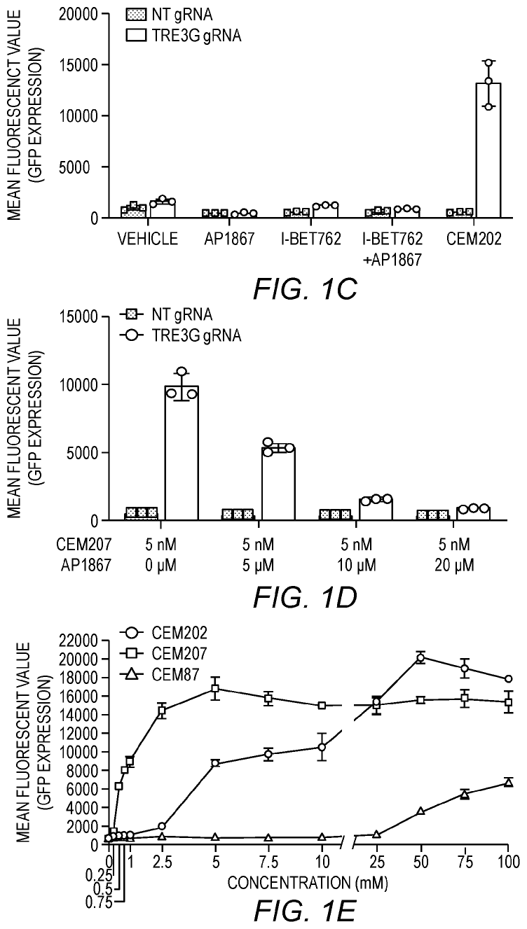

CRISPR-Cas proteins are known in the art. Gene editing systems can also be utilized, which may comprise a CRISPR system, a zinc finger nuclease system, or a TALE system. A CRISPR-Cas system can comprise a Class 1 or Class 2 CRISPR-Cas system, which may comprise a guide sequence engineered to specifically bind a polynucleotide of interest. The CRISPR-Cas system that can be used to modify expression of a polynucleotide of the present invention described herein can be a Class 1 CRISPR-Cas system. Class 1 CRISPR-Cas systems are divided into types I, II, and IV. Makarova et al. 2020. Nat. Rev. 18:67-83., particularly as described in FIG. 1. Type I CRISPR-Cas systems include Types I-A, I-B, I-C, I-D, I-E, I-F1, I-F2, I-F3, and IG; Type III CRISPR-Cas systems can be Types III-A, III-B, III-C, III-D, III-E, and III-F; which can contain a Cas10 that can include an RNA recognition motif called Palm and a cyclase domain that can cleave polynucleotides; Type IV CRISPR-Cas systems include Types IV-A, IV-B, and IV-C. Class 2 systems comprise a single, large, multi-domain effector protein and can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Class 2, Type II systems include II-A, II-B, II-C1, and II-C2; Type V systems include V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type IV systems include VI-A, VI-B1, VI-B2, VI-C, and VI-D. Design of guides for targeting a nucleic acid for modification is known in the art, see, e.g., IDTdna.com and Synthego.com for guidance on custom guide RNAs. Reduction of off-target effects can be tailored using programs such as GUIDE-seq. See, e.g., Malinin, et al., Nature Protocols, 16, 5592-5615 (2012). In one embodiment, the CRISPR-Cas protein is a DNA binding protein. In an embodiment, the Cas protein is a Cas9 or Cas12a protein.

In one embodiment, the protein with a DNA binding domain is a deactivated protein (dCas9) which comprises a D10A mutation in the RuvC domain and an H840A mutation in the HNH nuclease domain of SpCas9 (Ref. Seq. WP_038431314.1), or corresponding amino acids of the RuvC domain and HNH domain of other Cas9 proteins. See, e.g., Anderson et al., Molecular Systems Biology (2021) 17: e10512; Jiang F, Doudna J A CRISPR-Cas9 structures and mechanisms. Annu Rev Biophys 46:505-529 (2017).

The gRNA can be engineered to comprise a stem loop, e.g., hairpin motif, that can bind the RNA binding domain of the fusion protein. For example, the gRNA may be engineered to comprise an MS2 stem-loop (hairpin motif) recognized by the MS2 polypeptide of the fusion protein. The gRNA is engineered to comprise a gene targeting polynucleotide sequence and a protein binding polynucleotide sequence. The gene targeting polynucleotide sequence can be selected according to the desired target in the cell. The gRNA can be further engineered according to the protein with a DNA binding domain and recognition site for the gRNA utilized, e.g., CRIPSR_Cas protein. Thus, in some embodiments, the protein binding polynucleotide sequence of the gRNA forms a MS2 stem-loop motif recognized by the fusion proteins described herein. The gRNA is capable of forming a complex with a CRISPR-Cas protein, binding the target sequence in the cell, thereby allowing for sequence specific binding of the target gene, which is also bound to the fusion protein via the binding of the gRNA binding polypeptide to the gRNA. The CEM binds the FKBP^{F36V} polypeptide of the fusion protein and binds a chromatin regulatory protein via its warhead, thereby recruiting the chromatin regulatory protein, e.g., endogenous transcriptional regulatory protein or complex machinery of the cell via the CEM molecules described herein, to the target gene. Modulation of the target gene by the endogenous transcriptional regulatory protein or complex can then be affected.

CEMs useful in the methods of the invention may be any bifunctional small molecule (e.g., less than 1500 Da) that is capable of binding and/or binds the fusion protein of the invention and a chromatin regulatory protein. CEMs with chromatin regulatory protein ligands that can be modified for use in the present invention can include, without limitation, those described in International Patent Publication Nos. WO 2019/028426 and WO 2022/236010, incorporated herein by reference in their entirety. Fusion proteins can be designed to include a polypeptide capable of binding known CEMs with a guide RNA binding polypeptide, and further with the CRISPR-Cas systems as described herein.

### Delivery Vectors for Transgene Modulation

Fusion proteins of the present invention can be used with delivery vectors, for example, a recombinant AAV, that comprises a DNA binding domain recognition polynucleotide. The fusion protein can comprise the FKBP^{F36V} polypeptide and a DNA binding domain specific for the DNA binding domain recognition polynucleotide in the delivery vector. In embodiments, the fusion protein is encoded by a polynucleotide that can be comprised in an expression cassette. In some embodiments the expression cassette can further comprise a transgene that can be transduced into a vector, for example AAV.

In some embodiments, the nucleic acid binding domain of the fusion protein is a DNA binding domain. In some embodiments, the DNA binding domain can comprise one or more zinc finger DNA binding domain, a helix-loop-helix DNA binding domain, a bZIP DNA binding domain, an HMG-box DNA binding domain, a transcription activator-like effector DNA binding domain, a transcription factor DNA binding domain, or a restriction endonuclease DNA binding domain. In particular embodiments, the DNA binding domain may be, without limitation, a domain from GALA, LexA, GCN4, THY1, SYN1, NSE/RU5′, AGRP, CALB2, CAMK2A, CCK, CHAT, DLX6A, EMX1, Cas9, Cas3, Cas4, Cas5, Cas5e (or CasD), Cash, Cas6e, Cas6f, Cas7, Cas8a1, Cas8a2, Cas8b, Cas8c, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu196, or TALES. In certain embodiments, the protein comprises one or more zinc finger proteins or TALENS that bind sequence specific DNA.

The compositions can comprise a transgene which may encode any product for which delivery and expression is desired, as discussed further below. In some embodiments, the transgene encodes a protein. In other embodiments, the transgene encodes a functional nucleic acid, e.g., an antisense nucleic acid or an inhibitory RNA.

The delivery vector may comprise a nucleic acid binding domain recognition sequence and a polynucleotide encoding the transgene expression cassette. The nucleic acid binding domain recognition sequence serves as a binding site for the fusion protein comprising a DNA binding domain that binds to the recognition sequence fused to a domain that binds a CEM. In some embodiments, the polynucleotide further comprises a sequence encoding the fusion protein. In these embodiments, the transgene and the sequence encoding the fusion protein may be operably linked to separate promoters (which may be the same promoter or different promoters) or may be operably linked to a single promoter, which may optionally be a bidirectional promoter. In other embodiments, the fusion protein may be expressed from the sequence encoding the fusion protein due to the inherent promoter activity of AAV ITRs. In an embodiment, the promoter can be between about 25 base pairs to about 600 base pairs in length, for example, 25, 35, 45, 55, 65, 75, 85, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 base pairs. Exemplary promoters for the expression cassettes can include Jet, ybTATA, miniCMV, Het, Ef1a core, and hPGK.

The nucleic acid binding domain recognition sequence may be any nucleotide sequence that is specifically recognized and bound by a nucleic acid binding protein (DNA binding protein) such that the presence of the nucleic acid binding domain recognition sequence in the transgene delivery vector recruits a fusion protein comprising the nucleic acid binding protein. The nucleic acid binding domain recognition sequence may comprise two or more sequences (e.g., 2, 3, 4, 5, 6, or sequences) that are binding sites for the nucleic acid binding protein such that two or more nucleic acid binding proteins bind to a delivery vector, e.g., transgene delivery vector. For example, the nucleic acid binding domain recognition sequence may comprise 6 binding sites for zinc finger proteins so that a fusion protein comprising 6 zinc finger proteins may bind the transgene delivery vector in a sequence specific manner. In one embodiment, the DNA binding protein is a zinc finger binding domain and the nucleic acid binding domain recognition sequence is a zinc finger binding domain recognition sequence as described, for example in International Patent Publication No. WO 2022/236010, for example, at FIG. 5 and [0215]-[0217]. In other embodiments, the multiple binding sites may recruit multiple fusion proteins to amplify the expression modulation to levels greater than can be achieved by a single fusion protein.

The nucleic acid binding domain recognition sequence may be recognized by a protein (e.g., zinc finger proteins) or a nucleic acid (e.g., RNA guided Cas proteins).

### Delivery Vectors

The delivery vector may be any type of vector known to be useful for delivering a polynucleotide to a cell. In some embodiments, the delivery vector is a viral vector, e.g., a viral genome. Examples of viral vectors include, without limitation, an adeno-associated virus, retrovirus, lentivirus, poxvirus, alphavirus, baculovirus, vaccinia virus, herpes virus, Epstein-Barr virus, or adenovirus vector. As used herein, the term “transgene delivery vector” refers to a delivery vector capable of delivering a transgene to a cell or to a subject and expressing the transgene in the cell or subject; the transgene delivery vector may utilize the delivery vectors described herein.

In some embodiments, the delivery vector is a non-viral vector. Examples of non-viral vectors include, without limitation, a plasmid, liposome, electrically charged lipid, nucleic acid-protein complex, or biopolymer.

Another aspect of the invention relates to a cell comprising the transgene delivery vector of the invention. The cell may be in vitro or in vivo.

A further aspect of the invention relates to a composition comprising the delivery vector or the cell of the invention, e.g., a pharmaceutical composition comprising the transgene delivery vector or the cell of the invention and a pharmaceutically acceptable carrier. In some embodiments, the pharmaceutical composition further comprises the fusion protein and/or the CEM of the invention.

In some embodiments of the invention, the delivery vector is a parvovirus vector. The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al., VIROLOGY, volume 2, chapter 69 (4th ed., Lippincott-Raven Publishers).

In some embodiments of the invention, the transgene delivery vector is a parvovirus within the genus Dependovirus. The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al., VIROLOGY, volume 2, chapter 69 (4th ed., Lippincott-Raven Publishers); and Table 1. A number of additional AAV serotypes and clades have been identified (see, e.g., Gao et al., (2004) J. Virol. 78:6381-6388 and Table 1), which are also encompassed by the term “AAV.”

As discussed above, the parvovirus particles and genomes of the present invention can be from, but are not limited to, AAV. The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching parvovirus and AAV nucleic acid and amino acid sequences. See also, e.g., Bantel-Schaal et al., (1999) J. Virol. 73:939; Chiorini et al., (1997) J. Virol. 71:6823; Chiorini et al., (1999) J. Virol. 73:1309; Gao et al., (2002) Proc. Nat. Acad. Sci. USA 99:11854; Moris et al., (2004) Virol. 33-: 375-383; Mori et al., (2004) Virol. 330:375; Muramatsu et al., (1996) Virol. 221:208; Ruffing et al., (1994) J. Gen. Virol. 75:3385; Rutledge et al., (1998) J. Virol. 72:309; Schmidt et al., (2008) J. Virol. 82:8911; Shade et al., (1986) J. Virol. 58:921; Srivastava et al., (1983) J. Virol. 45:555; Xiao et al., (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein it its entirety).

The term “AAV viral vectors” includes “chimeric” AAV nucleic acid capsid coding sequence or AAV capsid protein is one that combines portions of two or more capsid sequences. A “chimeric” AAV virion or particle comprises a chimeric AAV capsid protein.

TABLE 1

| AAV | GenBank |
|---|---|
| Serotypes/Isolates | Accession Number |
| Clonal Isolates | |
| Avian AAV | AY186198, AY629583, |
| ATCC VR-865 | NC_004828 |
| Avian AAV | NC_006263, AY629583 |
| strain DA-1 | |
| Bovine AAV | NC_005889, AY388617 |
| AAV4 | NC_001829 |
| AAV5 | AY18065, AF085716 |
| Rh34 | AY243001 |
| Rh33 | AY243002 |
| Rh32 | AY243003 |
| AAV10 | AY631965 |
| AAV11 | AY631966 |
| AAV12 | DQ813647 |
| AAV13 | EU285562 |
| Clade A | |
| AAV1 | NC_002077, AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu19 | AY530584 |
| Hu20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| AAV 3 | NC_001729 |
| AAV 3B | NC_001863 |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| AAV9 (Hu14) | AY530579 |
| Hu31 | AY530596 |
| Hu32 | AY530597 |

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged. The virus vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al., (2000) Mol. Therapy 2:619.

The AAV viral vectors of the invention may include a recombinant AAV vector genome. A “recombinant AAV vector genome” or “rAAV genome” is an AAV genome (i.e., vDNA) that comprises at least one inverted terminal repeat (e.g., one, two or three inverted terminal repeats) and one or more heterologous nucleotide sequences. rAAV vectors generally retain the 145 base terminal repeat(s) (TR(s)) in cis to generate virus; however, modified AAV TRs and non-AAV TRs including partially or completely synthetic sequences can also serve this purpose. All other viral sequences are dispensable and may be supplied in trans (Muzyczka, (1992) Curr. Topics Microbiol. Immunol. 158:97). The rAAV vector optionally comprises two TRs (e.g., AAV TRs), which generally will be at the 5′ and 3′ ends of the heterologous nucleotide sequence(s) but need not be contiguous thereto. The TRs can be the same or different from each other. The vector genome can also contain a single ITR at its 3′ or 5′ end. The terms “rAAV particle” and “rAAV virion” are used interchangeably here. A “rAAV particle” or “rAAV virion” comprises a rAAV vector genome packaged within an AAV capsid.

The term “terminal repeat” or “TR” includes any viral terminal repeat or synthetic sequence that forms a hairpin structure and functions as an inverted terminal repeat (ITR) (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or the SV40 hairpin that serves as the origin of SV40 replication can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al., VIROLOGY, volume 2, chapters 69 & 70 (4th ed., Lippincott-Raven Publishers).

An “AAV terminal repeat” or “AAV TR” may be from any AAV, including but not limited to serotypes 1, 2, 3, 4, 5, 6, 7, 8, 9, 10, 11, 12, or 13 or any other AAV now known or later discovered (see, e.g., Table 1). An AAV terminal repeat need not have the native terminal repeat sequence (e.g., a native AAV TR sequence may be altered by insertion, deletion, truncation and/or missense mutations), as long as the terminal repeat mediates one or more of the desired functions, e.g., replication, virus packaging, integration, and/or provirus rescue, and the like.

Further, the viral capsid or genomic elements can contain other modifications, including insertions, deletions and/or substitutions.

As used herein, parvovirus or AAV “Rep coding sequences” indicate the nucleic acid sequences that encode the parvoviral or AAV non-structural proteins that mediate viral replication and the production of new virus particles. The parvovirus and AAV replication genes and proteins have been described in, e.g., FIELDS et al., VIROLOGY, volume 2, chapters 69 & 70 (4th ed., Lippincott-Raven Publishers).

The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wildtype or synthetic. A wildtype large Rep protein may be from any parvovirus or AAV, including but not limited to serotypes 1, 2, 3a, 3b, 4, 5, 6, 7, 8, 9, 10, 11, or 13, or any other AAV now known or later discovered (see, e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another. Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al., (2002) Human Gene Therapy 13:1935).

As used herein, the parvovirus or AAV “cap coding sequences” encode the structural proteins that form a functional parvovirus or AAV capsid (i.e., can package DNA and infect target cells). Typically, the cap coding sequences will encode all of the parvovirus or AAV capsid subunits, but less than all of the capsid subunits may be encoded as long as a functional capsid is produced. Typically, but not necessarily, the cap coding sequences will be present on a single nucleic acid molecule.

The capsid structure of autonomous parvoviruses and AAV are described in more detail in BERNARD N. FIELDS et al., VIROLOGY, volume 2, chapters 69 & 70 (4th ed., Lippincott-Raven Publishers).

In some embodiments, the transgene delivery vector encodes a protein or nucleic acid. In some embodiments, the protein is an enzyme, a regulatory protein, or a structural protein, e.g., one that can substitute for a missing or defective protein in a subject. In some embodiments, the nucleic acid is a functional nucleic acid, e.g., an antisense nucleic acid or an inhibitory RNA.

Any nucleic acid sequence(s) of interest may be delivered in the transgene delivery vectors of the present invention. Nucleic acids of interest include nucleic acids encoding polypeptides, including therapeutic (e.g., for medical or veterinary uses), immunogenic (e.g., for vaccines), or diagnostic polypeptides.

Therapeutic polypeptides include, but are not limited to, cystic fibrosis transmembrane regulator protein (CFTR), dystrophin (including mini- and micro-dystrophins (see, e.g., Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, a1-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), lysosomal acid α-glucosidase, α-galactosidase A, receptors (e.g., the tumor necrosis growth factor α soluble receptor), S100A1, parvalbumin, adenylyl cyclase type 6, a molecule that effects G-protein coupled receptor kinase type 2 knockdown such as a truncated constitutively active bARKct, anti-inflammatory factors such as IRAP, anti-myostatin proteins, aspartoacylase, and monoclonal antibodies (including single chain monoclonal antibodies; an exemplary Mab is the Herceptin® Mab). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. Parvovirus vectors can also be used to deliver monoclonal antibodies and antibody fragments, for example, an antibody or antibody fragment directed against myostatin (see, e.g., Fang et al., Nature Biotechnol. 23:584-590 (2005)).

Nucleic acid sequences encoding polypeptides include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

Alternatively, in particular embodiments of this invention, the nucleic acid may encode a functional nucleic acid, i.e., nucleic acid that functions without getting translated into a protein, e.g., an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), RNAi to a sarcoglycan [e.g., α, β, γ], RNAi against myostatin, myostatin propeptide, follistatin, or activin type II soluble receptor, RNAi against anti-inflammatory polypeptides such as the Ikappa B dominant mutant, and RNAi directed against pathogenic organisms and viruses (e.g., hepatitis B virus, human immunodeficiency virus, CMV, herpes simplex virus, human papilloma virus, etc.).

Alternatively, in particular embodiments of this invention, the nucleic acid may encode protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, enos, inos, or bone morphogenic proteins (including BNP 2, 7, etc., RANKL and/or VEGF).

The transgene delivery vectors may also comprise a nucleic acid that shares homology with and recombines with a locus on a host chromosome. This approach can be utilized, for example, to correct a genetic defect in the host cell.

The present invention also provides transgene delivery vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. Nos. 5,882,652, 5,863,541 to Samulski et al.). The antigen may be presented in the parvovirus capsid. Alternatively, the antigen may be expressed from a nucleic acid introduced into a recombinant vector genome. Any immunogen of interest as described herein and/or as is known in the art can be provided by the nucleic acid delivery vectors.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia L1 or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

It will be understood by those skilled in the art that the nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements or cell stress-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the nucleic acid sequence(s) is transcribed and then translated in the target cells, specific initiation signals are generally included for efficient translation of inserted protein coding sequences. These exogenous translational control sequences, which may include the initiation codon (e.g., ATG) and adjacent sequences, can be of a variety of origins, both natural and synthetic.

The cell(s) into which the transgene delivery vector is introduced can be of any type, including but not limited to neural cells (including cells of the peripheral and central nervous systems, in particular, brain cells such as neurons and oligodendrocytes), lung cells, cells of the eye (including retinal cells, retinal pigment epithelium, and corneal cells), blood vessel cells (e.g., endothelial cells, intimal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, kidney cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above. Furthermore, the cells may be dividing or non-dividing.

Embodiments of the invention may be performed in vitro or in vivo. One aspect of the present invention is a method of expressing a transgene in a cell in vitro, e.g., for research purposes or as part of an ex vivo method. The transgene delivery vector may be introduced into the cells at the appropriate amount, e.g., multiplicity of infection for a viral vector, according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, more preferably at least about 10⁵ infectious units are introduced to the cell.

In particular embodiments, the cells have been removed from a subject, the transgene delivery vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the transgene delivery vectors can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the transgene delivery vector are administered to the subject in a treatment effective or prevention effective amount in combination with a pharmaceutical carrier.

The transgene delivery vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The transgene delivery vectors can also be used to produce a polypeptide of interest or functional RNA in a subject (e.g., using the subject as a bioreactor to produce the polypeptide or to observe the effects of the functional nucleic acid on the subject, for example, in connection with screening methods). The transgene delivery vectors may also be employed to provide a functional nucleic acid to a cell in vitro or in vivo. Expression of the functional nucleic acid in the cell, for example, can diminish expression of a particular target protein by the cell. Accordingly, functional nucleic acid can be administered to decrease expression of a particular protein in a subject in need thereof.

Transgene delivery vectors also find use in diagnostic and screening methods, whereby a nucleic acid of interest is transiently or stably expressed in a transgenic animal model.

The transgene delivery vectors can also be used for various non-therapeutic purposes, including but not limited to use in protocols to assess gene targeting, clearance, transcription, translation, etc., as would be apparent to one skilled in the art. The transgene delivery vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

The present invention further comprises a kit or kits to carry out the methods of this invention. A kit of this invention can comprise reagents, buffers, and apparatus for mixing, measuring, sorting, labeling, etc., as well as instructions and the like.

In some embodiments, the invention provides a kit for comprising one or more CEMs of the invention, and/or expression cassettes and/or vectors and/or cells comprising the same as described herein, with optional instructions for the use thereof. In some embodiments, a kit may further comprise a CRISPR-Cas guide nucleic acid (corresponding to an engineered protein, which may be encoded by a polynucleotide of the invention) and/or expression cassettes and/or vectors and or cells comprising the same.

### Pharmaceutical Formulations, Subjects, and Modes of Administration

Provided according to embodiments of the invention are compositions that include a transgene delivery vector. Also provided herein are pharmaceutical compositions comprising a transgene delivery vector in a pharmaceutically acceptable carrier and, optionally, other medicinal agents, pharmaceutical agents, stabilizing agents, buffers, carriers, adjuvants, diluents, etc. In addition to the transgene delivery vector, the fusion protein and/or CEM of the invention may be present in the same pharmaceutical composition as the transgene delivery vector or in separate pharmaceutical compositions. For injection, the carrier will typically be a liquid. For other methods of administration, the carrier may be either solid or liquid. For inhalation administration, the carrier will be respirable, and optionally can be in solid or liquid particulate form. By “pharmaceutically acceptable” it is meant a material that is not toxic or otherwise undesirable, i.e., the material may be administered to a subject without causing any undesirable biological effects.

A further aspect of the invention is a method of administering the delivery vector, fusion protein, gRNA, a protein with a DNA binding domain that binds to the target gene sequence and the gRNA targeting polynucleotide sequence and/or CEM to subjects. A further aspect of the invention is a method of administering the delivery vector, fusion protein and/or CEM to subjects.

Administration of the delivery vectors, fusion proteins, and/or CEMs to a human subject or an animal in need thereof can be by any means known in the art. Optionally, the transgene delivery vector, fusion protein, and/or CEM is delivered in a treatment effective or prevention effective dose in a pharmaceutically acceptable carrier.

Injectables can be prepared in conventional forms, either as liquid solutions or suspensions, solid forms suitable for solution or suspension in liquid prior to injection, or as emulsions. Alternatively, one may administer the transgene delivery vectors, fusion proteins, and/or CEMs in a local rather than systemic manner, for example, in a depot or sustained-release formulation. Further, the transgene delivery vectors, fusion proteins, and/or CEMs can be delivered adhered to a surgically implantable matrix (e.g., as described in U.S. Patent Publication No. 2004-0013645). The delivery vectors, fusion proteins, and/or CEMs disclosed herein can be administered to the lungs of a subject by any suitable means, optionally by administering an aerosol suspension of respirable particles comprised of the delivery vectors, fusion proteins, and/or CEMs, which the subject inhales. The respirable particles can be liquid or solid. Aerosols of liquid particles comprising the transgene delivery vectors, fusion proteins, and/or CEMs may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the delivery vectors, fusion proteins, and/or CEMs may likewise be produced with any solid particulate medicament aerosol generator, by techniques known in the pharmaceutical art.

In certain embodiments, the delivery vectors, fusion proteins, and/or CEMs are administered to a subject in need thereof as early as possible in the life of the subject, e.g., as soon as the subject is diagnosed with a disease or disorder. In some embodiments, the methods are carried out on a newborn subject, e.g., after newborn screening has identified a disease or disorder. In some embodiments, methods are carried out on a subject prior to the age of 10 years, e.g., prior to 1, 2, 3, 4, 5, 6, 7, 8, 9, or 10 years of age. In some embodiments, the methods are carried out on juvenile or adult subjects after the age of 10 years. In some embodiments, the methods are carried out on a fetus in utero, e.g., after prenatal screening has identified a disease or disorder. In some embodiments, the methods are carried out on a subject as soon as the subject develops symptoms associated with a disease or disorder. In some embodiments, the methods are carried out on a subject before the subject develops symptoms associated with a disease or disorder, e.g., a subject that is suspected or diagnosed as having a disease or disorder but has not started to exhibit symptoms.

The delivery vectors, fusion proteins, and/or CEMs may be administered to a subject by any route of administration found to be effective to regulate transgene expression in the host cell. The most suitable route will depend on the subject being treated and the disorder or condition being treated. In some embodiments, the delivery vectors, fusion proteins, and/or CEMs are administered to the subject by a route selected from oral, rectal, transmucosal, intranasal, inhalation (e.g., via an aerosol), buccal (e.g., sublingual), vaginal, intrathecal, intraocular, intravitreal, intracochlear, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye (e.g., by intrastromal, topical, intracameral, intravitreal, subconjunctival, suprachoroidal, sub-Tenon, retrobulbar, or subretinal administration), skeletal muscle, cardiac muscle, diaphragm muscle or brain (e.g., by intrathecal, intracerebral, intraventricular, intranasal, intra-aural, intra-ocular, or peri-ocular delivery administration)).

In particular embodiments, more than one administration (e.g., two, three, four or more administrations) may be employed to achieve the desired level of gene expression over a period of various intervals, e.g., daily, weekly, monthly, yearly, etc.

The delivery vectors, fusion proteins, and/or CEMs can be administered to tissues of the CNS (e.g., brain, eye) and may advantageously result in broader distribution of the delivery vectors, fusion proteins, and/or CEMs than would be observed in the absence of the present invention.

Administration can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye.

Administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The delivery vectors, fusion proteins, and/or CEMs can be delivered to skeletal muscle by intravenous administration, intra-arterial administration, intraperitoneal administration, limb perfusion, (optionally, isolated limb perfusion of a leg and/or arm; see, e.g., Arruda et al., (2005) Blood 105:3458-3464), and/or direct intramuscular injection. In particular embodiments, the delivery vectors, fusion proteins, and/or CEMs are administered to a limb (arm and/or leg) of a subject (e.g., a subject with muscular dystrophy such as DMD) by limb perfusion, optionally isolated limb perfusion (e.g., by intravenous or intra-articular administration. In embodiments of the invention, the delivery vectors, fusion proteins, and/or CEMs can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the agent to cross the endothelial cell barrier. In particular embodiments, the delivery vectors, fusion proteins, and/or CEMs can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Administration to cardiac muscle includes administration to the left atrium, right atrium, left ventricle, right ventricle and/or septum. The delivery vectors, fusion proteins, and/or CEMs can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Administration to smooth muscle can be by any suitable method including intravenous administration, intra-arterial administration, and/or intra-peritoneal administration. In one embodiment, administration can be to endothelial cells present in, near, and/or on smooth muscle.

Delivery to a target tissue can also be achieved by delivering a depot comprising the delivery vectors, fusion proteins, and/or CEMs. In representative embodiments, a depot comprising the delivery vectors, fusion proteins, and/or CEMs is implanted into skeletal, smooth, cardiac and/or diaphragm muscle tissue or the tissue can be contacted with a film or other matrix comprising the heterologous agent. Such implantable matrices or substrates are described in U.S. Pat. No. 7,201,898.

Administration can also be to a tumor (e.g., in or near a tumor or a lymph node). The most suitable route in any given case will depend on the nature and severity of the condition being treated and/or prevented and on the nature of the particular vector that is being used.

The delivery vectors, fusion proteins, and/or CEMs may be delivered or targeted to any tissue or organ in the subject. The target tissue or organ may be in vivo or ex vivo (e.g., corneas or other tissues for transplantation). In some embodiments, the delivery vectors, fusion proteins, and/or CEMs are administered to, e.g., a skeletal muscle, a smooth muscle, the heart, the diaphragm, the airway epithelium, the liver, the kidney, the spleen, the pancreas, the skin, the lung, the ear, and the eye. In some embodiments, the delivery vectors, fusion proteins, and/or CEMs are administered to a diseased tissue or organ, e.g., a tumor.

In general, the delivery vectors of the present invention can be employed to deliver a nucleic acid encoding a polypeptide or functional nucleic acid to treat and/or prevent any disease state for which it is beneficial to deliver a therapeutic polypeptide or functional nucleic acid. Illustrative disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic defects, congenital emphysema (al-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1), serca2a, zinc finger proteins that regulate the phospholamban gene, Barkct, B2-adrenergic receptor, β2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). In one example, HLA-G isoforms may be administered. As another example, bone morphogenic proteins (including BNP 2, 7, etc., RANKL and/or VEGF) can be administered with a bone allograft, for example, following a break or surgical removal in a cancer patient.

In particular embodiments, a delivery vectors are administered to skeletal muscle, diaphragm muscle and/or cardiac muscle (e.g., to treat and/or prevent muscular dystrophy or heart disease [for example, PAD or congestive heart failure]).

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, delivery vectors permit the treatment and/or prevention of genetic diseases.

As a further aspect, the delivery vectors of the present invention may be used to produce an immune response in a subject. According to this embodiment, delivery vectors comprising a nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the delivery vectors may be administered to a cell ex vivo and the altered cell is administered to the subject. The delivery vectors comprising the nucleic acid is introduced into the cell, and the cell is administered to the subject, where the nucleic acid encoding the immunogen can be expressed and induce an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof. In particular embodiments, the nucleic acid delivery vector or cell comprising the nucleic acid can be administered in an immunogenically effective amount, as described below.

The delivery vectors can also be administered for cancer immunotherapy by administration of delivery vectors expressing one or more cancer cell antigens (or an immunologically similar molecule) or any other immunogen that produces an immune response against a cancer cell. To illustrate, an immune response can be produced against a cancer cell antigen in a subject by administering delivery vectors comprising a nucleic acid encoding the cancer cell antigen, for example to treat a patient with cancer and/or to prevent cancer from developing in the subject. The delivery vectors may be administered to a subject in vivo or by using ex vivo methods, as described herein. Alternatively, the cancer antigen can be expressed as part of the delivery vectors.

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In particular embodiments, cells may be removed from a subject with cancer and contacted with delivery vectors. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, y-interferon, @-interferon, t-interferon, interleukin-1a, interleukin-1ß, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-«, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the delivery vectors.

Cytokines may be administered by any method known in the art. Exogenous cytokines may be administered to the subject, or alternatively, a nucleic acid encoding a cytokine may be delivered to the subject using a suitable vector, and the cytokine produced in vivo.

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a polynucleotide including those described herein. As a further option, the subject can be a laboratory animal and/or an animal model of disease. Preferably, the subject is a human.

In some embodiments, the delivery vectors are introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

The delivery vectors can further be administered to elicit an immunogenic response (e.g., as a vaccine). Typically, immunogenic compositions of the present invention comprise an immunogenically effective amount of delivery vectors in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the delivery vectors to be administered to a subject depend upon the mode of administration, the disease or condition to be treated and/or prevented, the individual subject's condition, the particular delivery vector, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ transducing units, optionally about 10⁸-10¹⁵ transducing units.

In a representative embodiment, the invention provides a method of treating and/or preventing muscular dystrophy in a subject in need thereof, the method comprising: administering a treatment or prevention effective amount of delivery vectors to a mammalian subject, wherein the delivery vector comprises a nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the delivery vectors can be administered to skeletal, diaphragm and/or cardiac muscle as described elsewhere herein.

Alternatively, the invention can be practiced to deliver a nucleic acid to skeletal, cardiac or diaphragm muscle, which is used as a platform for production of a polypeptide (e.g., an enzyme) or functional nuclei acid (e.g., functional RNA, e.g., RNAi, microRNA, antisense RNA) that normally circulates in the blood or for systemic delivery to other tissues to treat and/or prevent a disorder (e.g., a metabolic disorder, such as diabetes (e.g., insulin), hemophilia (e.g., Factor IX or Factor VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid α-glucosidase] or Fabry disease [α-galactosidase A]) or a glycogen storage disorder (such as Pompe disease [lysosomal acid α glucosidase]). Other suitable proteins for treating and/or preventing metabolic disorders are described above. The use of muscle as a platform to express a nucleic acid of interest is described in U.S. Patent Publication No. 2002/0192189.

Thus, as one aspect, the invention further encompasses a method of treating and/or preventing a metabolic disorder in a subject in need thereof, the method comprising: administering a treatment or prevention effective amount of delivery vectors to a subject (e.g., to skeletal muscle of a subject), wherein the delivery vector comprises a nucleic acid encoding a polypeptide, wherein the metabolic disorder is a result of a deficiency and/or defect in the polypeptide. Illustrative metabolic disorders and nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). Without being limited by any particular theory of the invention, according to this embodiment, administration to the skeletal muscle can result in secretion of the polypeptide into the systemic circulation and delivery to target tissue(s). Methods of delivering heterologous agent and the cell membrane fusion protein or a functional fragment or derivative thereof to skeletal muscle are described in more detail herein.

The invention can also be practiced to produce antisense RNA, RNAi or other functional RNA (e.g., a ribozyme) for systemic or local delivery.

The invention also provides a method of treating and/or preventing congenital heart failure or PAD in a subject in need thereof, the method comprising administering a treatment or prevention effective amount of delivery vectors to a mammalian subject, wherein the heterologous agent comprises a nucleic acid encoding, for example, a sarcoplasmic endoreticulum Ca²⁺-ATPase (SERCA2a), an angiogenic factor, phosphatase inhibitor I (I-1), RNAi against phospholamban; a phospholamban inhibitory or dominant-negative molecule such as phospholamban S16E, a zinc finger protein that regulates the phospholamban gene, β2-adrenergic receptor, β2-adrenergic receptor kinase (BARK), PI3 kinase, calsarcan, a β-adrenergic receptor kinase inhibitor (BARKct), inhibitor 1 of protein phosphatase 1, S100A1, parvalbumin, adenylyl cyclase type 6, a molecule that effects G-protein coupled receptor kinase type 2 knockdown such as a truncated constitutively active bARKct, Pim-1, PGC-1a, SOD-1, SOD-2, EC-SOD, kallikrein, HIF, thymosin-β4, mir-1, mir-133, mir-206 and/or mir-208.

In particular embodiments, the delivery vectors may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of delivery vectors encoding one or more neurotrophic factors.

Age-related macular degeneration involves both angiogenesis and retinal degeneration. This disorder can be treated by administering delivery vectors encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the delivery vectors. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using delivery vectors of the invention to treat a pituitary tumor. According to this embodiment, the delivery vectors encoding somatostatin (or an active fragment thereof) are administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art.

In particular embodiments, the delivery vectors can comprise a secretory signal as described in U.S. Pat. No. 7,071,172.

In representative embodiments of the invention, the delivery vectors are administered to the CNS (e.g., to the brain or to the eye). The delivery vectors may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and inferior colliculus. The delivery vectors may also be administered to different regions of the eye such as the retina, cornea and/or optic nerve.

The delivery vectors may be delivered into the cerebrospinal fluid (e.g., by lumbar puncture) for more disperse administration of the delivery vectors. The delivery vectors may further be administered intravascularly to the CNS in situations in which the blood-brain barrier has been perturbed (e.g., brain tumor or cerebral infarct).

The delivery vectors can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the delivery vectors are administered in a liquid formulation by direct injection (e.g., stereotactic injection) to the desired region or compartment in the CNS. In other embodiments, the delivery vectors may be provided by topical application to the desired region or by intra-nasal administration of an aerosol formulation. Administration to the eye, may be by topical application of liquid droplets. As a further alternative, the delivery vectors may be administered as a solid, slow-release formulation (see, e.g., U.S. Pat. No. 7,201,898).

In yet additional embodiments, the delivery vectors can used for retrograde transport to treat and/or prevent diseases and disorders involving motor neurons (e.g., amyotrophic lateral sclerosis (ALS); spinal muscular atrophy (SMA), etc.). For example, the delivery vectors can be delivered to muscle tissue or parts of the eye (e.g., anterior ocular segment or eyelid-associated glands) from which it can migrate into neurons.

### Methods of Modulating Expression of a Gene

One aspect of the invention related to methods of modulating expression of a target gene, the method comprising: contacting a polynucleotide comprising a target gene sequence with: 1) a fusion protein comprising a FK506-binding protein (FKBP) polypeptide with a F36V mutation and a gRNA binding polypeptide that binds a gRNA; 2) a gRNA comprising a gene targeting polynucleotide sequence that binds to a target gene sequence and a polynucleotide sequence capable of binding (recognized by) the gRNA binding polypeptide; 3) a CEM of the present invention; and 4) a protein with a DNA binding domain that binds to the target gene sequence and the gRNA targeting polynucleotide sequence, thereby modulating expression of the target gene.

In one aspect, a method of modulating expression of a target gene in a subject comprises administering to the subject: a fusion protein comprising a FK506-binding protein (FKBP) polypeptide with a F36V mutation that binds a CEM and a gRNA binding polypeptide that binds gRNA; a gRNA comprising a gene targeting polynucleotide sequence that binds to a target gene sequence and a polynucleotide sequence capable of binding (recognized by) the gRNA binding polypeptide; a protein with a DNA binding domain that binds to the target gene sequence and the gRNA targeting polynucleotide sequence; and a CEM of the present invention, thereby modulating expression of the gene.

In some embodiments, administering the one or more of the fusion proteins can comprise administering a vector encoding the protein. In an embodiment, the delivery vector can comprise polynucleotides encoding one or more of a fusion protein comprising a FK506-binding protein (FKBP) polypeptide with a F36V mutation and a gRNA binding polypeptide, a gRNA comprising a gene targeting polynucleotide sequence that binds to a target gene sequence and a polynucleotide sequence capable of binding (recognized by) the gRNA binding polypeptide, a protein with a DNA binding domain that binds to the target gene sequence and the gRNA targeting polynucleotide sequence.

A further aspect of the invention relates to methods of treating a disorder that is treatable by modulating expression of a gene in a subject in need thereof, the method comprising: administering to the subject: a fusion protein comprising a FK506-binding protein (FKBP) polypeptide with a F36V mutation that binds a CEM and a gRNA binding polypeptide that binds gRNA; a gRNA comprising a gene targeting polynucleotide sequence that binds to a target gene sequence in the gene to be modulated and a protein binding polynucleotide sequence that binds to the gRNA binding protein polypeptide; a protein with a DNA binding domain that binds to the target gene sequence and the gRNA targeting polynucleotide sequence; and a CEM of the invention, thereby treating the disorder. In an embodiment, the fusion protein binds the CEM and the gRNA. In an embodiment, the gRNA forms a complex with the protein with the DNA binding domain at the target gene sequence. In an embodiment, the CEM bound to the fusion protein also binds the chromatin regulatory protein, bringing the chromatin regulatory protein in proximity to the target gene to thereby modulate expression of the target gene.

In some embodiments of the methods, the fusion protein is provided separately from the delivery vector. For example, a delivery vector and the fusion protein may be separately delivered into a cell or to a subject. In some embodiments, the fusion protein may already be in the cell, e.g., because the cell expresses the fusion protein.

In other embodiments, the polynucleotide further comprises a sequence encoding the fusion protein. The nucleic acid binding domain recognition sequence may be any nucleotide sequence that is specifically recognized and bound by a nucleic acid binding protein such that the presence of the nucleic acid binding domain recognition sequence in the delivery vector recruits a fusion protein comprising the nucleic acid binding protein, as described elsewhere herein.

In some embodiments, modulating expression of a gene or transgene comprises increasing expression of the gene or transgene by contacting the gene or transgene delivery vector with the CEM or administering the CEM to the subject to increase expression of the transgene or gene. In certain embodiments, the CEM binds to chromatin regulatory protein, e.g., a transcriptional activator protein or complex that when recruited to the gene or transgene delivery vector increases expression of the gene or transgene.

In some embodiments, modulating expression of a gene or transgene comprises decreasing expression of the gene or transgene which may comprise contacting the gene or transgene delivery vector with the CEM or administering the CEM to the subject to decrease expression of the gene or transgene. In certain embodiments, the CEM binds to a chromatin regulatory protein, e.g., a transcriptional repressor protein or complex that when recruited to the gene or transgene delivery vector decreases expression of the gene or transgene. In an aspect, reversibility of the increased or decreased expression is achieved with removal of the CEM.

### Methods of Modulating Transgene Expression

One aspect of the invention relates to methods of modulating expression of a transgene from a transgene delivery vector, the method comprising: providing a transgene delivery vector comprising a polynucleotide comprising a transgene expression cassette and a nucleic acid binding domain recognition sequence; contacting the transgene delivery vector with a fusion protein comprising a nucleic acid binding domain that binds to the recognition sequence fused to a domain that binds a chemical epigenetic modifier; and contacting the transgene delivery vector with the chemical epigenetic modifier; thereby modulating expression of the transgene from the transgene delivery vector.

Another aspect of the invention relates to methods of modulating expression of a transgene from a transgene delivery vector in a subject, the method comprising: administering to the subject a transgene delivery vector comprising a polynucleotide comprising a transgene expression cassette and a nucleic acid binding domain recognition sequence; administering to the subject a fusion protein comprising a nucleic acid binding domain that binds to the recognition sequence fused to a domain that binds a chemical epigenetic modifier; and administering to the subject the chemical epigenetic modifier; thereby modulating expression of the transgene.

A further aspect of the invention relates to methods of treating a disorder that is treatable by expression of a transgene from a transgene delivery vector in a subject in need thereof, the method comprising: administering to the subject a transgene delivery vector comprising a polynucleotide comprising a transgene expression cassette and a nucleic acid binding domain recognition sequence; administering to the subject a fusion protein comprising a nucleic acid binding domain that binds to the recognition sequence fused to a domain that binds a chemical epigenetic modifier; and administering to the subject the chemical epigenetic modifier; thereby treating the disorder.

The transgene delivery vector may be any type of vector known to be useful for delivering a polynucleotide to a cell. In some embodiments, the transgene delivery vector is a viral vector, e.g., a viral genome. Examples of viral vectors include, without limitation, an adeno-associated virus, retrovirus, lentivirus, poxvirus, alphavirus, baculovirus, vaccinia virus, herpes virus, Epstein-Barr virus, or adenovirus vector.

In some embodiments, the transgene delivery vector is a non-viral vector. Examples of non-viral vectors include, without limitation, a plasmid, liposome, electrically charged lipid, nucleic acid-protein complex, or biopolymer.

In some embodiments of the methods, the fusion protein is provided separately from the transgene delivery vector. For example, a transgene delivery vector and the fusion protein may be separately delivered into a cell or to a subject. In some embodiments, the fusion protein may already be in the cell, e.g., because the cell expresses the fusion protein.

In other embodiments, the polynucleotide further comprises a sequence encoding the fusion protein, e.g., so that the transgene-encoded product and the fusion protein are both produced by the transgene delivery vector. The nucleic acid binding domain recognition sequence may be any nucleotide sequence that is specifically recognized and bound by a nucleic acid binding protein such that the presence of the nucleic acid binding domain recognition sequence in the transgene delivery vector recruits a fusion protein comprising the nucleic acid binding protein, as described elsewhere herein.

In some embodiments, modulating expression of a transgene comprises increasing expression of the transgene and contacting the transgene delivery vector with the CEM or administering the CEM to the subject increases expression of the transgene. In certain embodiments, the CEM binds to a transcriptional activator protein or complex that when recruited to the transgene delivery vector increases expression of the transgene. Examples of a transcriptional modulator protein or complex include, without limitation, BRD4, HDAC, or CBP/p300.

Having described the present invention, the same will be explained in greater detail in the following examples, which are included herein for illustration purposes only, and which are not intended to be limiting to the invention.

### EXAMPLES

### Example 1

The inventors describe Chemical Epigenetic Modifiers (CEMs), a class of bifunctional molecules which can recruit endogenous chromatin machinery to achieve dose-dependent regulation of gene expression. The system incorporates a dCas9 that binds an engineered single guide RNA (gRNA) containing a MS2 stem loop that can bind to MS2 coating proteins (MCPs). By using a MS2-FKBPx2 fusion, the CEM molecules can be efficiently located at targeted gene sites. CEM87 is the best-in-class activating CEM (CEMa) molecule, composed of FK506 and I-BET762, which binds to dCas9-FKBP-based gene targeting protein complexes and recruits endogenous BET proteins, respectively.

The CEM technology is dependent on FK506 binding to FKBP to locate CEM molecules to the targeted DNA sequence. FK506 was used due to the ease of creating these reagents synthetically. However, in eukaryotic cells, FKBP has multiple functions and, therefore, FKBP-based CEMs may have potential off-target effects resulting from endogenous FKBP binding, leading to higher treatment dosages and reduced efficacy. In an effort to improve the specificity and efficacy of the CEM technology, a mutant F36V-FKBP (FKBP*) and the corresponding synthetic ligand of FKBP* was investigated. The Holt lab published a set of novel compounds that possess substituents designed to favor binding to FKBP* and sterically reduce binding affinity to wild type FKBP (WT-FKBP), and demonstrated their selectivity for FKBP* over WT-FKBP. FKBP* and the corresponding bumped synthetic ligand of FKBP* (SLF*) have proven to be useful in several biological techniques including live cell imaging and microarray screening methodology development (Yang, W. et al. Investigating Protein-Ligand Interactions with a Mutant FKBP Possessing a Designed Specificity Pocket. J. Med. Chem. 43, 1135-42 (2000)). More recently, the Bradner lab appended CRBN ligands to AP1867, (an SLF*), and successfully induced degradation of an FKBP* protein fusion. FK506 was replaced with AP1867 in the second generation CEMs (SLF*-CEMs) to eliminate confounding biological effects of inhibiting endogenous WT-FKBP and decrease the effective dose of our CEMs (Nabet, B. et al. The dTAG system for immediate and target-specific protein degradation. Nat. Chem. Biol. 14, 431-441 (2018)). Herein, the SLF*-CEM activator (SLF*-CEMa) is described, a bioorthogonal technology coupled with CRISPR-Cas9 to achieve dose-dependent gene specific regulation. This chemically-based gene activating platform can be coupled with other chemical induced proximity (CIP) systems, adding to the toolkit for synthetic biologists.

### Chemical Design and Validation of SLF*-CEMa in a Reporter System

For this proof-of-concept work, 12 SLF*-CEMa molecules that recruit BET bromodomain proteins (BRD2, BRD3, and BRD4) to targeted gene sites via the SLF* ligand, AP1867, were synthesized for initial testing (FIG. 1A, FIG. 1B). Two sets of compounds incorporating different BET ligands were designed to establish the preferred BRD recruiting ligand, with CEM201 through CEM206 containing (+)-JQ1 and CEM207 through CEM212 containing I-BET762. Utilizing the acid handles of (+)-JQ1-COOH, I-BET762-COOH, and AP1867, a variety of PEGₙ linkers (n=2-7) were installed via amide couplings to explore various linker lengths between the bifunctional CEM components. In the context of PROTACs, linker length and composition can be critical in dictating the formation of a E3:PROTAC:POI ternary complex and subsequent degradation. However, an analogous analysis has not been done for CEMs until now.

To screen the activation activity of newly synthesized SLF*-CEMa molecules, a green fluorescent protein (GFP) reporter system was transfected downstream of a TRE3G promoter, into a human colorectal carcinoma cell line, HCT116, together with dCas9, MS2-FKBP*x2, and TRE3G gRNA or non-targeting gRNA (NT gRNA) as a negative control group. After treatment with the 12 SLF*-CEMa molecules at 10 nM for 48 hours, flow cytometry was performed and mean fluorescence values were collected (FIG. 5A). All SLF*-CEMa compounds showed strong GFP activation with no increase of GFP expression observed in the NT gRNA groups. This demonstrated that replacing FK506 with AP1867 in the CEMs does not compromise the gene activation capability, but rather enables the development of a potentially bioorthogonal CEMa system. Additionally, this suggests that both (+)-JQ1 and I-BET762 effectively recruit the BET bromodomains in the context of the SLF*-CEMa molecules and that multiple linker lengths are well tolerated.

The SLF*-CEMa system was further evaluated by conducting a control experiment to test if either the BET ligands or AP1867 result in any non-specific activation themselves. HCT116 cells were transfected with the same GFP reporter system used above and followed with the treatment of vehicle DMSO, AP1867, I-BET762-COOH, AP1867 and I-BET762-COOH together, and one of the SLF*-CEMa (CEM202). All final compound concentrations were kept in cell media at 10 nM in both the TR3G gRNA group and NT gRNA groups. After 48 hours of compound exposure, flow cytometry was performed and the mean fluorescent GFP value was collected (FIG. 1C). GFP activation was only observed in the group treated with CEM202 and TRE3G gRNA, which demonstrates that gene activation was not caused by the individual components of the SLF*-CEMa molecules.

Next, to verify that the SLF*-CEMa compounds are functioning through the binding of SLF* to FKBP*, a competition assay was conducted in the same TRE3G-GFP system in HCT116 cells treated with 5 nM CEM207 and increasing concentrations of AP1867. No change in GFP expression was observed with up to 0.5 μM of AP1867 (FIG. 5B). Increasing the concentration of AP1867 to 5 μM, 10 μM, and 20 μM resulted in a 45%, 84%, and 91% decrease in CEM207-mediated GFP activation, respectively, which verifies that gene activation is achieved via the binding of FKBP* to SLF* (FIG. 1D).

Having systematically verified that SLF*-CEMa cause GFP activation by functioning as a bifunctional molecule, a dose-response experiment was performed with two of the top performing SLF*-CEMa, CEM202 containing I-BET762 and CEM207 containing (+)-JQ1 (FIG. 1B), to further evaluate their efficacy. To investigate if an FK506-containing CEM could promote GFP expression in the FKBP* system, first-generation CEM87 side-by-side data was also included. After 48 hours, dose-dependent GFP activation was observed by flow cytometry (FIG. 1E). Both I-BET762 and (+)-JQ1 warhead-containing SLF*-CEMa showed strong GFP activation, with CEM207 activating GFP expression at picomolar concentrations; a 2.0-fold change at 0.25 nM and 9.1-fold change at 0.5 nM CEM207 (compared to vehicle control) were observed. CEM202 also showed strong activation in the low nanomolar range, with 2.8-fold activation at 2.5 nM and 12.5-fold activation at 5 nM. CEM207 demonstrated maximum GFP activation at 5 nM (24.0-fold) and CEM202 at 50 nM (26.7-fold). No significant activation was achieved upon CEM87 treatment until the concentration was increased to 50 nM, which is in agreement with the weak binding affinity of FK506 to FKBP*; however, the extent of activation was still much less than that observed with CEM202 and CEM207 at this concentration. Likewise, all NT gRNA control cells had no significant GFP activation in the dose-response experiment (FIG. 5C).

To better understand the activity of SLF*-CEMa in the WT FKBP-based GFP reporter assay, a similar dose-response assay was conducted in HCT116 cells transfected with dCas9 and MS2-FKBPx2 (FIGS. 1D and 1E). No activation was observed with either SLF*-CEMa at the doses where maximum activation was achieved in the F36V-FKBP-based system. Thus, the orthogonality of the SLF*-based CEMs were thoroughtly validated to first-generation WT FKBP-based system.

### Dose-Dependent Activation of Endogenous Genes by SLF*-CEMa

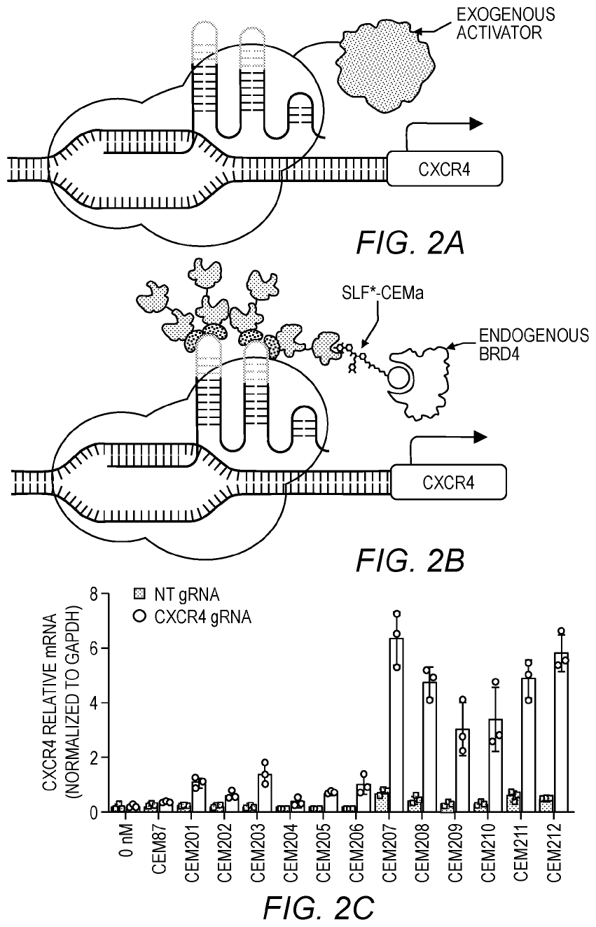

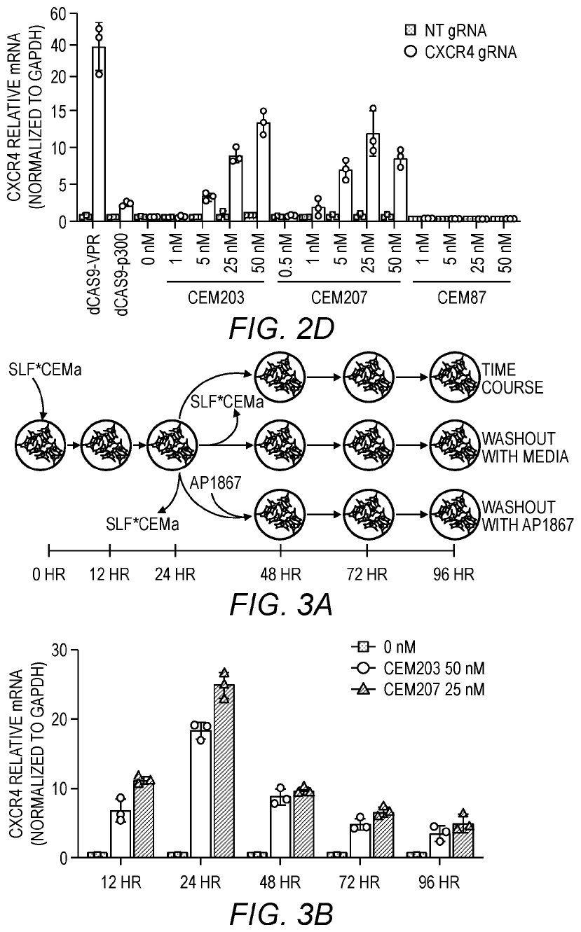

To further explore the activation potential of the SLF*-CEMa, their ability to target endogenous genes was evaluated (FIGS. 2A and 2B). Lentivirus was used to establish a stable HCT116 cell line expressing dCas9 and MS2-FKBP*x2 constructs, selecting for cells with antibiotic resistance. CXCR4 was chosen as the endogenous gene target for initial tests, as successful activation of this gene has been demonstrated in HCT116 cells with FK506-based CEMs (FIG. 2B). The same multiplex CXCR4 gRNA system was adapted due to the high activation activity. All CEMs were screened for their ability to activate the CXCR4 gene at 5 nM (FIG. 2C). RNA extraction and quantitative RT-PCR (qRT-PCR) were performed after 48 hours of CEM treatment. According to the mRNA expression, all 12 SLF*-CEMa molecules successfully activated CXCR4 expression as compared to the NT gRNA control. In general, SLF*-CEMa molecules containing (+)-JQ1 (CEM207-CEM212) demonstrated stronger activation activity compared to SLF*-CEMa molecules with I-BET762 (CEM201-CEM206) as the recruitment ligand. Furthermore, different linker lengths led to overall moderate differences in activation indicating the tolerance of a variety of linker lengths. This is in contrast to other bifunctional molecules such as PROTACs where small changes in linker composition can drastically influence PROTAC efficacy. Based on the mRNA expression data, CEM203 (containing I-BET762) and CEM207 (containing (+)-JQ1) was chosen to conduct further experiments, which showed the best activity in each warhead group.

To determine the optimal treatment concentration of the SLF*-CEMa molecules in this context, HCT116 cells were treated with 1 nM, 5 nM, 25 nM, and 50 nM of CEM203, CEM207, or CEM87. Cells with 0.5 nM of CEM207 were also treated based on the picomolar activity observed in the previous GFP reporter assay. After 48 hours, RNA extraction followed by qRT-PCR was performed to evaluate the change in mRNA expression of CXCR4 (FIG. 2D). Compared to the no chemical treated control, CEM203 and CEM207 reached peak activation activity at 50 nM (54.0-fold change) and 25 nM (48.2-fold change), respectively, while no significant activation of CXCR4 mRNA expression was observed at all concentrations of CEM87 tested. It is worth noting that 28.2-fold CXCR4 activation was achieved with the incredibly low dose of 5 nM of CEM207, demonstrating the high potency of the SLF*-CEMa compounds, despite their large size. To directly compare this induced activation to other CRISPR-Cas9-based epigenetic activators, dCas9-VPR and dCas9-p300 constructs were virally infected into HCT116 cells and CXCR4 activation assessed in a similar fashion (FIGS. 2A and 2B). In this experiment, dCas9-VPR activated CXCR4 to an extremely high level. Compared to dCas9-p300, both CEM203 and CEM207 achieved higher CXCR4 activation at concentrations above 5 nM (FIG. 2D), which further demonstrates that SLF*-CEMa is a powerful gene activating technology.

To verify the orthogonality of the SLF*-CEMa and WT-FKBP in an endogenous system, lentivirally infected HCT116 cells were used which stably express dCas9 and MS2-FKBPx2 constructs. After transfecting CXCR4 gRNA, the cells were treated with the same concentrations of CEM203, CEM207, and CEM87 as were used previously, and RNA extraction was performed after 48 hours (FIG. 6). As expected, no significant activation was observed upon treatment with 50 nM of CEM203 and 25 nM of CEM207 in the WT-FKBP system, again demonstrating that SLF*-CEMa molecules have reduced affinity for WT-FKBP. Overall, the SLF*-CEMa can serve as potent activators of the endogenous target gene CXCR4 in a dose-dependent manner while eliminating confounding biological effects of inhibiting endogenous WT-FKBP.

### Time Dependence and Reversibility of SLF*-CEMa Gene Activation

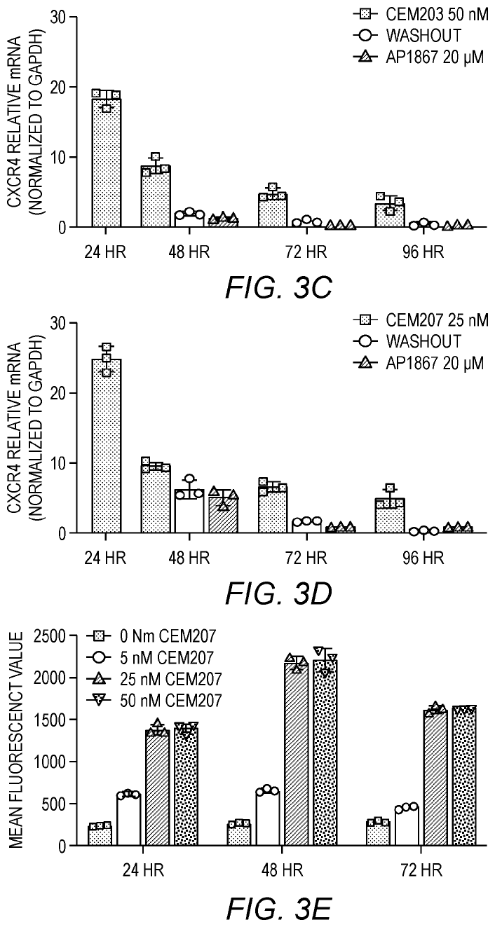

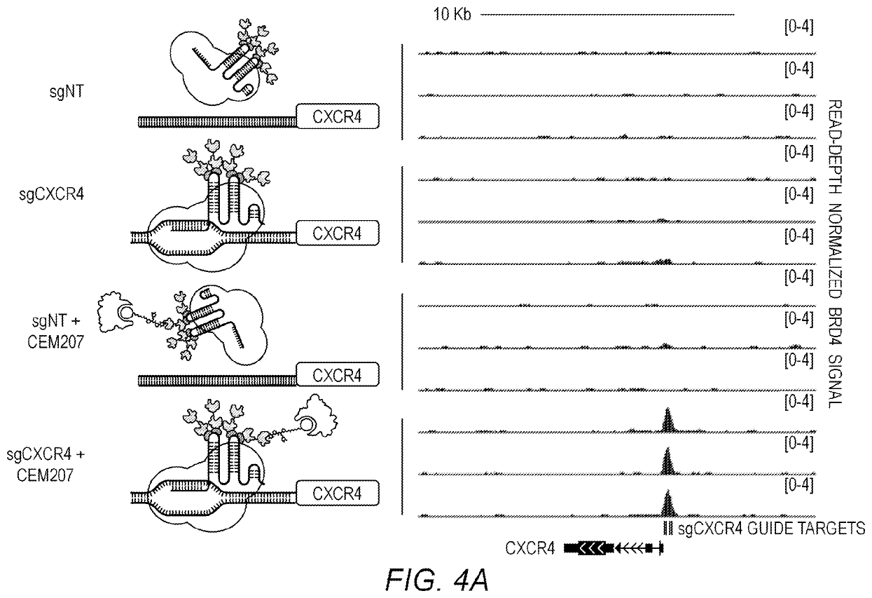

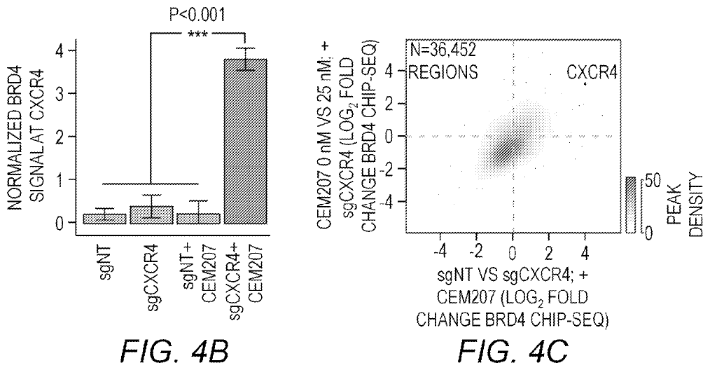

To determine the time at which SLF*-CEMa most effectively induces gene activation, CXCR4 gene expression changes over time were investigated in dCas9 and MS2-FKBP*x2 stably infected HCT116 cells. Cells were treated with either vehicle, 25 nM CEM203 or 50 nM CEM207 (FIG. 3A). RNA extraction and qRT-PCR were performed at 12, 24, 48, 72, and 96 hrs time points (FIG. 3B). Gene activation was observed as early as 12 hrs in both CEM203 and CEM207 treated groups. At the 24 hrs time point, CEM203 and CEM207 showed the highest CXCR4 activation. Notably, the activation levels were reduced after 24 hrs, likely due to dilution of gRNA plasmids as a result of cell division. However, CXCR4 had significant activation compared to the NT gRNA control after 96 hours with both CEM203 and CEM207, demonstrating the long-lasting activity of the SLF*-CEMa molecules.

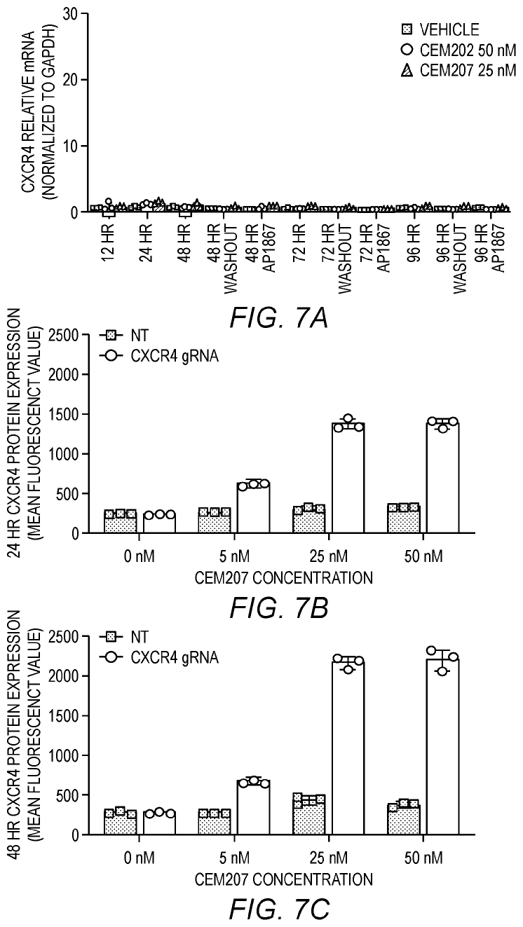

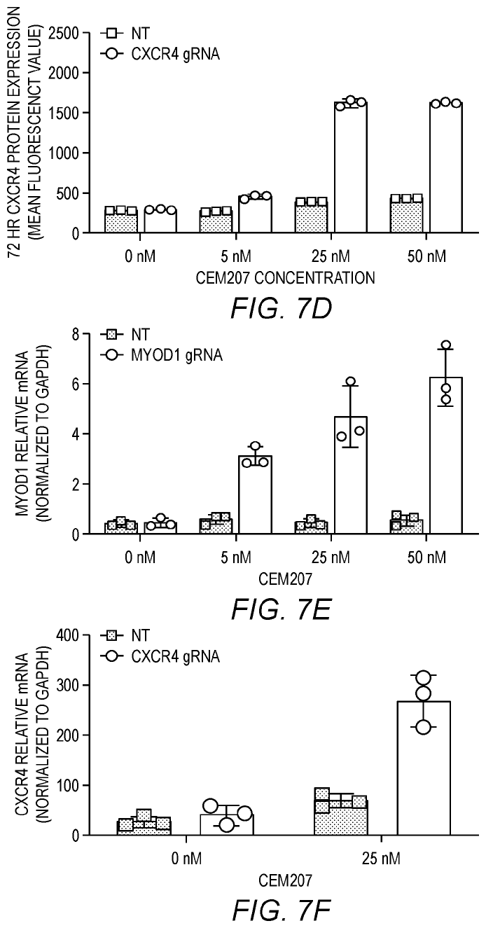

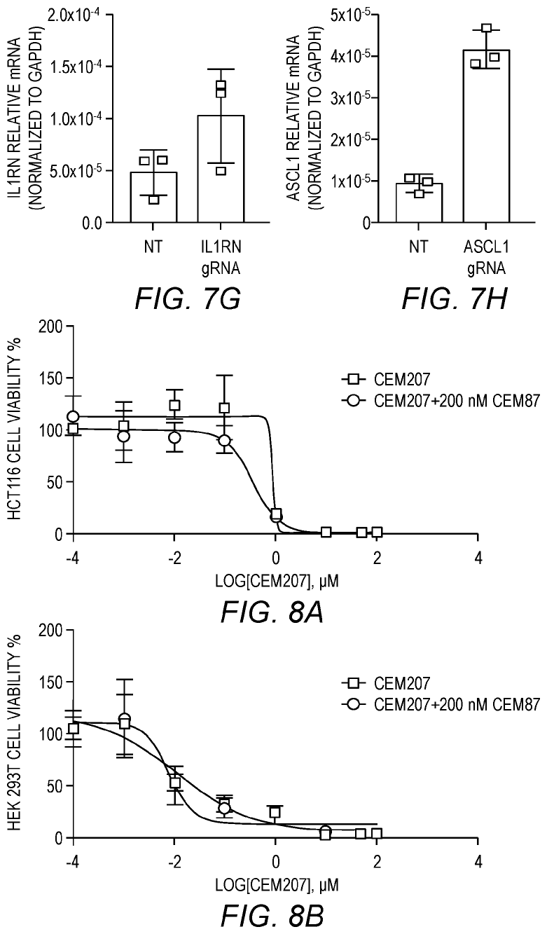

The reversibility of FK506-CEMa induced gene activation was demonstrated in the WT-FKBP reporter system. To investigate if endogenous gene activation triggered by the SLF*-CEMa compounds is also reversible, HCT116 cells were treated with CEM203 (50 nM) or CEM207 (25 nM) for 24 hours after transfecting CXCR4 gRNA, and then the CEMs were removed by media exchange at various time points. In comparison to the cells continuously treated with CEMs, CEM washout significantly lowered CXCR4 mRNA expression at all time points (FIGS. 3C and 3D), demonstrating that SLF*-CEMa activation can be reversed upon CEM removal. Likewise, it was investigated whether treatment with excess AP1867 could further reverse gene activation, potentially in a more robust fashion than with washout alone if there is any remaining intracellular SLF*-CEMa. To test this, after 24 hours of SLF*-CEMa treatment, a media exchange containing 20 μM AP1867 was performed (FIGS. 3C and 3D). As hypothesized, the activation of CXCR4 expression in AP1867 treated cells with CEM washout was the lowest among all three treatments. At 72 hours, no significant activation was detected, further supporting that gene activation is dependent on the binding of FKBP* to SLF* and demonstrating that the rate of reversal of gene activation is enhanced in the presence of competing FKBP* ligand AP1867. No significant gene activation was detected in all NT gRNA control groups (FIG. 7A).

In addition, the change in CXCR4 protein expression was evaluated following CEM207 treatment at three different concentrations. CEM207 was chosen over CEM203 because of the lower concentration needed to reach a similar peak CXCR4 mRNA activation level. An APC anti-CXCR4 antibody was used to assess cell membrane CXCR4 protein expression via flow cytometry in dCas9 and MS2-FKBP*x2 stably infected HCT116 cells (FIG. 3E and FIGS. 7B-7D). CXCR4 protein levels were determined at 24, 48, and 72 hrs time points for each of the different concentrations of CEM207. Maximum CXCR4 cell membrane protein expression was observed at 25 nM CEM207 treatment and remained the same at 50 nM. Whereas maximum gene activation was observed 24 hours after CEM treatment based on mRNA levels, CXCR4 protein expression was highest 48 hours after CEM treatment, which is expected given the timing of protein translation following transcription. Based on this protein expression data, it was concluded that the SLF*-CEMa technology can achieve effective gene-specific, dose-dependent activation at the mRNA and protein level.

### Benchmarking SLF*-CEMa System in Different Cell Lines and at Different Genes

As a gene-specific activation technology, it is important to investigate the universal applicability of SLF*-CEMa and evaluate their ability to activate variable genes in different tissue cells. To do so, HEK293T cells stably expressing dCas9 and MS2-FKBP*x2 constructs via lentiviral infection were established and benchmarked for the gene activation level of MYOD1, CXCR4, and IL1RN after CEM207 treatment for 24 hours. HEK293T cells transfected with MYOD1 gRNA were treated with 5 nM, 25 nM, or 50 nM of CEM207. RNA extraction and qRT-PCR were preformed after 24 hours. Significant activation in all CEM207 treated cells was observed with a dose-dependent increase in MYOD1 mRNA expression (FIG. 7E). In the corresponding CXCR4 and IL1RN gene activation experiments, CEM207 showed different activation activity on these two genes. CXCR4 was significantly activated at 25 nM after 24 hours of treatment (FIG. 7F), while IL1RN activation appeared to be subtle and not statistically significant at the same CEM207 concentration and treatment time (FIG. 7G). As a result, another gene was tested in the HCT116 cell line. Stably infected cells were transfected with ASCL1 gRNA and mRNA expression was measured by qRT-PCR. Under the same CEM207 treatment conditions (25 nM, 24 h), ASCL1 expression increased significantly (FIG. 7H). Given that different genes in different tissues have variable activation potentials, in some cases the SLF*-CEMa do not result in significant activation; however, overall, it was observed that they promote activation of different genes in multiple cell lines in a dose-dependent manner.

### Genome-Wide BRD4 Enrichment

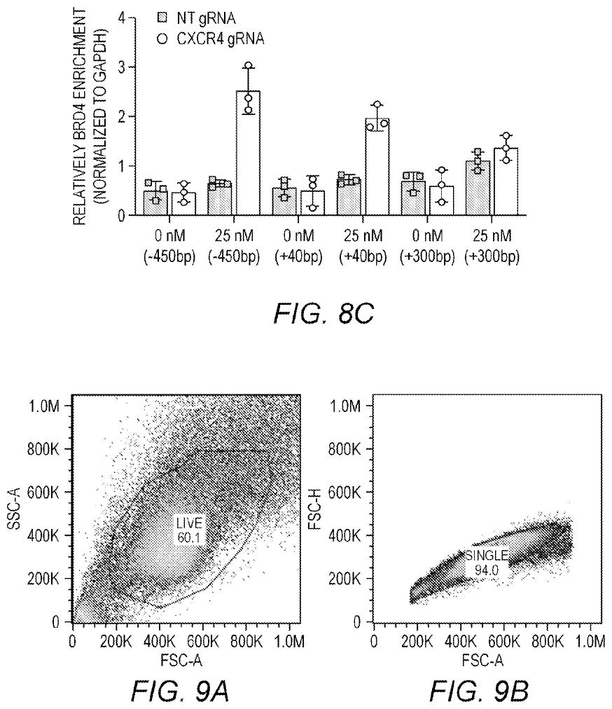

Given that SLF*-CEMa facilitates gene activation via recruitment of BET proteins including BRD4, BRD4 enrichment at a specific targeted gene locus was investigated to further validate the mechanism of CEM207. Based on the alamarBlue cell viability assay performed with HEK293T and HCT116 cells, CEM207 can have different toxicity effects on different cell lines after 4 days of exposure. Specifically, HCT116 appeared to be more tolerant to CEM207 treatment. Here, the IC₅₀ values of CEM207 alone was 0.870 μM and CEM207 in combination with FK506-based compound CEM87 was tested. The two-compound combination had an IC₅₀ value of 0.359 μM for CEM207 in the background of 200 nM CEM87 (FIG. 8A). HEK293T cells were less tolerant of CEM207 treatment based on the lower IC₅₀ values observed in the same alamarBlue assay (IC₅₀=106 nM with CEM207 treatment alone and 78.6 nM when treated simultaneously with 200 nM CEM87) (FIG. 8B). Importantly, CEM207 can promote efficient gene activation at concentrations well below these IC₅₀ values where little to no toxicity is observed after 24 h treatment. Based on the cell viability data, chromatin immunoprecipitation-qPCR (ChIP-qPCR) was performed in stably infected HCT116 cells. Cells were transfected with CXCR4 gRNA and treated with 25 nM CEM207 for 24 hours. qPCR primer sets priming at different sites near the CXCR4 gene transcription start site (TSS) were used to capture BRD4 localization. At multiple locations just upstream of the TSS, BRD4 enrichment was observed by ChIP-qPCR (FIG. 8A). To demonstrate the robustness and specificity of SLF*-CEMa technology, ChIP-seq for BRD4 was performed under four conditions: no CEM+sgNT, no CEM+sgCXCR4, 25 nM CEM207+sgNT, and 25 nM CEM207+sgCXCR4. BRD4 ChIP-seq mimicked the observations in the ChIP-qPCR experiment, revealing a well-defined BRD4 signal peak at the CXCR4 locus (FIG. 8A) that was only discernable when both CEM207 and gRNA were present (FIG. 8B). To assess any off-target effects, a union set of peaks was created across all samples and conditions. The signal change was measured between samples when only CEM207 or gRNA are present and those where both are present. Most BRD4 peaks were remarkably stable between conditions, with 96.8% of peaks having a log₂ fold-change in signal less than two in either direction. To determine outlier significance, a “z-score” distribution was calculated by taking the scaled means of the x- and y-axis values and calculated P values for each point within this distribution. This revealed that the CXCR4 peak had the highest z-score and was a significant outlier (adjusted P value=2.62×10^{-0.5}, FIG. 8C). Therefore, both the ChIP-qPCR and ChIP-seq results strongly support that the gene specific activation observed in the dCas9-based SLF*-CEMa system is achieved via the accumulation of endogenous epigenetic machinery at the designed gRNA targeted gene locus.

Herein, an improved Chemical Epigenetic Modifier technology is described which utilizes a mutated version of FKBP, F36V-FKBP, in a DNA binding complex and the corresponding ligand, AP1867. This approach makes two important advances. First, it reduces the interactions between endogenous WT-FKBP and the CEM molecules which could lead to off target effects. Second, this next generation of CEMs works orthogonally to the original FK506-based CEMs, potentially allowing for temporal regulation of two different genes or sets of genes simultaneously. Furthermore, the new bump-hole SLF*-CEMa can effectively promote gene activation at remarkably low doses in the low nanomolar range. It is also demonstrated that the effects of the SLF*-CEMa molecules are reversible over time, potentially allowing for an added level of control of gene expression. Importantly, this technology arms synthetic biologists with a bioorthagonal chemical based approach to regulate the mammalian genome using a CRISPR DNA targeting system.

### Methods

Chemical Synthesis. See Example 2 for details. CEM molecules were diluted in DMSO (Sigma, D2650) and stored at −20° C.

Statistical analysis. All significance of flow cytometry and qPCR data was determined by Student's t-test (two tailed). Error bars represent the standard deviations of three different cell culture replicates. Further information is included in Table 2.

Plasmid design. The S. pyogenes dCas9-MS2 compatible gRNA plasmids were modified from Addgene plasmid 61427. The multiplexed MS2 stem-loop containing gRNAs were modified from Addgene kit 1000000055 to be expressed in lentiviral backbone and remove the Cas9 protein. Specific gRNA sequences for each gene can be found in Table 3.

Cell culture. HCT116 cells were cultured in McCoy's 5A media (Corning, 10-050-CV) with 10% FBS (R&D Systems) and penicillin-streptomycin (Gibco, 15140-122). HEK293T cells were cultured in high-glucose DMEM (Corning, 10-013-CV), supplemented with 10% FBS (Atlanta Biologicals, S10250), 10 mM HEPES (Corning, 25-060-CI), NEAA (Gibco, 11140-050), 55 μM of 2-mercaptoethanol (Gibco, 21985-023), and penicillin-streptomycin (Gibco, 15140-122). Cells were passaged every 2-5 days and maintained at 20%-90% confluency in 37° C. and 5% CO₂.

Cell transfection. Cells were transfected on the next day after splitting with polyethylenimine (PEI; Polysciences, 23966-1), DNA, and Opti-MEM (Gibco, 31985070) at 3:1:100 ratio (μl:μg:μl). The media was changed 16 h later.

Flow cytometry. All flow cytometry experiments were performed with Attune Nxt as previously described. CXCR4 protein assay was done using a fluorophore-conjugated primary antibody (anti-CXCR4-APC; BioLegend, 306510) following the protocol described previously²⁸. Significance was determined by Student's t-test.

Lentiviral infection. Lentivirus used in HEK293T and HCT116 cells were produced by Lenti-X 293T cells (Clontech). Each lentivirus was produced in 15-cm plates with 18 μg of the packaging construct plasmid, 13.5 μg of Gag-Pol (Addgene, 12260), and 4.5 μg of VSV-G (Addgene, 12259). Plasmid transfection was done with PEI. After 60 hrs, the cell media was collected and spun down at 20,000 r.p.m. for 2.5 hrs at 4° C. The supernatant was removed, and the virus pellet was resuspended with 1×PBS before being added to the cells needed to be infected. 10 μg ml⁻¹ Polybrene (Santa, Cruz Biotechnology, sc-134220) was used along with the virus to increase the infection efficacy.

RNA extraction and qRT-PCR. All RNA extraction experiments were conducted in 12-well plate format. Culturing medium was removed, and cells were washed with 1×PBS, following with 0.05% trypsin to disassociate. After being quenched by medium and centrifuged, cells were washed with 1×PBS, and the RNA extraction was performed by using RNeasy Plus Mini kit (Qiagen, 74134). RT-qPCR was conducted by using RNA-to C_{T} 1-step kit (Thermo Fisher Scientific, 4389986). The primer sets used in quantifying each gene can be found in Table 4.

AlamarBlue cell viability assay. HEK293T or HCT116 cells were placed in black 96-well plates with clear bottom. 24 hrs later, media was exchanged to include 0.001, 0.01, 0.1, 1, 10, 50, 100 μM concentrations of CEM207, or all CEM207 concentrations above along with 200 nM CEM87. After 4 days of chemical exposure, alamarBlue Cell Viability Reagent (Invitrogen, DAL1025) was added to each well. Cells were incubated at 37° C. for 4 hrs and fluorescence was read using the TECAN Infinite F200 Fluorescence Microplate Reader. IC₅₀ values were calculated using GraphPad Prism 9.

ChIP-qPCR. ChIP-qPCR was done exactly as previously described. BRD4 was enriched with Rb mAB to Brd4 (abcam, ab 128874). qPCR Primer sets can be found in Table 4.

ChIP-seq library preparation. Chromatin bound to BRD4 was immunoprecipitated from fixed and sheared nuclei, with or without CEM207 treatment, in HCT116 cells bearing sgNT or sgCXCR4 (promoter). Enriched DNA from ChIP was barcoded and libraries were amplified, followed by size selection (fragment ranges between 250 and 1,000 bp), using NEBNext® Ultra™ II DNA Library Prep with Sample Purification Beads (E7103S) and NEBNext® Multiplex Oligos for Illumina® (96 Unique Dual Index Primer Pairs) (E6440S). Libraries were sequenced to a depth of 30 million base pairs (75-bp single-end reads) on an Illumina NextSeq 500.

ChIP-seq analyses. Reads from the sequencer were demultiplexed using bcl2fastq (v2.20.0). Sequencing adapters on reads were trimmed using cutadapt (v1.12) using options -a GATCGGAAGAGC (SEQ ID NO: 35) and—minimum-length 36 in paired mode. After trimming, reads were filtered for quality using the fastq_quality_filter in FASTX-Toolkit (v0.0.12), with options -Q 33, -p 90, and -q 20. In-house scripts were used to limit potential PCR duplicates by limiting reads with the same sequence to a maximum of five copies and discarding the copies beyond that limit. Once de-duplicated, read alignment was done using STAR (v2.5.2b) and options —outFilterMismatchNmax 2, —chimSegmentMin 15, —chimJunctionOverhangMin 15, —outSAMtype BAM Unsorted, —outFilterType BySJout, —outFilterScoreMin 1, and —outFilterMultimapNmax 1. Post-alignment, SAMtools (v1.13) and BEDTools (2.26) (were used to generate bigWig files for downstream analyses. BRD4 signal was read depth normalized (per million mapped reads). MACS2 was used to call BRD4 peaks on each individual replicate using the input controls and default settings. BRD4 windows were created by taking the peak summits and adding 500 base pairs in both directions to create equal sized regions for even comparisons in subsequent analyses.

To create a union set of peaks, the “score per million” (SPM) method was used first described in Corces et al., Science, 362:6413 (2018); doi: 10.1126/science.aav1898. As sample quality or read depth increases, the number of peaks and their significance scores also increase which makes comparing peaks between samples difficult. To control for this, SPM creates relative peak (or region) scores for each sample that can more directly be compared. In brief, within each sample the per-region “score per million” was calculated by taking the individual peak score (−log (10)*adjusted p-value) and dividing by the total sum of all peak scores divided by one million. Every region in every sample was then collected to make a cumulative union region set. When overlapping regions were found, only the region with the highest SPM value was kept. deepTools (v2.5.4) was used to calculate the mean BRD4 signal per region.

Density-scaled scatterplots were made using the smoothScatter function in R (v3.3.1) with the option nrpoints=50 to highlight the 50 most outlying points. Points were calculated by taking the log 2 fold changes between the mean signal per region across replicates for each sample. To calculate point p-values, a “z-score” was calculated taking the means of the x- and y-values for each point. In R, the “z-scores” were used in the pnorm function to generate p-values while p.adjust was used to Benjamini-Hochberg correct them.

### Abbreviations

CEM: Chemical Epigenetic Modifier
FKBP: FK506 binding protein
PTM: post-translational modification
HAT: histone acetyl transferase
HDAC: histone deacetylase
ZF: Zinc Finger
TALE: transcription activator-like effector
PROTAC: Proteolysis targeting chimera
POI: protein-of-interest
gRNA: guide RNA
MCP: MS2 coating protein
CEMa: activating CEM
FKBP*: F36V-FKBP
CRBN: ubiquitin E3 ligase cereblon
SLF*: Synthetic ligand of F36V-FKBP
SLF*-CEMa: SLF*-CEM activator
CIP: chemical induced proximity
GFP: green fluorescent protein
NT: non-targeting
qRT-PCR: quantitative RT-PCR
ChIP: chromatin immunoprecipitation
TSS: Transcription start site

TABLE 2

| | fold | |
|---|---|---|
| | change | p value |
| FIG. 1C (TRE3G gRNA | | |
| vs NT gRNA) | | |
| 0 nM | 1.7 | 0.0554240 |
| AP1867 | 1.4 | 0.1279630 |
| iBET762 | 2.4 | 0.0027360 |
| iBET762 + AP1867 | 1.5 | 0.1279630 |
| CEM202 | 29.5 | 0.0027360 |
| FIG. 1D (no AP1867 | | |
| vs AP1867 added) | | |
| TRE3G gRNA | (% repress) | |
| CEM207 5 nM + | 45% | 0.001628 |
| AP1867 5 μM | | |
| CEM207 5 nM + | 84% | 0.000126 |
| AP1867 10 μM | | |
| CEM207 5 nM + | 91% | 8.91E−05 |
| AP1867 20 μM | | |
| NT gRNA | | |
| CEM207 5 nM + | 21% | 0.019246 |
| AP1867 5 μM | | |
| CEM207 5 nM + | 23% | 0.006371 |
| AP1867 10 μM | | |
| CEM207 5 nM + | 29% | 0.002669 |
| AP1867 20 μM | | |
| FIG. 1E (Treated | | |
| vs Non-Treated) | | |
| CEM202 0.25 nM | 1.1 | 0.160591 |
| CEM202 0.5 nM | 1.3 | 0.004805 |
| CEM202 0.75 nM | 1.3 | 0.006501 |
| CEM202 1 n M | 1.5 | 0.000343 |
| CEM202 2.5 nM | 2.8 | 4.5E−07 |
| CEM202 5 nM | 12.5 | 1.73E−06 |
| CEM202 7.5 nM | 13.9 | 2.27E−05 |
| CEM202 10 nM | 15.0 | 0.000311 |
| CEM202 25 nM | 22.0 | 2.64E−07 |
| CEM202 50 nM | 28.8 | 1.3E−05 |
| CEM202 75 nM | 27.1 | 6.01E−06 |
| CEM202 100 nM | 25.5 | 3.24E−08 |
| CEM207 0.25 nM | 2.0 | 0.001241 |
| CEM207 0.5 nM | 9.1 | 3.99E−06 |
| CEM207 0.75 nM | 11.5 | 9.28E−07 |
| CEM207 1 n M | 12.8 | 1.24E−05 |
| CEM207 2.5 nM | 20.6 | 9.98E−06 |
| CEM207 5 nM | 24.0 | 2.45E−05 |
| CEM207 7.5 nM | 22.5 | 4.21E−06 |
| CEM207 10 nM | 21.4 | 8.55E−08 |
| CEM207 25 nM | 21.5 | 1.15E−05 |
| CEM207 50 nM | 22.3 | 1.62E−07 |
| CEM207 75 nM | 22.5 | 1.26E−05 |
| CEM207 100 nM | 21.9 | 2.62E−05 |
| CEM87 0.25 nM | 1.1 | 0.056075 |
| CEM87 0.5 nM | 1.1 | 0.02525 |
| CEM87 0.75 nM | 1.1 | 0.080983 |
| CEM87 1 n M | 1.1 | 0.049153 |
| CEM87 2.5 nM | 1.2 | 0.048807 |
| CEM87 5 nM | 1.1 | 0.014807 |
| CEM87 7.5 nM | 1.1 | 0.011634 |
| CEM87 10 nM | 1.2 | 0.015579 |
| CEM87 25 nM | 1.6 | 0.000606 |
| CEM87 50 nM | 5.2 | 1.92E−05 |
| CEM87 75 nM | 7.8 | 8.47E−05 |
| CEM87 100 nM | 9.7 | 2.15E−05 |
| FIG. 2C (Treated | | |
| vs Non-Treated) | | |
| 0 nM | 1.3 | 0.607491 |
| CEM87 | 1.5 | 0.063678 |
| CEM201 | 5.8 | 0.001408 |
| CEM202 | 3.5 | 0.005848 |
| CEM203 | 11.4 | 0.004802 |
| CEM204 | 7.2 | 0.037264 |
| CEM205 | 8.8 | 1.66E−05 |
| CEM206 | 13.9 | 0.01083 |
| CEM207 | 8.9 | 0.000603 |
| CEM208 | 11.5 | 0.000238 |
| CEM209 | 11.1 | 0.008201 |
| CEM210 | 12.0 | 0.010747 |
| CEM211 | 8.5 | 0.000553 |
| CEM212 | 12.6 | 0.000156 |
| FIG. 2D (CEM Treated | | |
| vs Non-Treated) | | |
| CXCR4 gRNA | | |
| dCas9-VPR | 156.4 | 0.000551 |
| dCas9-p300 | 9.5 | 0.012319 |
| CEM203 1 nM | 2.3 | 0.10124 |
| CEM203 5 nM | 13.0 | 0.000478 |
| CEM203 25 nM | 35.9 | 0.000118 |
| CEM203 50 nM | 54.0 | 0.00014 |
| CEM207 0.5 nM | 2.5 | 0.045324 |
| CEM207 1 nM | 7.4 | 0.087974 |
| CEM207 5 nM | 28.2 | 0.00093 |
| CEM207 25 nM | 48.2 | 0.00258 |
| CEM207 50 nM | 34.6 | 0.000296 |
| CEM87 1 nM | 1.16 | 0.530753 |
| CEM87 5 nM | 1.03 | 0.864411 |
| CEM87 25 nM | 1.19 | 0.318639 |
| CEM87 50 nM | 1.53 | 0.113117 |
| NT gRNA | | |
| dCas9-VPR | 1.42 | 0.461618 |
| dCas9-p300 | 0.84 | 0.683873 |
| CEM203 1 nM | 0.98 | 0.9672 |
| CEM203 5 nM | 1.30 | 0.474974 |
| CEM203 25 nM | 2.42 | 0.159508 |
| CEM203 50 nM | 2.57 | 0.011425 |
| CEM207 0.5 nM | 1.17 | 0.768289 |
| CEM207 1 nM | 0.77 | 0.548828 |
| CEM207 5 nM | 1.47 | 0.355645 |
| CEM207 25 nM | 2.21 | 0.107504 |
| CEM207 50 nM | 1.88 | 0.172244 |
| CEM87 1 nM | 0.85 | 0.715472 |
| CEM87 5 nM | 0.81 | 0.64979 |
| CEM87 25 nM | 0.91 | 0.804958 |
| CEM87 50 nM | 1.18 | 0.67514 |
| FIG. 3B (CXCR4 | | |
| gRNA vs NT gRNA) | | |
| 0 nM groups | | |
| 12 h | 1.1 | 0.9889860 |
| 24 h | 1.3 | 0.7041340 |
| 48 h | 1.3 | 0.9274610 |
| 48 h wash | 1.5 | 0.0186950 |
| 48 h AP1867 | 1.0 | 0.9924840 |
| 72 h | 0.9 | 0.9924840 |
| 72 h wash | 0.9 | 0.9924840 |
| 72 h AP1867 | 0.9 | 0.9892540 |
| 96 h | 0.9 | 0.9924840 |
| 96 h wash | 2.2 | 0.8520780 |
| 96 h AP1867 | 0.5 | 0.0739690 |
| CEM203 50 nM groups | | |
| 12 h | 9.6 | 0.020679 |
| 24 h | 17.9 | 0.000188 |
| 48 h | 19.8 | 0.002836 |
| 48 h wash | 7.7 | 0.002836 |
| 48 h AP1867 | 3.6 | 0.02157 |
| 72 h | 13.1 | 0.006936 |
| 72 h wash | 5.6 | 0.009352 |
| 72 h AP1867 | 1.9 | 0.098349 |
| 96 h | 12.8 | 0.141271 |
| 96 h wash | 3.3 | 0.141271 |
| 96 h AP1867 | 1.2 | 0.711061 |
| CEM207 25 nM groups | | |
| 12 h | 16.8 | 0.000059 |
| 24 h | 18.6 | 0.000248 |
| 48 h | 13.1 | 0.000248 |
| 48 h wash | 10.5 | 0.008238 |
| 48 h AP1867 | 8.3 | 0.008238 |
| 72 h | 6.8 | 0.002462 |
| 72 h wash | 5.4 | 0.001997 |
| 72 h AP1867 | 4.2 | 0.008014 |
| 96 h | 7.3 | 0.011042 |
| 96 h wash | 4.3 | 0.003019 |
| 96 h AP1867 | 2.1 | 0.027397 |
| FIG. 3C (washout vs | | |
| AP1867 competition) | | |
| 48 h | 1.5 | 0.024654 |
| 72 h | 4.2 | 0.001724 |
| 96 h | 1.7 | 0.2362945 |
| FIG. 3D (washout vs | | |
| AP1867 competition) | | |
| 48 h | 1.2 | 0.3083446 |
| 72 h | 2.4 | 0.0014026 |
| 96 h | 0.57 | 0.0496582 |
| FIG. 3E (CXCR4 | | |
| gRNA vs NT gRNA) | | |
| 24 h time point | | |
| 0 nM | 0.9 | 0.110321 |
| 5 nM | 2.4 | 0.000062 |
| 25 nM | 4.6 | 0.000038 |
| 50 nM | 4.3 | 0.000026 |
| 48 h time point | | |
| 0 nM | 1.0 | 0.454631 |
| 5 nM | 2.4 | 0.000066 |
| 25 nM | 4.9 | 0.000024 |
| 50 nM | 5.8 | 0.000066 |
| 72 h time point | | |
| 0 nM | 1.0 | 0.480699 |
| 5 nM | 1.7 | 0.001311 |
| 25 nM | 4.2 | 0.000003 |
| 50 nM | 3.8 | <0.000001 |
| FIG. 5A | | |
| TRE3G gRNA | | |
| CEM201 | 8.2 | 1.94E−05 |
| CEM202 | 8.4 | 0.000796 |
| CEM203 | 9.4 | 0.028297 |
| CEM204 | 5.7 | 0.038144 |
| CEM205 | 6.9 | 0.002662 |
| CEM206 | 8.5 | 0.011606 |
| CEM207 | 12.0 | 0.014711 |
| CEM208 | 8.1 | 0.008367 |
| CEM209 | 4.3 | 0.004527 |
| CEM210 | 8.5 | 0.027066 |
| CEM211 | 7.8 | 0.009077 |
| CEM212 | 6.1 | 0.000652 |
| NT gRNA | | |
| CEM201 | 0.6 | 0.001379 |
| CEM202 | 0.5 | 0.000928 |
| CEM203 | 0.7 | 0.012713 |
| CEM204 | 0.3 | 0.000819 |
| CEM205 | 0.4 | 0.005697 |
| CEM206 | 0.3 | 0.000871 |
| CEM207 | 0.3 | 0.000502 |
| CEM208 | 0.4 | 0.005949 |
| CEM209 | 0.5 | 0.006502 |
| CEM210 | 0.4 | 0.002991 |
| CEM211 | 0.4 | 0.0039 |
| CEM212 | 0.4 | 0.002427 |
| FIG. 5B (no AP1867 | | |
| vs AP1867 added) | | |
| TRE3G gRNA | | |
| CEM207 5 nM | 1.1 | 0.445056 |
| AP1867 50 nM | | |
| CEM207 5 nM | 1.1 | 0.381143 |
| AP1867 500 nM | | |
| CEM207 5 nM | 0.6 | 0.018117 |
| AP1867 5000 nM | | |
| NT gRNA | | |
| CEM207 5 nM | 1.0 | 0.749625 |
| AP1867 50 nM | | |
| CEM207 5 nM | 1.0 | 0.48965 |
| AP1867 500 nM | | |
| CEM207 5 nM | 0.7 | 0.003936 |
| AP1867 5000 nM | | |
| FIG. 5C (NT gRNA) | | |
| CEM202 0.25 nM | 1.0 | 0.413131 |
| CEM202 0.5 nM | 1.0 | 0.270475 |
| CEM202 0.75 nM | 1.1 | 0.010225 |
| CEM202 1 n M | 1.0 | 0.323592 |
| CEM202 2.5 nM | 1.0 | 0.982285 |
| CEM202 5 nM | 1.1 | 0.161729 |
| CEM202 7.5 nM | 1.1 | 0.019723 |
| CEM202 10 nM | 1.2 | 0.002331 |
| CEM202 25 nM | 1.3 | 0.005036 |
| CEM202 50 nM | 1.4 | 0.000282 |
| CEM202 75 nM | 1.4 | 0.004225 |
| CEM202 100 nM | 1.4 | 7.91E−05 |
| CEM207 0.25 nM | 1.1 | 0.073007 |
| CEM207 0.5 nM | 1.1 | 0.083595 |
| CEM207 0.75 nM | 1.1 | 0.004992 |
| CEM207 1 n M | 1.1 | 0.032108 |
| CEM207 2.5 nM | 1.2 | 0.000269 |
| CEM207 5 nM | 1.5 | 9.33E−05 |
| CEM207 7.5 nM | 1.5 | 7.25E−05 |
| CEM207 10 nM | 1.4 | 0.001082 |
| CEM207 25 nM | 1.6 | 1.85E−05 |
| CEM207 50 nM | 1.6 | 4.67E−06 |
| CEM207 75 nM | 1.5 | 6.91E−06 |
| CEM207 100 nM | 1.5 | 0.000157 |
| CEM87 0.25 nM | 1.1 | 0.10589 |
| CEM87 0.5 nM | 1.1 | 0.036549 |
| CEM87 0.75 nM | 1.0 | 0.858408 |
| CEM87 1 n M | 1.0 | 0.762083 |
| CEM87 2.5 nM | 1.1 | 0.012481 |
| CEM87 5 nM | 1.0 | 0.370104 |
| CEM87 7.5 nM | 1.0 | 0.808865 |
| CEM87 10 nM | 1.0 | 0.147054 |
| CEM87 25 nM | 1.0 | >0.999999999999999 |
| CEM87 50 nM | 1.0 | 0.743303 |
| CEM87 75 nM | 1.0 | 0.838176 |
| CEM87 100 nM | 1.0 | 0.081846 |
| FIG. 5D (Treated | | |
| vs Non-Treated) | | |
| CEM202 10 nM | 0.9 | 0.255969 |
| CEM202 25 nM | 1.0 | 0.196581 |
| CEM202 50 nM | 1.1 | 0.046237 |
| CEM202 100 nM | 5.3 | 4.27E−06 |
| CEM202 200 nM | 11.6 | 3.43E−05 |
| CEM202 400 nM | 17.5 | 1.88E−08 |
| CEM207 10 nM | 1.9 | 0.000211 |
| CEM207 25 nM | 6.5 | 1.13E−05 |
| CEM207 50 nM | 17.4 | 0.000215 |
| CEM207 100 nM | 20.8 | 1E−06 |
| CEM207 200 nM | 24.0 | 2.34E−05 |
| CEM207 400 nM | 27.2 | 1.73E−05 |
| CEM87 10 nM | 22.5 | 3.2E−06 |
| CEM87 25 nM | 23.1 | 2.35E−06 |
| CEM87 50 nM | 22.1 | 1.19E−05 |
| CEM87 100 nM | 22.0 | 2.26E−06 |
| CEM87 200 nM | 18.7 | 2.65E−05 |
| CEM87 400 nM | 18.3 | 3.51E−06 |
| FIG. 5E (Treated | | |
| vs Non-Treated) | | |
| CEM202 10 nM | 0.9 | 0.028395 |
| CEM202 25 nM | 0.9 | 0.066359 |
| CEM202 50 nM | 1.0 | 0.424315 |
| CEM202 100 nM | 1.0 | 0.778823 |
| CEM202 200 nM | 1.0 | 0.32274 |
| CEM202 400 nM | 1.0 | 0.418366 |
| CEM207 10 nM | 1.1 | 0.081658 |
| CEM207 25 nM | 1.1 | 0.072575 |
| CEM207 50 nM | 1.0 | 0.70993 |
| CEM207 100 nM | 1.1 | 0.012314 |
| CEM207 200 nM | 1.2 | 0.005306 |
| CEM207 400 nM | 1.3 | 0.001301 |
| CEM87 10 nM | 1.0 | 0.189297 |
| CEM87 25 nM | 1.1 | 0.009156 |
| CEM87 50 nM | 1.1 | 0.005046 |
| CEM87 100 nM | 1.2 | 0.014684 |
| CEM87 200 nM | 1.1 | 0.083035 |
| CEM87 400 nM | 1.2 | 0.001416 |
| FIG. 6 (CXCR4 | | |
| gRNA vs NT gRNA) | | |
| 0 nM | 1.6 | 0.153923 |
| CEM203 25 nM | 1.5 | 0.477107 |
| CEM203 50 nM | 2.0 | 0.13732 |
| CEM203 200M | 3.5 | 0.00995 |
| CEM207 25 nM | 1.5 | 0.470263 |
| CEM207 50 nM | 4.2 | 0.00213 |
| CEM207 200 nM | 9.1 | 0.014238 |
| CEM87 25 nM | 21.5 | 0.009053 |
| CEM87 50 nM | 16.5 | 0.001758 |
| CEM87 200 nM | 19.0 | 0.004392 |
| FIG. 7A (Compare to 0 h) | | |
| CEM203 50 nM | | |
| 12 h | 4.9 | 0.131631 |
| 24 h | 2.2 | 0.002463 |
| 48 h | 1.1 | 0.899638 |
| 48 h wash | 0.7 | 0.027595 |
| 48 h AP1867 | 0.8 | 0.522032 |
| 72 h | 1.0 | 0.912354 |
| 72 h wash | 0.8 | 0.337495 |
| 72 h AP1867 | 0.6 | 0.185905 |
| 96 h | 1.3 | 0.067046 |
| 96 h wash | 0.7 | 0.293455 |
| 96 h AP1867 | 0.5 | 0.043962 |
| CEM207 25 nM | | |
| 12 h | 0.1 | 0.007467 |
| 24 h | 0.6 | 0.002018 |
| 48 h | 0.7 | 0.325913 |
| 48 h wash | 0.8 | 0.077762 |
| 48 h AP1867 | 0.7 | 0.00701 |
| 72 h | 0.6 | 0.092384 |
| 72 h wash | 0.4 | 0.114854 |
| 72 h AP1867 | 0.3 | 0.921408 |
| 96 h | 0.5 | 0.044182 |
| 96 h wash | 0.6 | 0.250377 |
| 96 h AP1867 | 0.6 | 0.025505 |
| FIG. 7B-7D (CXCR4 | | |
| gRNA vs NT gRNA) | | |
| 24 h | | |
| 0 nM | 0.9 | 0.110321 |
| 5 nM | 2.4 | 0.000062 |
| 25 nM | 4.6 | 0.000038 |
| 50 nM | 4.3 | 0.000026 |
| 48 h | | |
| 0 nM | 1.0 | 0.454631 |
| 5 nM | 2.4 | 0.000066 |
| 25 nM | 4.9 | 0.000024 |
| 50 nM | 5.8 | 0.000066 |
| 72 h | | |
| 0 nM | 1.0 | 0.480699 |
| 5 nM | 1.7 | 0.001311 |
| 25 nM | 4.2 | 0.000003 |
| 50 nM | 3.8 | <0.000001 |
| FIG. 7E (MYOD1 | | |
| gRNA vs NT gRNA) | | |
| 0 nM | 1.1 | 0.669458 |
| 5 nM | 5.4 | 0.000535 |
| 25 nM | 10.8 | 0.003607 |
| 50 nM | 10.9 | 0.001139 |
| FIG. 7F (CXCR4 | | |
| gRNA vs NT gRNA) | | |
| 0 nM | 1.6 | 0.323775 |
| 25 nM | 4.0 | 0.002746 |
| FIG. 7G (IL1RN | | |
| gRNA vs NT gRNA) | | |
| IL1RN gRNA vs NT gRNA | 2.1 | 0.1338 |
| FIG. 7H (ASCL1 | | |
| gRNA vs NT gRNA) | | |
| ASCL1 gRNA vs NT gRNA | 4.4 | 0.0049 |
| FIG. 8 (CXCR4 | | |
| gRNA vs NT gRNA) | | |
| 0 nM | 0.9 | 0.828637 |
| (−450 bp) | | |
| 25 nM | 3.9 | 0.0024 |
| (−450 bp) | | |
| 0 nM | 0.9 | 0.790763 |
| (+40 bp) | | |
| 25 nM | 2.7 | 0.001438 |
| (+40 bp) | | |
| 0 nM | 0.9 | 0.723098 |
| (+300 bp) | | |
| 25 nM | 1.2 | 0.221118 |
| (+300 bp) | | |

TABLE 3
gRNA Sequences

| Target | Protospacer Sequence (5′-3′) |
|---|---|
| TRE3G x6 | TACGTTCTCTATCACTGATA SEQ ID NO: 2 |
| CXCR4 | (1) GCAGACGCGAGGAAGGAGGGCGC SEQ ID NO: 3 |
| multiplexed | (2) AGCCGGACAGGACCTCCCAG SEQ ID NO: 4 |
| | (3) GACCCTGCTGTTTGCGGGTGGT SEQ ID NO: 5 |
| | (4) GAGGGAGAAGGCGGGGTGG SEQ ID NO: 6 |
| IL1RN | (1) GTGTACTCTCTGAGGTGCTC SEQ ID NO: 7 |
| | (2) GACGCAGATAAGAACCAGTT SEQ ID NO: 8 |
| | (3) GCATCAAGTCAGCCATCAGC SEQ ID NO: 9 |
| ASCL1 | (1) GCGGGAGAAAGGAACGGGAGG SEQ ID NO: 10 |
| | (2) GTCCAATTTCTAGGGTCACCG SEQ ID NO: 11 |
| | (3) GAAGAACTTGAAGCAAAGCGC SEQ ID NO: 12 |
| MYOD1 | (1) CCTGGGCTCCGGGGCGTTT SEQ ID NO: 13 |
| multiplexed | (2) GGCCCCTGCGGCCACCCCG SEQ ID NO: 14 |
| | (3) CTCCCTCCCTGCCCGGTAG SEQ ID NO: 15 |
| | (4) AGGTTTGGAAAGGGCGTGC SEQ ID NO: 16 |
| NT | GCGAGGTATTCGGCTCCGCG SEQ ID NO: 17 |

TABLE 4
qPCR primers

| Target | Forward | Reverse |
|---|---|---|
| CXCR4 | ACTACACCGAGGAAATGGGCT | CCCACAATGCCAGTTAAGAAGA |
| | SEQ ID NO: 18 | SEQ ID NO: 19 |
| MYOD1 | CTCTCTGCTCCTTTGCCACA | GTGCTCTTCGGGTTTCAGGA |
| | SEQ ID NO: 20 | SEQ ID NO: 21 |
| GAPDH | CAATGACCCCTTCATTGACC | TTGATTTTGGAGGGATCTCG |
| | SEQ ID NO: 22 | SEQ ID NO: 23 |
| IL1RN | GGAATCCATGGAGGGAAGAT | TGTTCTCGCTCAGGTCAGTG |
| | SEQ ID NO: 24 | SEQ ID NO: 25 |
| ASCL1 | CGC GGC CAA CAA GAA GAT G | CGA CGA GTA GGA TGA GAC CG |
| | SEQ ID NO: 26 | SEQ ID NO: 27 |
| CXCR4 (ChIP) | CTGTGGCGCGTCCATCCTTG | TGGCGTTGGAAGCTTGGCCC |
| −450 bp | SEQ ID NO: 28 | SEQ ID NO: 29 |
| CXCR4 (ChIP) | TCTGCCCGCTCGGAGAGGGG | GCCTGAGTGCTCCAGTAGCCAC |
| +40 bp | SEQ ID NO: 30 | SEQ ID NO: 31 |
| CXCR4 (ChIP) | CATCCCCGCTTCCCCAAGGA | GTGTCTCCCCCTTGAGTCCC |
| +300 bp | SEQ ID NO: 32 | SEQ ID NO: 33 |

MS2-FKBP^{F36V}x2
(SEQ ID NO: 34)
atggcttcaaactttactcagttcgtgctcgtggacaatggtgggacag
gggatgtgacagtggctccttctaatttcgctaatggggtggcagagtg
gatcagctccaactcacggagccaggcctacaaggtgacatgcagcgtc
aggcagtctagtgcccagaagagaaagtataccatcaaggtggaggtcc
ccaaagtggctacccagacagtgggcggagtcgaactgcctgtcgccgc
ttggaggtcctacctgaacatggagctcactatcccaattttcgctacc
aattctgactgtgaactcatcgtgaaggcaatgcaggggctcctcaaag
acggtaatcctatcccttccgccatcgccgctaactcaggtatctacag
cgctggaggaggtggaagcggaggaggaggaagcggaggaggaggtagc
ggacctaagaaaaagaggaaggtggcgggatccgcgggggtccaagttg
aaaccattagtcccggcgatggcaggacttttcctaaaaggggacaaac
atgtgtcgtccattatacaggcatgttggaggacggcaaaaaggtcgac
agtagtagagatcggaataaacctttcaaattcatgttgggaaaacaag
aagtcattaggggatgggaggagggcgtggctcaaatgtccgtcggcca
acgggctaagctcaccatcagccccgactacgcttacggcgctaccggc
catcccggaattatcccgccccacgctaccttggtgtttgacgtcgaat
tgttgaagctcgaactcgagggcgtgcaggtggagactatctccccagg
agacgggcgcaccttccccaagcgcggccagacctgcgtggtgcactac
accgggatgcttgaagatggaaagaaagttgattcctcccgggacagaa
acaagccctttaagtttatgctaggcaagcaggaggtgatccgaggctg
ggaagaaggggttgcccagatgagtgtgggtcagagagccaaactgact
atatctccagattatgcctatggtgccactgggcacccaggcatcatcc
caccacatgccactctcgtcttcgatgtggagcttctaaaactggaata
g

### Example 2

### Chemistry Methods

General chemistry procedures. Reactions were performed in either round-bottom flasks or glass sample vials under ambient conditions with room temperature (rt) generally 25° C. All reagents and solvents were obtained from commercial suppliers and were used without further purification unless otherwise stated. Specifically, I-BET762-COOH, (+)-JQ1, and AP1867 were purchased from MedChemExpress. Thin-layer chromatography (TLC) was performed using commercial silica gel 60 F254-coated glass-backed plates. TLC plates were visualized under 254 nm UV light or by immersion in a basic potassium permanganate solution followed by heating with a heat gun for 30 s. Normal phase flash column chromatography was performed with a Teledyne Isco CombiFlash®Rf using RediSep®Rf silica columns with the UV detector set to 254 nm and 280 nm. Mobile phases A (DCM) and B (MeOH) were used. Reverse phase column chromatography was performed with a Teledyne Isco CombiFlash®Rf using C18 RediSep®Rf Gold columns with the UV detector set to 220 nm and 254 nm. Mobile phases A (H2O+0.1% TFA (v/v)) and B (MeOH or ACN) were used. Preparative HPLC was performed using an Agilent Prep 1200 series with the UV detector set to 220 nm and 254 nm. Samples were injected onto a Phenomenex Luna 75×30 mm (5 μm) C18 column. Mobile phases A (H2O+0.1% TFA) and B (MeOH) were used with a flow rate 30 mL/min.

Analysis of products. Analytical LCMS was used to establish the purity of targeted compounds. All final compounds that were evaluated in biochemical assays had >95% purity as determined by LCMS. Analytical LCMS data for compounds were acquired on an Agilent 6110 Series system with UV detector set to 220 nm, 254 nm, and 280 nm. Samples were injected onto an Agilent ZORBAX Eclipse Plus 4.6×50 mm, 1.8 μm, C18 column at 25° C. Mobile phases A (H2O+0.1% acetic acid) and B (ACN+1% water and 0.1% acetic acid) were used in a linear gradient from 10% to 100% B in 5 min, followed by a flush at 100% B for another 2 min with a flow rate of 1.0 mL/min. Mass spectra (MS) data were acquired in positive ion mode using an Agilent 6110 single quadrupole mass spectrometer with an electrospray ionization (ESI) source. All 1H and 13C nuclear magnetic resonance (NMR) spectra were recorded in deuterated solvent (CD3OD-d4) on a Varian 400 MR NMR spectrometer at 400 MHz. All 13C NMR spectra were recorded in deuterated solvent (CD3OD-d4) on a Varian 400 MR NMR or a Varian Inova 500 NMR spectrometer at 101 MHz and 126 MHz, respectively.

Chemical shifts are reported in parts per million (ppm) and are referenced to residual un-deuterated solvent (CD3OD-d4 referenced at 3.31 ppm for 1H NMR and 49.00 ppm for 13C NMR). Coupling constants are reported in Hertz (Hz) and peaks multiplicities as either a singlet(s), doublet (d), triplet (t), q (quartet), quint (quintet), m (multiplet), dd (doublet of doublets), dt (doublet of triplets), td (triplet of doublets), or br (broad singlet).

### Chemistry Abbreviations

Methanol (MeOH), acetonitrile (ACN), dichloromethane (DCM), ethyl acetate (EtOAc), dimethyl sulfoxide (DMSO), N,N-dimethylformamide (DMF), trifluoroacetic acid (TFA), 2-(1H-benzo[d][1,2,3]triazol-1-yl)-1,1,3,3-tetramethyluronium tetrafluoroborate (TBTU), N-ethyl-N-isopropylpropan-2-amine (DIPEA).

### Synthetic Schemes

### Synthesis of I-BET762-Based CEMs, CEM201 (7)-CEM206 (12)

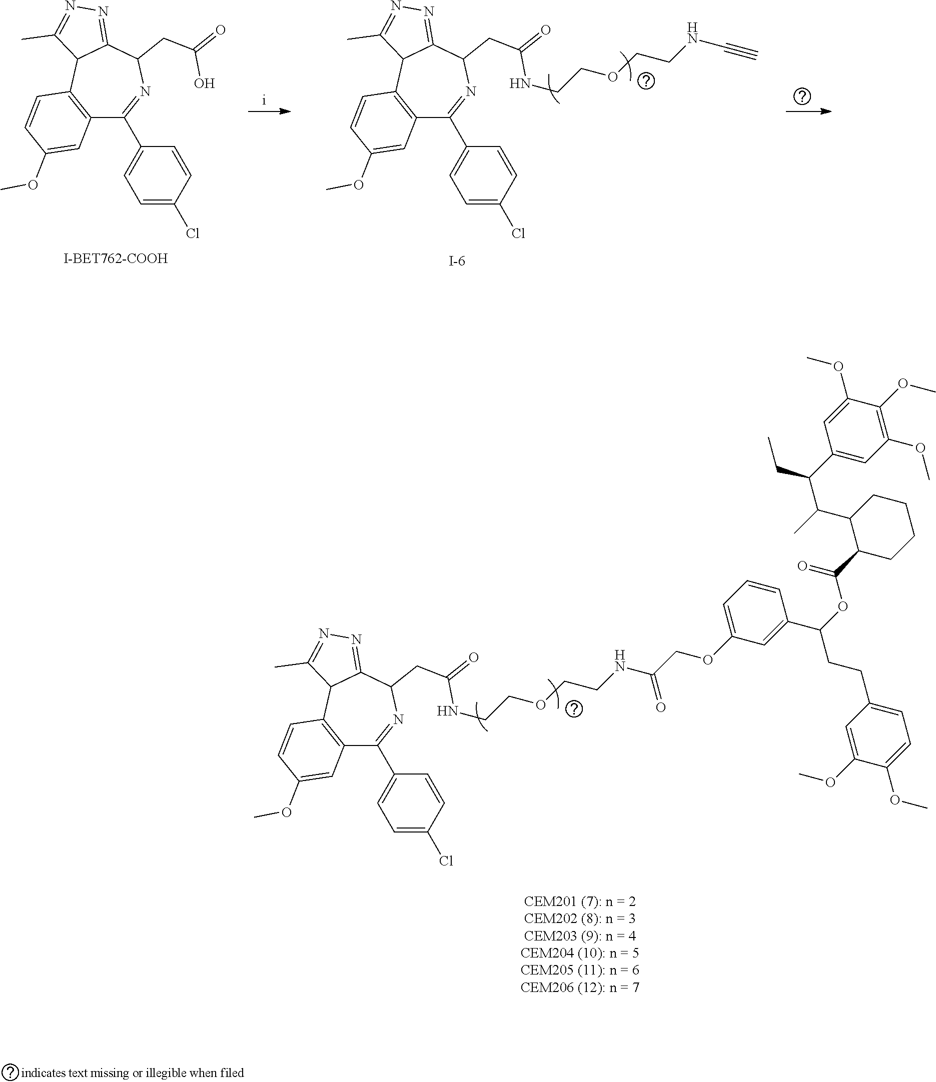

Supplemental scheme 1. Reagents and conditions: i) NH2-PEGn linker with n=2, 3, 4, 5, 6, or 7, TBTU, DIPEA in DMF, rt, 67-95%; ii) TFA 20% (v/v) in DCM, rt; iii) AP1867 ligand, TBTU, DIPEA in DMF, rt, 39-69% over two steps.

### Synthesis of (+)-JQ1-Based CEMs, CEM207 (19)-CEM212 (24)

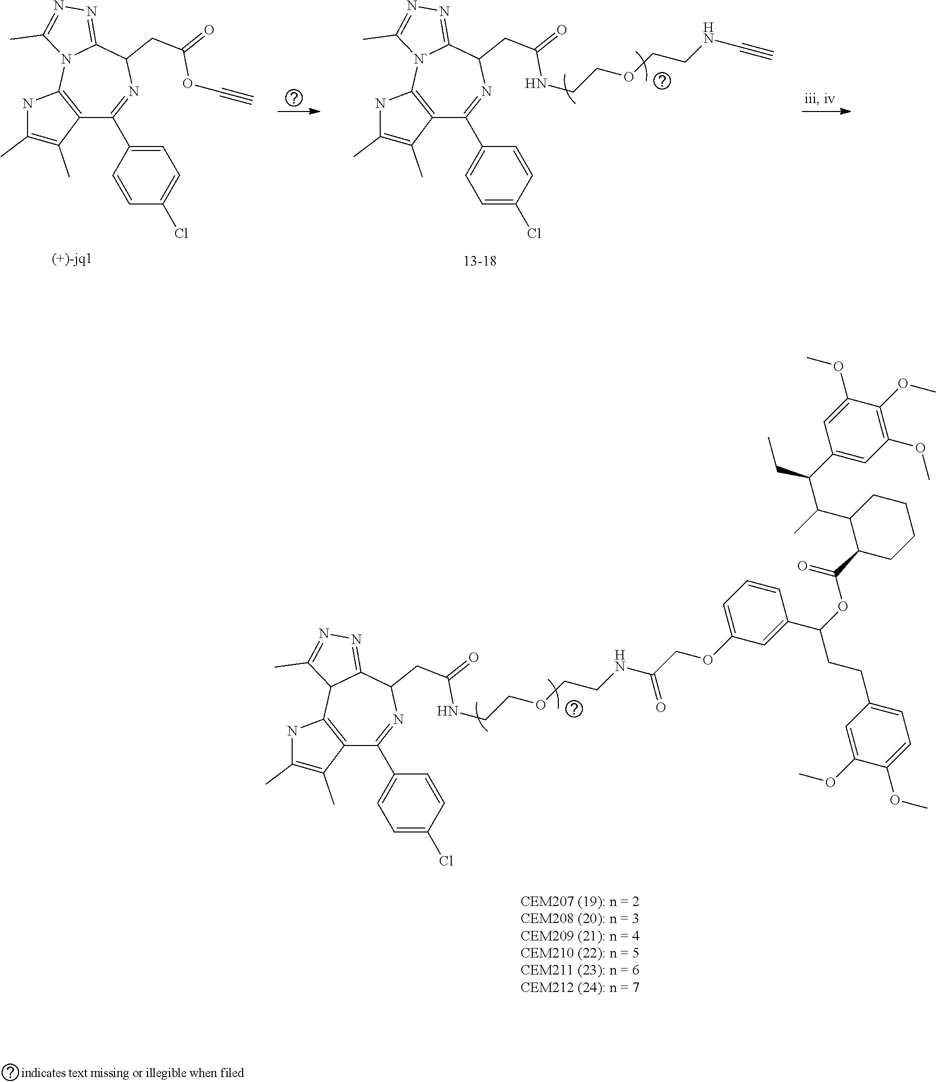

Supplemental scheme 2. Reagents and conditions: i) TFA 20% (v/v) in DCM, rt; ii) NH2-PEGn linker with n=2, 3, 4, 5, 6, or 7, TBTU, DIPEA in DMF, rt, 52-99% over two steps; iii) TFA 20% (v/v) in DCM, rt; iv) AP1867 ligand, TBTU, DIPEA in DMF, rt, 21-63% over two steps.

### Synthetic Procedures

General Procedure 1 (Scheme S1, Step i): Amidation of I-BET762-COOH with NH2-PEGn Linkers

To a solution of I-BET762-COOH (1.0 equiv.) in DMF (0.025 molar) was added TBTU (1.2 equiv.) and DIPEA (1.5 equiv.). After 5-10 min, a solution of NH2-PEGn-CH2CH2NH-Boc (1.1 equiv.) with n=2, 3, 4, 5, 6, or 7 in DMF (0.025 molar) was added to the initial reaction mixture, and the reaction was left to stir at rt. After 1 h, the reaction was concentrated under reduced pressure and purified by automated flash-column chromatography (0-20%, MeOH in DCM) to yield the desired product, compound 1, 2, 3, 4, 5, or 6, as a clear colorless oil.

General Procedure 2 (Scheme S1, Steps ii-iii): Amidation of Compounds 1-6 with AP1867

Compounds 1-6 were N-Boc deprotected in 20% (v/v) TFA in DCM (2.0 mL) at rt and concentrated under reduced pressure. Each deprotected intermediate (1.0 equiv.) was then re-dissolved in DMF (0.02 molar) and DIPEA (1.5 equiv.) to neutralize any remaining excess TFA.

In a separate flask, AP1867 (1.0 equiv.) was dissolved in DMF (0.02 molar) and pre-activated with TBTU (1.2 equiv.) and DIPEA (1.5 equiv.); after 5-10 min, the solution was added to the initial reaction mixture containing the deprotected amine intermediate, and the reaction was left to stir at rt. After 1-24 h, the reaction was concentrated under reduced pressure and purified by preparative HPLC (10-100%, MeOH in water containing 0.1% TFA) to yield the desired product, compound 7, 8, 9, 10, 11, or 12, as a white solid following lyophilization.

General Procedure 3 (Scheme S2, Steps i-ii): Amidation of (+)-JQ1-COOH with NH2-PEGn Linkers

(+)-JQ1 (1.0 equiv.) was hydrolyzed in 20% (v/v) TFA in DCM (2.0 mL) at rt and concentrated under reduced pressure. The intermediate, (+)-JQ1-COOH, was then re-dissolved in DMF (0.025 molar) and pre-activated with TBTU (1.2 equiv.) and DIPEA (3.0 equiv.). After 5-10 min, a solution of NH2-PEGn-CH2CH2NH-Boc (1.1 equiv.) with n=2, 3, 4, 5, 6, or 7 in DMF (0.025 molar) was added to the initial reaction mixture, and the reaction was left to stir at rt. After 1 h, the reaction was concentrated under reduced pressure and purified by automated flash-column chromatography (0-10%, MeOH in DCM) to yield the desired product, compound 13, 14, 15, 16, 17, or 18, as a clear colorless oil.

General Procedure 4 (Scheme S2, Steps iii-iv): Amidation of Compounds 13-18 with AP1867

Compounds 13-18 were N-Boc deprotected in 20% (v/v) TFA in DCM (2.0 mL) at rt and concentrated under reduced pressure. Each deprotected intermediate (1.0 equiv.) was then re-dissolved in DMF (0.02 molar) and DIPEA (1.5 equiv.) to neutralize any remaining excess TFA. In a separate flask, AP1867 (1.0 equiv.) was dissolved in DMF (0.02 molar) and pre-activated with TBTU (1.2 equiv.) and DIPEA (1.5 equiv.); after 5-10 min, the solution was added to the initial reaction mixture containing the deprotected amine intermediate, and the reaction was left to stir at rt. After 1-24 h, the reaction was concentrated under reduced pressure and purified by preparative HPLC (10-100%, MeOH in water containing 0.1% TFA) to yield the desired product, compound 19, 20, 21, 22, 23, or 24, as a white solid following lyophilization.

### tert-Butyl (2-(2-(2-(2-((4S)-6-(4-chlorophenyl)-8-methoxy-1-methyl-4H-benzo[f][1,2,4]triazolo[4,3-a][1,4]diazepin-4-yl) acetamido) ethoxy) ethoxy)ethyl) carbamate (1)

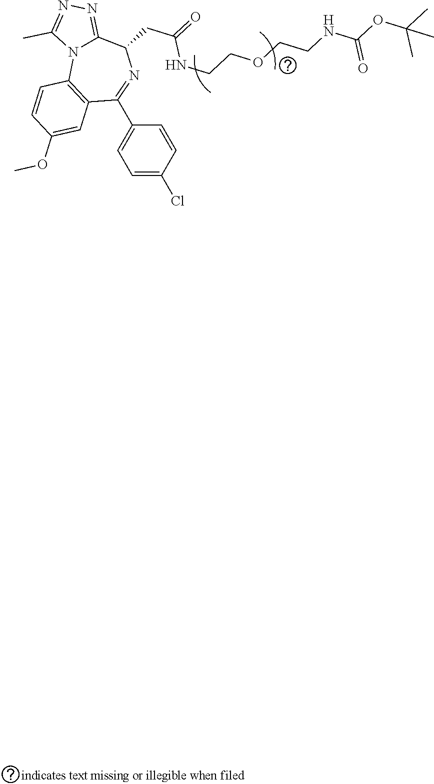

Compound 1 was synthesized from I-BET762-COOH (10.0 mg, 0.025 mmol) and tert-butyl (2-(2-(2-aminoethoxy) ethoxy)ethyl) carbamate according to General Procedure 1. Obtained 10.6 mg as a clear colorless oil, 67%. ESI-MS (m/z): [M+H]+ calcd. for C31H40ClN6O6+, 627.26; found 627.25.

1H NMR (400 MHZ, CD3OD-d4) δ 7.72 (d, J=9.0 Hz, 1H), 7.56 (d, J=8.6 Hz, 2H), 7.41 (d, J=8.6 Hz, 2H), 7.37 (dd, J=9.0, 2.9 Hz, 1H), 6.92 (d, J=2.9 Hz, 1H), 4.63 (dd, J=9.0, 5.2 Hz, 1H), 3.82 (s, 3H), 3.66-3.59 (m, 6H), 3.51 (t, J=5.6 Hz, 2H), 3.47-3.40 (m, 3H), 3.27 (dd, J=14.9, 5.2 Hz, 1H), 3.22 (t, J=5.6 Hz, 2H), 2.63 (s, 3H), 1.41 (s, 9H). 13C NMR (101 MHz, CD3OD-d4) δ 173.02, 172.93, 168.60, 159.90, 157.77, 152.94, 138.62, 138.10, 132.25, 131.33, 129.52, 127.40, 126.75, 119.17, 117.02, 71.35, 71.32, 71.10, 70.67, 56.43, 54.59, 41.25, 40.62, 40.50, 38.76, 28.75, 11.68.

### tert-Butyl (1-((4S)-6-(4-chlorophenyl)-8-methoxy-1-methyl-4H-benzo[f][1,2,4]triazolo[4,3-a][1,4]diazepin-4-yl)-2-oxo-6,9,12-trioxa-3-azatetradecan-14-yl)carbamate (2)

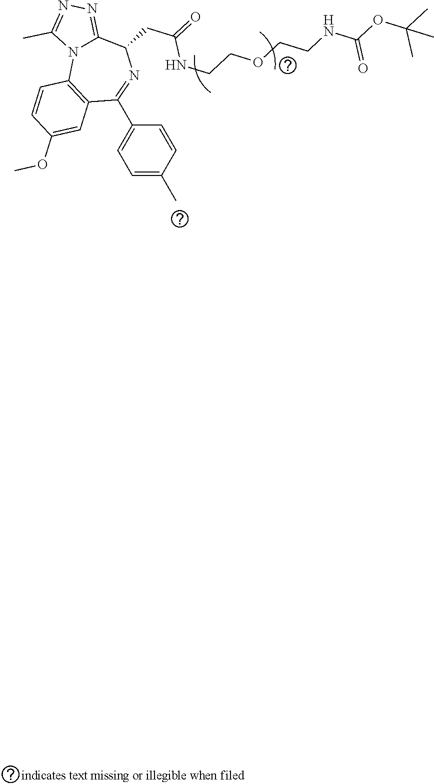

Compound 2 was synthesized from I-BET762-COOH (10.0 mg, 0.025 mmol) and tert-butyl (2-(2-(2-(2-aminoethoxy) ethoxy) ethoxy)ethyl) carbamate according to General Procedure 1. Obtained 16.0 mg as a clear colorless oil, 95%.

ESI-MS (m/z): [M+H]+ calcd. for C33H44ClN6O7+, 671.29; found 671.20.

1H NMR (400 MHZ, CD3OD-d4) δ 7.71 (d, J=8.9 Hz, 1H), 7.56 (d, J=8.6 Hz, 2H), 7.41 (d, J=8.6 Hz, 2H), 7.37 (dd, J=9.0, 2.9 Hz, 1H), 6.91 (d, J=2.9 Hz, 1H), 4.62 (dd, J=9.1, 5.1 Hz, 1H), 3.82 (s, 3H), 3.66-3.58 (m, 10H), 3.49 (t, J=5.6 Hz, 2H), 3.47-3.40 (m, 3H), 3.26 (dd, J=15.0, 5.1 Hz, 1H), 3.20 (t, J=5.6 Hz, 2H), 2.63 (s, 3H), 1.41 (s, 9H).

13C NMR (101 MHZ, CD3OD-d4) δ 172.99, 172.91, 168.54, 159.87, 157.76, 152.93, 138.61, 138.07, 132.24, 131.31, 129.52, 127.39, 126.74, 119.15, 117.02, 71.58, 71.56, 71.30, 71.23, 71.06, 70.62, 56.43, 54.59, 41.26, 40.64, 40.52, 38.77, 28.76, 11.69.

### tert-Butyl (1-((4S)-6-(4-chlorophenyl)-8-methoxy-1-methyl-4H-benzo[f][1,2,4]triazolo[4,3-a][1,4]diazepin-4-yl)-2-oxo-6,9,12,15-tetraoxa-3-azaheptadecan-17-yl)carbamate (3)

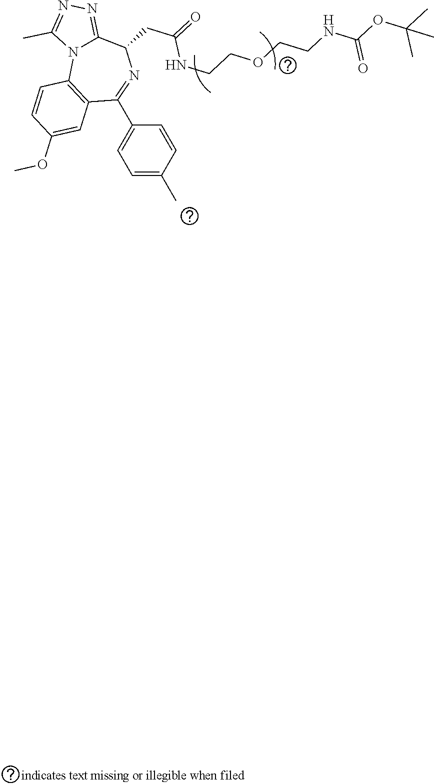

Compound 3 was synthesized from I-BET762-COOH (10.0 mg, 0.025 mmol) and tert-butyl (14-amino-3,6,9,12-tetraoxatetradecyl) carbamate according to General Procedure 1. Obtained 16.0 mg as a clear colorless oil, 89%.

ESI-MS (m/z): [M+H]+ calcd. for C35H48ClN6O8+, 715.31; found 715.25.

1H NMR (400 MHZ, CD3OD-d4) δ 7.72 (d, J=8.9 Hz, 1H), 7.56 (d, J=8.6 Hz, 2H), 7.41 (d, J=8.6 Hz, 2H), 7.37 (dd, J=9.0, 2.9 Hz, 1H), 6.91 (d, J=2.9 Hz, 1H), 4.62 (dd, J=9.2, 5.0 Hz, 1H), 3.82 (s, 3H), 3.66-3.56 (m, 14H), 3.48 (t, J=5.6 Hz, 3H), 3.46-3.39 (m, 2H), 3.26 (dd, J=15.0, 5.1 Hz, 1H), 3.19 (t, J=5.6 Hz, 2H), 2.63 (s, 3H), 1.42 (s, 9H).

13C NMR (101 MHZ, CD3OD-d4) δ 172.93, 168.54, 159.88, 157.78, 152.94, 138.63, 138.07, 132.25, 131.32, 129.53, 127.40, 126.75, 119.16, 117.01, 71.59, 71.55, 71.53, 71.35, 71.24, 71.05, 70.64, 56.43, 54.59, 41.27, 40.55, 38.77, 28.77, 11.69. (1 coincident carbonyl peak; 2 coincident aliphatic peaks).

### tert-Butyl (1-((4S)-6-(4-chlorophenyl)-8-methoxy-1-methyl-4H-benzo[f][1,2,4]triazolo[4,3-a][1,4]diazepin-4-yl)-2-oxo-6,9,12,15,18-pentaoxa-3-azaicosan-20-yl) carbamate (4)

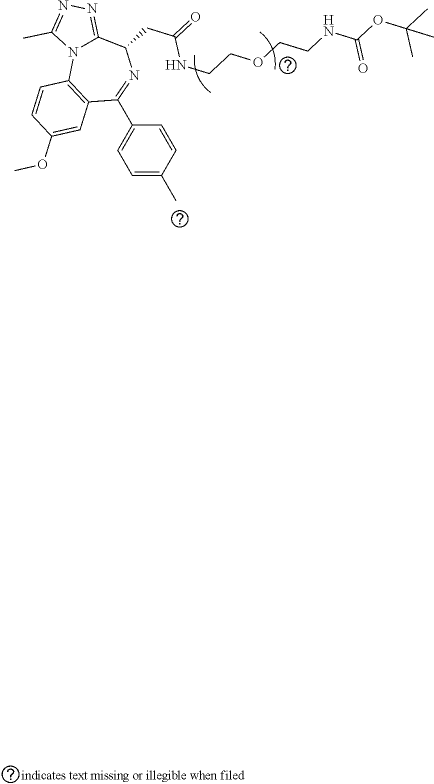

Compound 4 was synthesized from I-BET762-COOH (6.95 mg, 0.018 mmol) and tert-butyl (17-amino-3,6,9,12,15-pentaoxaheptadecyl) carbamate according to General Procedure 1. Obtained 6.6 mg as a clear colorless oil, 50%.

ESI-MS (m/z): [M+H]+ calcd. for C37H52ClN6O9+, 759.34; found 759.30.

1H NMR (400 MHZ, CD3OD-d4) δ 7.71 (d, J=9.0 Hz, 1H), 7.55 (d, J=8.6 Hz, 2H), 7.41 (d, J=8.6 Hz, 2H), 7.37 (dd, J=9.0, 2.9 Hz, 1H), 6.91 (d, J=2.9 Hz, 1H), 4.62 (dd, J=9.2, 5.0 Hz, 1H), 3.82 (s, 3H), 3.65-3.54 (m, 18H), 3.47 (t, J=5.6 Hz, 2H), 3.45-3.36 (m, 3H), 3.25 (dd, J=14.9, 5.0 Hz, 1H), 3.19 (t, J=5.6 Hz, 2H), 2.63 (s, 3H), 1.42 (s, 9H).

13C NMR (101 MHz, CD3OD-d4) δ 172.94, 168.55, 159.91, 157.79, 152.95, 138.64, 138.09, 132.26, 131.34, 129.54, 127.41, 126.76, 119.18, 117.01, 71.61, 71.60, 71.55, 71.53, 71.53, 71.36, 71.25, 71.05, 70.64, 56.43, 54.60, 41.29, 40.57, 38.78, 28.77, 11.68. (1 coincident carbonyl peak; 2 coincident aliphatic peaks).

### tert-Butyl (1-((4S)-6-(4-chlorophenyl)-8-methoxy-1-methyl-4H-benzo[f][1,2,4]triazolo[4,3-a][1,4]diazepin-4-yl)-2-oxo-6,9,12,15,18,21-hexaoxa-3-azatricosan-23-yl) carbamate (5)

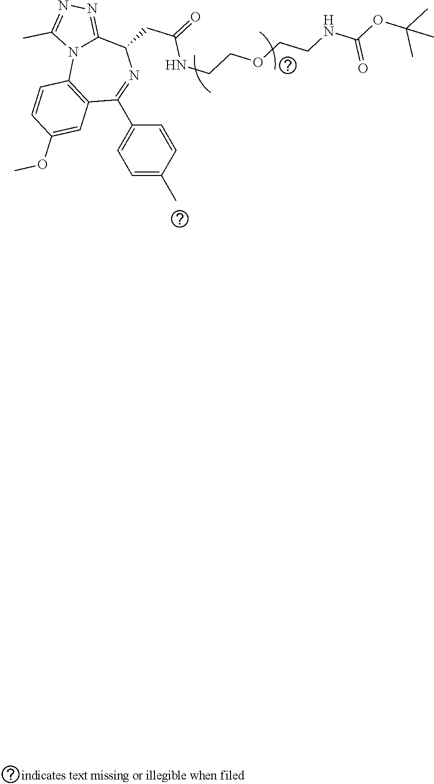

Compound 5 was synthesized from I-BET762-COOH (10.0 mg, 0.025 mmol) and tert-butyl (20-amino-3,6,9,12,15,18-hexaoxaicosyl) carbamate according to General Procedure 1. Obtained 14.2 mg as a clear colorless oil, 70%.

ESI-MS (m/z): [M+H]+ calcd. for C39H56ClN6O10+, 803.37; found 803.30.

1H NMR (400 MHZ, CD3OD-d4) δ 7.72 (d, J=8.9 Hz, 1H), 7.56 (d, J=8.6 Hz, 2H), 7.42 (d, J=8.6 Hz, 2H), 7.38 (dd, J=9.0, 2.9 Hz, 1H), 6.92 (d, J=2.9 Hz, 1H), 4.62 (dd, J=9.2, 5.0 Hz, 1H), 3.82 (s, 3H), 3.65-3.56 (m, 22H), 3.48 (t, J=5.6 Hz, 2H), 3.47-3.38 (m, 3H), 3.26 (dd, J=15.0, 5.0 Hz, 1H), 3.20 (t, J=5.6 Hz, 2H), 2.64 (s, 3H), 1.42 (s, 9H).

13C NMR (101 MHZ, CD3OD-d4) δ 173.00, 172.92, 168.52, 159.88, 157.78, 152.94, 138.64, 138.07, 132.26, 131.33, 129.54, 127.40, 126.75, 119.16, 117.01, 71.61, 71.60, 71.56, 71.55, 71.52, 71.36, 71.25, 71.04, 70.63, 56.44, 54.60, 41.29, 40.69, 40.57, 38.78, 28.78, 11.69. (3 coincident aliphatic peaks)

### tert-Butyl (1-((4S)-6-(4-chlorophenyl)-8-methoxy-1-methyl-4H-benzo[f][1,2,4]triazolo[4,3-a][1,4]diazepin-4-yl)-2-oxo-6,9,12,15,18,21,24-heptaoxa-3-azahexacosan-26-yl) carbamate (6)

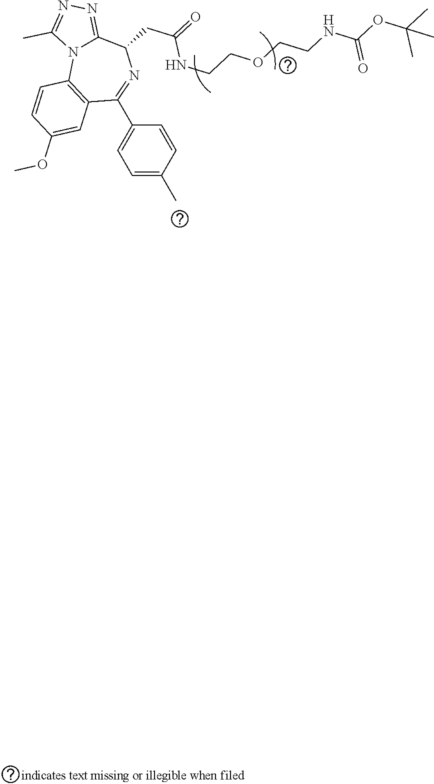

Compound 6 was synthesized from I-BET762-COOH (6.3 mg, 0.016 mmol) and tert-butyl (23-amino-3,6,9,12,15,18,21-heptaoxatricosyl) carbamate according to General Procedure 1. Obtained 9.2 mg as a clear colorless oil, 68%.

ESI-MS (m/z): [M+H]+ calcd. for C41H60ClN6O11+, 847.39; found 847.30.

1H NMR (400 MHZ, CD3OD-d4) δ 7.72 (d, J=9.0 Hz, 1H), 7.56 (d, J=8.6 Hz, 2H), 7.42 (d, J=8.6 Hz, 2H), 7.38 (dd, J=9.0, 2.9 Hz, 1H), 6.92 (d, J=2.9 Hz, 1H), 4.62 (dd, J=9.2, 5.0 Hz, 1H), 3.83 (s, 3H), 3.66-3.57 (m, 26H), 3.49 (t, J=5.6 Hz, 2H), 3.47-3.38 (m, 3H), 3.26 (dd, J=15.0, 5.1 Hz, 1H), 3.20 (t, J=5.6 Hz, 2H), 2.64 (s, 3H), 1.43 (s, 9H).

13C NMR (101 MHz, CD3OD-d4) δ 172.94, 168.54, 159.89, 157.78, 152.94, 138.64, 138.08, 132.27, 131.33, 129.55, 127.41, 126.76, 119.17, 117.02, 71.59, 71.57, 71.53, 71.52, 71.51, 71.49, 71.34, 71.24, 71.07, 70.65, 56.44, 54.60, 41.29, 40.56, 38.78, 28.78, 11.69. (1 coincident carbonyl peak; 5 coincident aliphatic peaks).

### (1R)-1-(3-((14-((4S)-6-(4-Chlorophenyl)-8-methoxy-1-methyl-4H-benzo[f][1,2,4]triazolo[4,3-a][1,4]diazepin-4-yl)-2,13-dioxo-6,9-dioxa-3,12-diazatetradecyl)oxy)phenyl)-3-(3,4-dimethoxyphenyl) propyl (2S)-1-((S)-2-(3,4,5-trimethoxyphenyl) butanoyl) piperidine-2-carboxylate (CEM201, 7)

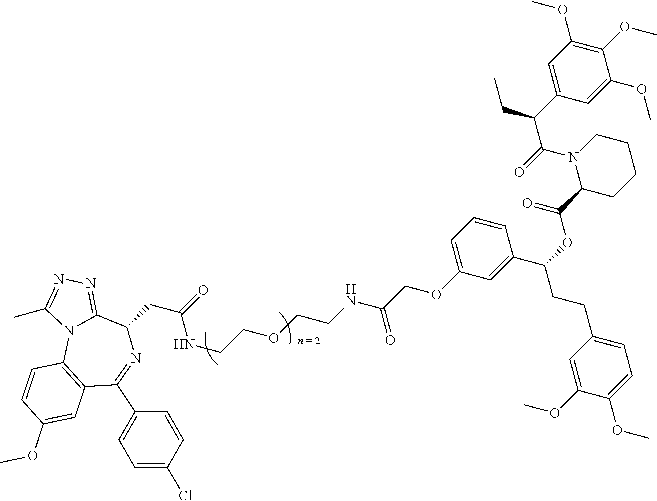

CEM201 (7) was synthesized from compound 1 (5.3 mg, 0.008 mmol) according to General Procedure 2.

Obtained 7.0 mg as a white solid, 69% over two steps.

ESI-MS (m/z): [M+H]+ calcd. for C64H77ClN7O14+, 1202.51; found 1202.40.

1H NMR (400 MHZ, CD3OD-d4) δ 7.72 (d, J=9.0 Hz, 1H), 7.55 (d, J=8.6 Hz, 2H), 7.38 (d, J=8.7 Hz, 3H), 7.15 (t, J=8.0 Hz, 1H), 6.92 (d, J=2.9 Hz, 1H), 6.86-6.81 (m, 2H), 6.79-6.63 (m, 3H), 6.63-6.51 (m, 3H), 5.56 (dd, J=8.3, 5.5 Hz, 1H), 5.37 (d, J=4.3 Hz, 1H), 4.67 (dd, J=8.7, 5.3 Hz, 1H), 4.53-4.45 (m, 2H), 4.08 (d, J=13.3 Hz, 1H), 3.89-3.83 (m, 1H), 3.82-3.80 (m, 4H), 3.80-3.76 (m, 6H), 3.74 (s, 1H), 3.69-3.65 (m, 7H), 3.62-3.55 (m, 8H), 3.49-3.39 (m, 5H), 3.29-3.24 (m, 1H), 2.76-2.66 (m, 4H), 2.57-2.47 (m, 1H), 2.46-2.36 (m, 1H), 2.27 (m, 1H), 2.06-1.95 (m, 2H), 1.92-1.81 (m, 1H), 1.74-1.56 (m, 4H), 1.54-1.43 (m, 1H), 1.30-1.19 (m, 1H), 0.89 (t, J=7.3 Hz, 3H).

13C NMR (101 MHZ, CD3OD-d4) δ 172.56, 171.85, 171.07, 168.74, 160.36, 159.07, 154.55, 150.40, 148.83, 143.59, 138.37, 138.23, 137.91, 136.92, 135.12, 132.32, 131.52, 130.88, 129.52, 126.91, 126.46, 121.75, 121.67, 120.63, 119.19, 117.31, 115.05, 114.43, 113.64, 113.23, 106.56, 105.98, 77.12, 71.37, 71.34, 70.65, 70.48, 68.29, 61.08, 56.74, 56.55, 56.50, 56.45, 54.30, 53.61, 51.22, 44.97, 40.51, 39.96, 39.22, 38.54, 32.25, 29.38, 27.61, 26.31, 21.87, 12.67, 11.48.

### (1R)-1-(3-((17-((4S)-6-(4-Chlorophenyl)-8-methoxy-1-methyl-4/I-benzo[f][1,2,4]triazolo[4,3-a][1,4]diazepin-4-yl)-2,16-dioxo-6,9,12-trioxa-3,15-diazaheptadecyl)oxy)phenyl)-3-(3,4-dimethoxyphenyl) propyl (2S)-1-((S)-2-(3,4,5-trimethoxyphenyl) butanoyl) piperidine-2-carboxylate (CEM202, 8)

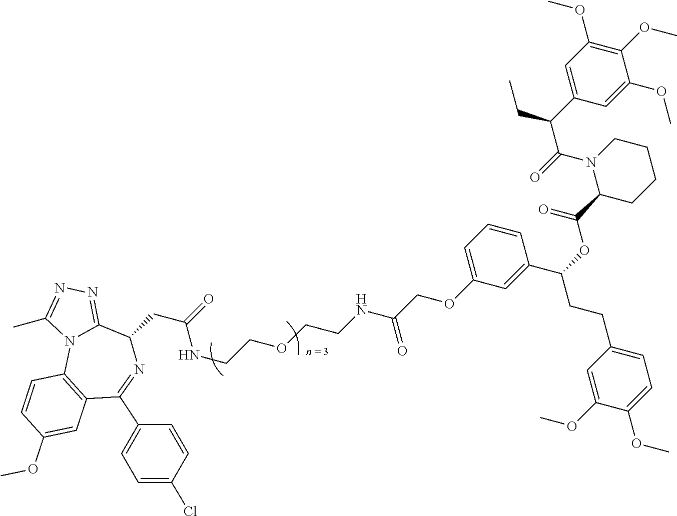

CEM202 (8) was synthesized from compound 2 (5.3 mg, 0.008 mmol) according to General Procedure 2.

Obtained 4.3 mg as a white solid, 44% over two steps.

ESI-MS (m/z): [M+H]+ calcd. for C66H81ClN7O15+, 1246.54; found 1246.40.

1H NMR (400 MHZ, CD3OD-d4) δ 7.72 (d, J=9.0 Hz, 1H), 7.55 (d, J=8.6 Hz, 2H), 7.42-7.36 (m, 3H), 7.16 (t, J=7.9 Hz, 1H), 6.93 (d, J=2.9 Hz, 1H), 6.85-6.81 (m, 2H), 6.79-6.64 (m, 3H), 6.62-6.52 (m, 3H), 5.57 (dd, J=8.3, 5.4 Hz, 1H), 5.38 (d, J=5.6 Hz, 1H), 4.66 (dd, J=8.8, 5.3 Hz, 1H), 4.53-4.44 (m, 2H), 4.08 (d, J=13.4 Hz, 1H), 3.89-3.84 (m, 1H), 3.82-3.80 (m, 4H), 3.80-3.77 (m, 6H), 3.74 (s, 1H), 3.69-3.66 (m, 7H), 3.63-3.53 (m, 12H), 3.47-3.39 (m, 5H), 3.26 (dd, J=15.1, 5.4 Hz, 1H), 2.77-2.67 (m, 4H), 2.57-2.48 (m, 1H), 2.47-2.36 (m, 1H), 2.28 (m, 1H), 2.01 (m, 2H), 1.92-1.82 (m, 1H), 1.76-1.57 (m, 4H), 1.55-1.45 (m, 1H), 1.28-1.17 (m, 1H), 0.89 (t, J=7.3 Hz, 3H).

13C NMR (101 MHz, CD3OD-d4) δ 174.96, 172.62, 171.85, 171.02, 168.70, 160.27, 159.08, 154.57, 150.41, 148.84, 143.60, 138.43, 138.19, 137.93, 136.92, 135.12, 132.32, 131.50, 130.90, 129.53, 126.88, 126.66, 121.76, 121.68, 120.64, 119.19, 117.24, 115.09, 114.45, 113.65, 113.23, 106.57, 105.98, 77.11, 71.62, 71.58, 71.32, 71.28, 70.60, 70.44, 68.29, 61.08, 56.74, 56.55, 56.49, 56.45, 54.36, 53.61, 51.22, 44.98, 40.54, 40.00, 39.24, 38.59, 32.25, 29.39, 27.62, 26.32, 21.87, 12.67, 11.53.

### (1R)-1-(3-((20-((4S)-6-(4-Chlorophenyl)-8-methoxy-1-methyl-4H-benzo[f][1,2,4]triazolo[4,3-a][1,4]diazepin-4-yl)-2,19-dioxo-6,9,12,15-tetraoxa-3,18-diazaicosyl)oxy)phenyl)-3-(3,4-dimethoxyphenyl) propyl (2S)-1-((S)-2-(3,4,5-trimethoxyphenyl) butanoyl) piperidine-2-carboxylate (CEM203, 9)

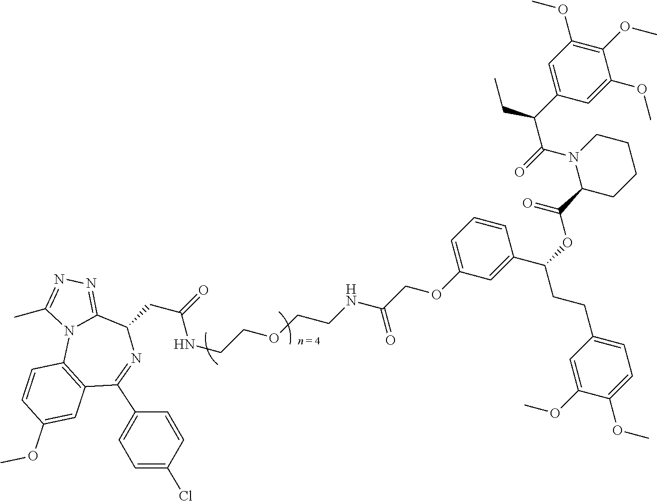

CEM203 (9) was synthesized from compound 3 (8.0 mg, 0.011 mmol) according to General Procedure 2.

Obtained 5.6 mg as a white solid, 39% over two steps.

ESI-MS (m/z): [M+H]+ calcd. for C68H87ClN7O16+, 1290.57; found 1290.40.

1H NMR (400 MHZ, CD3OD-d4) δ 7.73 (d, J=9.0 Hz, 1H), 7.56 (d, J=8.6 Hz, 2H), 7.42-7.36 (m, 3H), 7.17 (t, J=7.9 Hz, 1H), 6.94 (d, J=2.9 Hz, 1H), 6.87-6.82 (m, 2H), 6.80-6.65 (m, 3H), 6.62-6.53 (m, 3H), 5.57 (dd, J=8.3, 5.5 Hz, 1H), 5.38 (d, J=5.4 Hz, 1H), 4.67 (dd, J=8.9, 5.3 Hz, 1H), 4.53-4.45 (m, 2H), 4.08 (d, J=13.2 Hz, 1H), 3.89-3.84 (m, 1H), 3.83-3.81 (m, 4H), 3.80-3.77 (m, 6H), 3.74 (s, 1H), 3.69-3.65 (m, 7H), 3.61-3.52 (m, 16H), 3.48-3.40 (m, 5H), 3.29-3.24 (m, 1H), 2.76-2.68 (m, 4H), 2.56-2.49 (m, 1H), 2.47-2.38 (m, 1H), 2.28 (m, 1H), 2.06-1.98 (m, 2H), 1.93-1.83 (m, 1H), 1.75-1.57 (m, 4H), 1.55-1.46 (m, 1H), 1.27-1.18 (m, 1H), 0.89 (t, J=7.3 Hz, 3H).

13C NMR (101 MHz, CD3OD-d4) δ 174.96, 172.61, 171.85, 171.01, 168.71, 160.34, 159.09, 154.57, 150.41, 148.85, 143.62, 138.42, 138.21, 137.93, 136.92, 135.12, 132.33, 131.53, 130.90, 129.54, 126.90, 126.56, 121.76, 121.69, 120.64, 119.20, 117.27, 115.11, 114.43, 113.65, 113.24, 106.57, 105.99, 77.11, 71.59, 71.57, 71.54, 71.32, 71.30, 70.62, 70.43, 68.28, 61.08, 56.75, 56.55, 56.51, 56.46, 54.34, 53.61, 51.22, 44.98, 40.56, 40.01, 39.25, 38.57, 32.26, 29.39, 27.62, 26.32, 21.87, 12.68, 11.51. (1 coincident aliphatic peak).

### (1R)-1-(3-((23-((4S)-6-(4-Chlorophenyl)-8-methoxy-1-methyl-4H-benzo[f][1,2,4]triazolo[4,3-a][1,4]diazepin-4-yl)-2,22-dioxo-6,9,12,15,18-pentaoxa-3,21-diazatricosyl)oxy)phenyl)-3-(3,4-dimethoxyphenyl) propyl (2S)-1-((S)-2-(3,4,5-trimethoxyphenyl) butanoyl) piperidine-2-carboxylate (CEM204, 10)

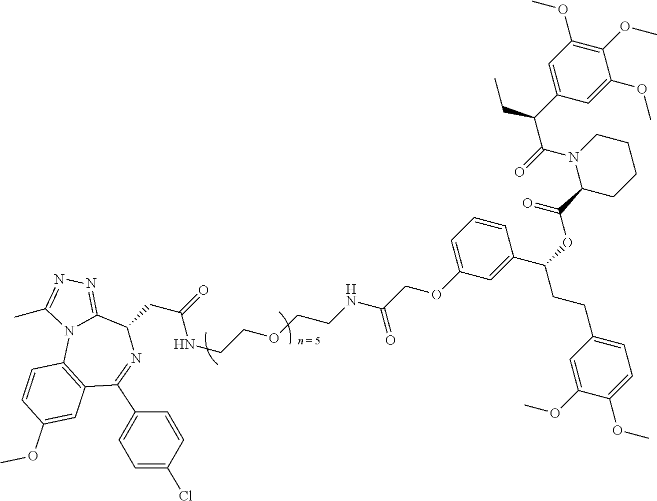

CEM204 (10) was synthesized from compound 4 (4.9 mg, 0.006 mmol) according to General Procedure 2.

Obtained 5.6 mg as a white solid, 66% over two steps.

ESI-MS (m/z): [M+H]+ calcd. for C70H89ClN7O17+, 1334.59; found 1334.40.

1H NMR (400 MHZ, CD3OD-d4) δ 7.70 (d, J=9.0 Hz, 1H), 7.55 (d, J=8.6 Hz, 2H), 7.42-7.34 (m, 3H), 7.17 (t, J=7.9 Hz, 1H), 6.91 (d, J=2.8 Hz, 1H), 6.87-6.82 (m, 2H), 6.79-6.65 (m, 3H), 6.62-6.53 (m, 3H), 5.57 (dd, J=8.4, 5.3 Hz, 1H), 5.38 (d, J=5.6 Hz, 1H), 4.63 (dd, J=9.0, 5.1 Hz, 1H), 4.54-4.45 (m, 2H), 4.08 (d, J=13.6 Hz, 1H), 3.89-3.84 (m, 1H), 3.82-3.80 (m, 4H), 3.80-3.77 (m, 6H), 3.74 (s, 1H), 3.69-3.66 (m, 7H), 3.63-3.51 (m, 20H), 3.48-3.39 (m, 5H), 3.29-3.22 (m, 1H), 2.72 (td, J=13.6, 3.1 Hz, 1H), 2.64 (s, 3H), 2.56-2.47 (m, 1H), 2.46-2.37 (m, 1H), 2.28 (m, 1H), 2.06-1.97 (m, 2H), 1.92-1.82 (m, 1H), 1.74-1.59 (m, 4H), 1.55-1.47 (m, 1H), 1.26-1.20 (m, 1H), 0.89 (t, J=7.3 Hz, 3H).

13C NMR (126 MHz, CD3OD-d4) δ 174.96, 172.94, 171.86, 171.11, 168.56, 159.95, 159.10, 154.57, 150.41, 148.84, 143.61, 138.59, 138.09, 137.93, 136.92, 135.12, 132.26, 131.35, 130.91, 129.54, 127.28, 126.78, 121.77, 121.69, 120.64, 119.16, 117.07, 115.13, 114.44, 113.65, 113.24, 106.57, 105.99, 77.11, 71.56, 71.54, 71.51, 71.50, 71.32, 71.27, 70.67, 70.44, 68.29, 61.09, 56.75, 56.56, 56.45, 56.45, 54.57, 53.60, 51.22, 44.98, 40.56, 40.00, 39.25, 38.76, 32.25, 29.38, 27.61, 26.32, 21.87, 12.67, 11.67. (2 coincident aliphatic peaks).

### (1R)-1-(3-((26-((4S)-6-(4-Chlorophenyl)-8-methoxy-1-methyl-4H-benzo[f][1,2,4]triazolo[4,3-a][1,4]diazepin-4-yl)-2,25-dioxo-6,9,12,15,18,21-hexaoxa-3,24-diazahexacosyl)oxy)phenyl)-3-(3,4-dimethoxyphenyl) propyl (2S)-1-((S)-2-(3,4,5-trimethoxyphenyl) butanoyl) piperidine-2-carboxylate (CEM205, 11)

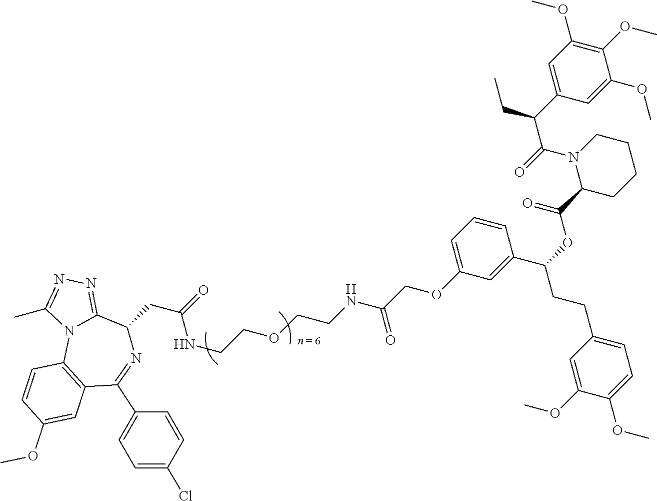

CEM205 (11) was synthesized from compound 5 (9.1 mg, 0.011 mmol) according to General Procedure 2.

Obtained 10.2 mg as a white solid, 65% over two steps.

ESI-MS (m/z): [M+H]+ calcd. for C72H93ClN7O18+, 1378.62; found 1378.40.

1H NMR (400 MHZ, CD3OD-d4) δ 7.74 (d, J=9.0 Hz, 1H), 7.57 (d, J=8.6 Hz, 2H), 7.43-7.37 (m, 3H), 7.18 (t, J=7.9 Hz, 1H), 6.95 (d, J=2.8 Hz, 1H), 6.87-6.82 (m, 2H), 6.81-6.64 (m, 3H), 6.62-6.53 (m, 3H), 5.58 (dd, J=8.2, 5.5 Hz, 1H), 5.38 (d, J=4.5 Hz, 1H), 4.69 (dd, J=8.8, 5.3 Hz, 1H), 4.54-4.48 (m, 2H), 4.08 (d, J=13.6 Hz, 1H), 3.89-3.84 (m, 1H), 3.83-3.81 (m, 4H), 3.80-3.77 (m, 6H), 3.74 (s, 1H), 3.70-3.66 (m, 7H), 3.63-3.53 (m, 24H), 3.49-3.40 (m, 5H), 3.30-3.25 (m, 1H), 2.77-2.67 (m, 4H), 2.57-2.49 (m, 1H), 2.47-2.38 (m, 1H), 2.28 (m, 1H), 2.06-1.96 (m, 2H), 1.93-1.83 (m, 1H), 1.76-1.57 (m, 4H), 1.56-1.43 (m, 1H), 1.28-1.17 (m, 1H), 0.89 (t, J=7.3 Hz, 3H).

13C NMR (101 MHZ, CD3OD-d4) δ 174.95, 172.54, 171.85, 171.01, 168.75, 160.45, 159.10, 154.56, 150.41, 148.85, 143.62, 138.37, 138.25, 137.92, 136.92, 135.11, 132.36, 131.59, 130.91, 129.54, 126.95, 126.35, 121.76, 121.69, 120.63, 119.22, 117.34, 115.13, 114.42, 113.65, 113.24, 106.56, 105.98, 77.11, 71.59, 71.54, 71.52, 71.51, 71.32, 71.29, 70.61, 70.41, 68.28, 61.08, 56.75, 56.55, 56.53, 56.46, 54.28, 53.61, 51.22, 44.97, 40.57, 40.02, 39.27, 38.51, 32.26, 29.39, 27.62, 26.32, 21.88, 12.69, 11.47. (4 coincident aliphatic peaks).

### (1R)-1-(3-((29-((4S)-6-(4-Chlorophenyl)-8-methoxy-1-methyl-4H-benzo[f][1,2,4]triazolo[4,3-a][1,4]diazepin-4-yl)-2,28-dioxo-6,9,12,15,18,21,24-heptaoxa-3,27-diazanonacosyl)oxy)phenyl)-3-(3,4-dimethoxyphenyl) propyl (2S)-1-((S)-2-(3,4,5-trimethoxyphenyl) butanoyl) piperidine-2-carboxylate (CEM206, 12)

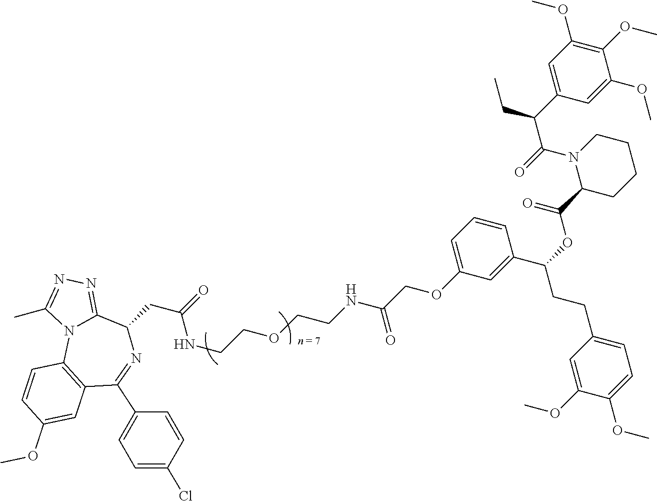

CEM206 (12) was synthesized from compound 6 (4.2 mg, 0.005 mmol) according to General Procedure 2.

Obtained 4.3 mg as a white solid, 60% over two steps.

ESI-MS (m/z): [M+H]+ calcd. for C74H97ClN7O19+, 1422.64; found 1422.50.

1H NMR (400 MHZ, CD3OD-d4) δ 7.71 (d, J=8.9 Hz, 1H), 7.54 (d, J=8.6 Hz, 2H), 7.41-7.35 (m, 3H), 7.16 (t, J=8.0 Hz, 1H), 6.91 (d, J=2.9 Hz, 1H), 6.86-6.82 (m, 2H), 6.80-6.64 (m, 3H), 6.61-6.52 (m, 3H), 5.57 (dd, J=8.3, 5.5 Hz, 1H), 5.37 (d, J=5.3 Hz, 1H), 4.64 (dd, J=9.0, 5.2 Hz, 1H), 4.53-4.44 (m, 2H), 4.07 (d, J=13.6 Hz, 1H), 3.85 (t, J=7.3 Hz, 1H), 3.81-3.79 (m, 4H), 3.79-3.77 (m, 6H), 3.73 (s, 1H), 3.68-3.65 (m, 7H), 3.62-3.53 (m, 28H), 3.47-3.38 (m, 5H), 3.25 (dd, J=15.0, 5.1 Hz, 1H), 2.75-2.63 (m, 4H), 2.57-2.48 (m, 1H), 2.45-2.37 (m, 1H), 2.27 (m, 1H), 2.05-1.96 (m, 2H), 1.92-1.82 (m, 1H), 1.74-1.56 (m, 4H), 1.55-1.47 (m, 1H), 1.26-1.16 (m, 1H), 0.88 (t, J=7.3 Hz, 3H).

13C NMR (126 MHz, CD3OD-d4) δ 174.96, 172.83, 171.86, 171.04, 168.59, 160.03, 159.11, 154.57, 150.41, 148.85, 143.61, 138.56, 138.12, 137.94, 136.92, 135.12, 132.29, 131.39, 130.91, 129.54, 127.13, 126.81, 121.77, 121.69, 120.64, 119.18, 117.11, 115.14, 114.44, 113.65, 113.24, 106.57, 105.99, 77.11, 71.58, 71.55, 71.51, 71.49, 71.33, 71.28, 70.64, 70.43, 68.30, 61.09, 56.75, 56.56, 56.47, 54.52, 53.61, 51.22, 44.98, 40.56, 40.02, 39.26, 38.72, 32.26, 29.39, 27.62, 26.32, 21.87, 12.68, 11.64. (6 coincident aliphatic peaks).

### tert-Butyl(S)-(2-(2-(2-(2-(4-(4-chlorophenyl)-2,3,9-trimethyl-6H-thieno[3,2-f][1,2,4]triazolo[4,3-a][1,4]diazepin-6-yl) acetamido) ethoxy) ethoxy)ethyl) carbamate (13)

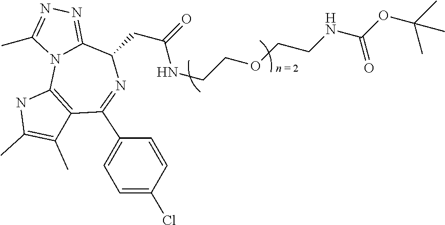

Compound 13 was synthesized from (+)-JQ1 (13.3 mg, 0.029 mmol) and tert-butyl (2-(2-(2-aminoethoxy) ethoxy)ethyl) carbamate according to General Procedure 3. Obtained 13.8 mg as a clear colorless oil, 75% over two steps.

ESI-MS (m/z): [M+H]+ calcd. for C30H40ClN6O5S+, 631.24; found 631.20.

1H NMR (400 MHZ, CD3OD-d4) δ 7.48-7.39 (m, 4H), 4.63 (dd, J=8.8, 5.3 Hz, 1H), 3.66-3.59 (m, 6H), 3.52 (t, J=5.6 Hz, 2H), 3.49-3.41 (m, 3H), 3.35-3.26 (m, 1H) (overlapping CD3OD peak), 3.22 (t, J=5.6 Hz, 2H), 2.69 (s, 3H), 2.45 (s, 3H), 1.70 (s, 3H), 1.41 (s, 9H).

13C NMR (101 MHZ, CD3OD-d4) δ 172.89, 166.16, 157.01, 152.12, 138.10, 137.95, 133.50, 133.20, 132.02, 131.98, 131.35, 129.77, 71.34, 71.31, 71.10, 70.66, 55.18, 41.24, 40.49, 38.74, 28.76, 14.42, 12.93, 11.61. (1 coincident carbonyl peak; 1 coincident aliphatic peak).

### tert-Butyl(S)-(1-(4-(4-chlorophenyl)-2,3,9-trimethyl-6H-thieno[3,2-f][1,2,4]triazolo[4,3-a][1,4]diazepin-6-yl)-2-oxo-6,9,12-trioxa-3-azatetradecan-14-yl) carbamate (14)

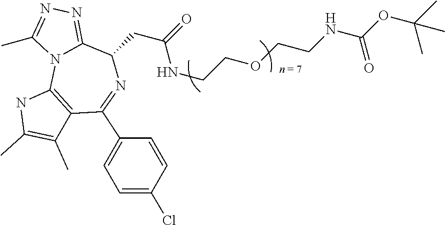

Compound 14 was synthesized from (+)-JQ1 (13.3 mg, 0.029 mmol) and tert-butyl (2-(2-(2-(2-aminoethoxy) ethoxy) ethoxy)ethyl) carbamate according to General Procedure 3. Obtained 10.2 mg as a clear colorless oil, 52% over two steps.

ESI-MS (m/z): [M+H]+ calcd. for C32H44ClN6O6S+, 675.27; found 675.20.

1H NMR (400 MHz, CD3OD-d4) δ 7.48-7.39 (m, 4H), 4.64 (dd, J=8.9, 5.2 Hz, 1H), 3.68-3.59 (m, 10H), 3.50 (t, J=5.6 Hz, 2H), 3.48-3.39 (m, 3H), 3.35-3.26 (m, 1H) (overlapping CD3OD peak), 3.21 (t, J=5.6 Hz, 2H), 2.70 (s, 3H), 2.45 (s, 3H), 1.70 (s, 3H), 1.41 (s, 9H).

13C NMR (101 MHZ, CD3OD-d4) δ 172.87, 166.18, 157.01, 152.17, 138.07, 137.98, 133.49, 133.26, 132.04, 131.99, 131.37, 129.78, 71.59, 71.57, 71.30, 71.24, 71.07, 70.62, 55.16, 41.26, 40.54, 38.70, 28.76, 14.42, 12.93, 11.60. (1 coincident carbonyl peak; 1 coincident aliphatic peak).

### tert-Butyl(S)-(1-(4-(4-chlorophenyl)-2,3,9-trimethyl-6H-thieno[3,2-f][1,2,4]triazolo[4,3-a][1,4]diazepin-6-yl)-2-oxo-6,9,12,15-tetraoxa-3-azaheptadecan-17-yl) carbamate (15)

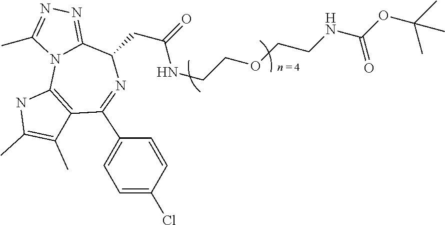

Compound 15 was synthesized from (+)-JQ1 (13.3 mg, 0.029 mmol) and tert-butyl (14-amino-3,6,9,12-tetraoxatetradecyl) carbamate according to General Procedure 3. Obtained 13.0 mg as a clear colorless oil, 62% over two steps.

ESI-MS (m/z): [M+H]+ calcd. for C34H48ClN6O7S+, 719.29; found 719.20.

1H NMR (400 MHZ, CD3OD-d4) δ 7.49-7.39 (m, 4H), 4.63 (dd, J=9.0, 5.2 Hz, 1H), 3.66-3.57 (m, 14H), 3.51-3.41 (m, 5H), 3.33-3.27 (m, 1H) (overlapping CD3OD peak), 3.20 (t, J=5.6 Hz, 2H), 2.70 (s, 3H), 2.45 (s, 3H), 1.70 (s, 3H), 1.42 (s, 9H).

13C NMR (101 MHZ, CD3OD-d4) δ 172.91, 166.13, 157.02, 151.99, 138.13, 137.94, 133.50, 133.20, 132.02, 131.99, 131.36, 129.78, 71.58, 71.55, 71.53, 71.35, 71.25, 71.06, 70.65, 55.19, 41.28, 40.56, 38.75, 28.78, 14.42, 12.93, 11.61. (1 coincident carbonyl peak; 2 coincident aliphatic peaks).

### tert-Butyl(S)-(1-(4-(4-chlorophenyl)-2,3,9-trimethyl-6H-thieno[3,2-f][1,2,4]triazolo[4,3-a][1,4]diazepin-6-yl)-2-oxo-6,9,12,15,18-pentaoxa-3-azaicosan-20-yl) carbamate (16)

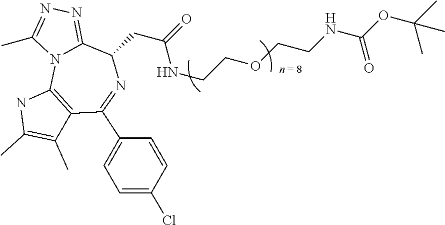

Compound 16 was synthesized from (+)-JQ1 (12.1 mg, 0.027 mmol) and tert-butyl (17-amino-3,6,9,12,15-pentaoxaheptadecyl) carbamate according to General Procedure 3. Obtained 16.8 mg as a clear colorless oil, 83% over two steps.

ESI-MS (m/z): [M+H]+ calcd. for C36H52ClN6O8S+, 763.32; found 763.20.

1H NMR (400 MHZ, CD3OD-d4) δ 7.49-7.39 (m, 4H), 4.63 (dd, J=9.0, 5.1 Hz, 1H), 3.66-3.55 (m, 18H), 3.50-3.42 (m, 5H), 3.35-3.28 (m, 1H) (overlapping CD3OD peak), 3.20 (t, J=5.6 Hz, 2H), 2.70 (s, 3H), 2.45 (s, 3H), 1.70 (s, 3H), 1.42 (s, 9H).

13C NMR (101 MHZ, CD3OD-d4) δ 172.98, 172.90, 166.09, 157.03, 152.13, 138.14, 137.93, 133.50, 133.18, 132.02, 131.98, 131.37, 129.78, 71.61, 71.60, 71.56, 71.55, 71.53, 71.35, 71.25, 71.03, 70.64, 55.20, 41.29, 40.69, 38.80, 28.78, 14.43, 12.94, 11.61. (2 coincident aliphatic peaks).

### tert-Butyl(S)-(1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatricosan-23-yl) carbamate (17)

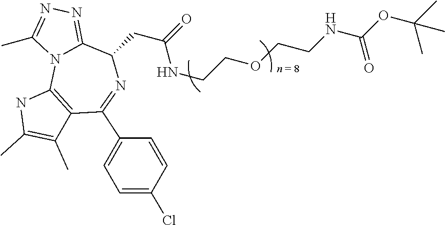

Compound 17 was synthesized from (+)-JQ1 (12.1 mg, 0.027 mmol) and tert-butyl (20-amino-3,6,9,12,15,18-hexaoxaicosyl) carbamate according to General Procedure 3. Obtained 11.6 mg as a clear colorless oil, 54% over two steps.

ESI-MS (m/z): [M+H]+ calcd. for C38H56ClN6O9S+, 807.34; found 807.30.

1H NMR (400 MHZ, CD3OD-d4) δ 7.47-7.39 (m, 4H), 4.62 (dd, J=9.0, 5.1 Hz, 1H), 3.65-3.55 (m, 22H), 3.50-3.41 (m, 5H), 3.32-3.27 (m, 1H) (overlapping CD3OD peak), 3.20 (t, J=5.6 Hz, 2H), 2.69 (s, 3H), 2.44 (s, 3H), 1.70 (s, 3H), 1.42 (s, 9H).

13C NMR (101 MHz, CD3OD-d4) δ 173.00, 172.91, 166.11, 157.03, 152.14, 138.14, 137.94, 133.51, 133.20, 132.03, 131.99, 131.37, 129.79, 71.61, 71.60, 71.56, 71.55, 71.51, 71.35, 71.25, 71.05, 70.64, 55.20, 41.30, 40.57, 38.75, 28.78, 14.42, 12.93, 11.61. (4 coincident aliphatic peaks).

### tert-Butyl(S)-(1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21,24-heptaoxa-3-azahexacosan-26-yl) carbamate (18)

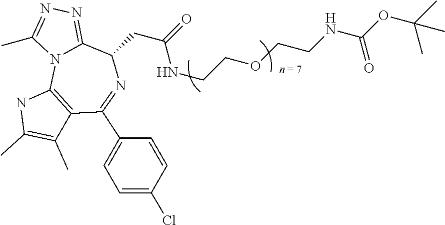

Compound 18 was synthesized from (+)-JQ1 (6.8 mg, 0.015 mmol) and tert-butyl (23-amino-3,6,9,12,15,18,21-heptaoxatricosyl) carbamate according to General Procedure 3. Obtained 12.6 mg as a clear colorless oil, 99% over two steps.

ESI-MS (m/z): [M+H]+ calcd. for C40H60ClN6O10S+, 851.37; found 851.30.

1H NMR (400 MHZ, CD3OD-d4) δ 7.48-7.39 (m, 4H), 4.62 (dd, J=9.1, 5.1 Hz, 1H), 3.65-3.56 (m, 26H), 3.50-3.41 (m, 5H), 3.32-3.27 (m, 1H) (overlapping CD3OD peak), 3.20 (t, J=5.6 Hz, 2H), 2.69 (s, 3H), 2.44 (s, 3H), 1.70 (s, 3H), 1.42 (s, 9H).

13C NMR (101 MHz, CD3OD-d4) δ 172.92, 166.13, 157.04, 152.15, 138.15, 137.95, 133.52, 133.21, 132.03, 132.00, 131.38, 129.80, 71.62, 71.60, 71.56, 71.55, 71.51, 71.36, 71.26, 71.06, 70.64, 55.20, 41.30, 40.58, 38.75, 28.78, 14.42, 12.93, 11.61. (1 coincident carbonyl peak; 5 coincident aliphatic peaks).

### (R)-1-(3-((14-((S)-4-(4-Chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazatetradecyl)oxy)phenyl)-3-(3,4-dimethoxyphenyl) propyl(S)-1-((S)-2-(3,4,5-trimethoxyphenyl) butanoyl) piperidine-2-carboxylate (CEM207, 19)

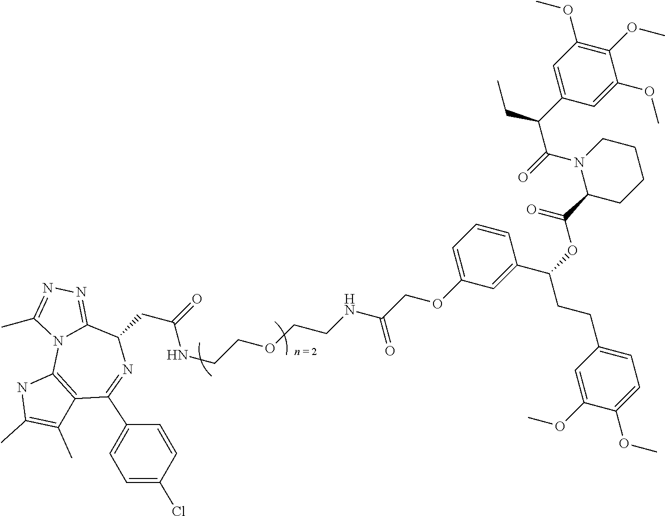

CEM207 (19) was synthesized from compound 13 (6.9 mg, 0.011 mmol) according to General Procedure 4. Obtained 4.0 mg as a white solid, 30% over two steps.

ESI-MS (m/z): [M+H]+ calcd. for C63H77ClN7O13S+, 1206.49; found 1206.30.

1H NMR (400 MHZ, CD3OD-d4) δ 7.47-7.36 (m, 4H), 7.15 (t, J=7.9 Hz, 1H), 6.86-6.80 (m, 2H), 6.78-6.63 (m, 3H), 6.62-6.50 (m, 3H), 5.56 (dd, J=8.3, 5.5 Hz, 1H), 5.37 (d, J=5.5 Hz, 1H), 4.66 (dd, J=8.8, 5.1 Hz, 1H), 4.53-4.45 (m, 2H), 4.07 (d, J=13.4 Hz, 1H), 3.86 (t, J=7.2 Hz, 1H), 3.80 (s, 1H), 3.78 (m, 6H), 3.73 (s, 1H), 3.68-3.65 (m, 7H), 3.62-3.55 (m, 8H), 3.49-3.40 (m, 5H), 3.34-3.28 (m, 1H) (overlapping CD3OD peak), 2.75-2.65 (m, 4H), 2.56-2.47 (m, 1H), 2.46-2.36 (m, 4H), 2.27 (m, 1H), 2.00 (m, 7.7 Hz, 2H), 1.91-1.80 (m, 1H), 1.74-1.56 (m, 7H), 1.54-1.44 (m, 1H), 1.26-1.15 (m, 1H), 0.88 (t, J=7.3 Hz, 3H).

13C NMR (101 MHz, CD3OD-d4) δ 174.96, 172.66, 171.84, 171.09, 166.44, 159.08, 154.57, 150.42, 148.85, 143.59, 138.22, 137.93, 137.72, 136.92, 135.14, 133.63, 133.49, 132.17, 131.95, 131.47, 130.88, 129.83, 121.76, 121.68, 120.65, 115.08, 114.43, 113.66, 113.24, 106.57, 105.99, 77.12, 71.39, 71.34, 70.65, 70.49, 68.31, 61.08, 56.74, 56.55, 56.46, 55.00, 53.61, 51.22, 44.98, 40.53, 39.98, 39.24, 38.47, 32.26, 29.39, 27.61, 26.32, 21.87, 14.42, 12.95, 12.68, 11.57.

### (R)-1-(3-((17-((S)-4-(4-Chlorophenyl)-2,3,9-trimethyl-6H-thieno[3,2-f][1,2,4]triazolo[4,3-a][1,4]diazepin-6-yl)-2,16-dioxo-6,9,12-trioxa-3,15-diazaheptadecyl)oxy)phenyl)-3-(3,4-dimethoxyphenyl) propyl(S)-1-((S)-2-(3,4,5-trimethoxyphenyl) butanoyl) piperidine-2-carboxylate (CEM208, 20)

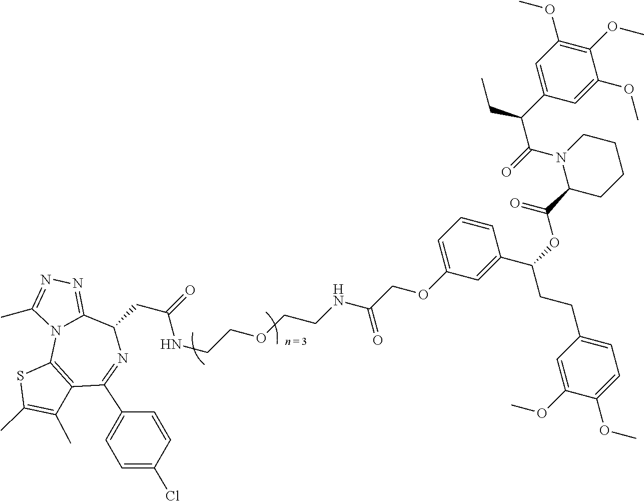

CEM208 (20) was synthesized from compound 14 (5.1 mg, 0.008 mmol) according to General Procedure 4. Obtained 5.9 mg as a white solid, 63% over two steps.

ESI-MS (m/z): [M+H]+ calcd. for C65H81ClN7O14S+, 1250.52; found 1250.40.

1H NMR (400 MHZ, CD3OD-d4) δ 7.48-7.37 (m, 4H), 7.16 (t, J=7.9 Hz, 1H), 6.87-6.81 (m, 2H), 6.79-6.65 (m, 3H), 6.63-6.51 (m, 3H), 5.57 (dd, J=8.3, 5.4 Hz, 1H), 5.38 (d, J=5.4 Hz, 1H), 4.67 (dd, J=8.8, 5.3 Hz, 1H), 4.53-4.45 (m, 2H), 4.08 (d, J=13.2 Hz, 1H), 3.86 (t, J=7.3 Hz, 1H), 3.81 (s, 1H), 3.79 (m, 6H), 3.74 (s, 1H), 3.69-3.65 (m, 7H), 3.63-3.54 (m, 12H), 3.49-3.41 (m, 5H), 3.34-3.28 (m, 1H) (overlapping CD3OD peak), 2.76-2.67 (m, 4H), 2.57-2.48 (m, 1H), 2.46-2.38 (m, 4H), 2.28 (m, 1H), 2.01 (m, 6.9, 6.3 Hz, 2H), 1.93-1.82 (m, 1H), 1.75-1.57 (m, 7H), 1.55-1.45 (m, 1H), 1.27-1.18 (m, 1H), 0.89 (t, J=7.3 Hz, 3H).

13C NMR (101 MHz, CD3OD-d4) δ 174.95, 172.62, 171.85, 171.03, 166.48, 159.09, 154.57, 150.42, 148.85, 143.60, 138.25, 137.93, 137.68, 136.92, 135.13, 133.70, 133.46, 132.21, 131.97, 131.50, 130.89, 129.84, 121.76, 121.69, 120.64, 115.10, 114.44, 113.66, 113.25, 106.57, 105.99, 77.11, 71.61, 71.58, 71.33, 71.29, 70.60, 70.44, 68.29, 61.08, 56.74, 56.55, 56.46, 54.96, 53.61, 51.22, 44.98, 40.56, 40.00, 39.25, 38.42, 32.26, 29.39, 27.61, 26.32, 21.87, 14.43, 12.96, 12.68, 11.57.

### (R)-1-(3-((20-((S)-4-(4-Chlorophenyl)-2,3,9-trimethyl-6H-thieno[3,2-f][1,2,4]triazolo[4,3-a][1,4]diazepin-6-yl)-2,19-dioxo-6,9,12,15-tetraoxa-3,18-diazaicosyl)oxy)phenyl)-3-(3,4-dimethoxyphenyl) propyl(S)-1-((S)-2-(3,4,5-trimethoxyphenyl) butanoyl) piperidine-2-carboxylate (CEM209, 21)

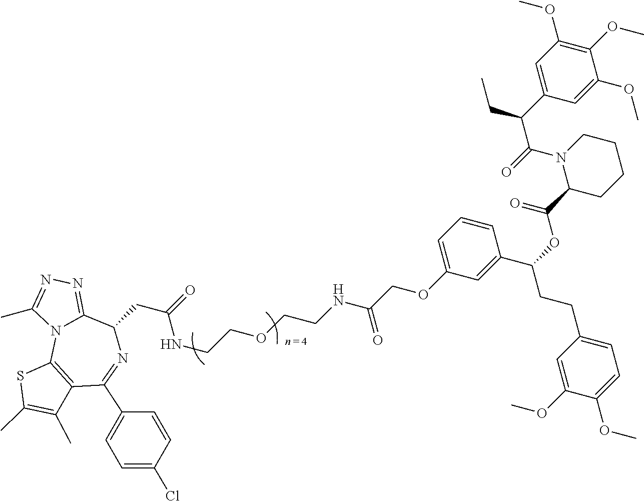

CEM209 (21) was synthesized from compound 15 (6.5 mg, 0.009 mmol) according to General Procedure 4. Obtained 6.6 mg as a white solid, 56% over two steps.

ESI-MS (m/z): [M+H]+ calcd. for C67H85ClN7O15S+, 1294.54; found 1294.40.

1H NMR (400 MHZ, CD3OD-d4) δ 7.48-7.38 (m, 4H), 7.17 (t, J=7.9 Hz, 1H), 6.87-6.82 (m, 2H), 6.79-6.65 (m, 3H), 6.62-6.53 (m, 3H), 5.58 (dd, J=8.3, 5.5 Hz, 1H), 5.38 (d, J=5.4 Hz, 1H), 4.67 (dd, J=7.6, 3.7 Hz, 1H), 4.54-4.45 (m, 2H), 4.08 (d, J=13.7 Hz, 1H), 3.86 (t, J=7.3 Hz, 1H), 3.81 (s, 1H), 3.79 (d??, J=2.6 Hz, 6H), 3.74 (s, 1H), 3.70-3.65 (m, 7H), 3.61 (m, 8H), 3.59-3.52 (m, 8H), 3.49-3.42 (m, 5H), 3.34-3.28 (m, 1H) (overlapping CD3OD peak), 2.76-2.68 (m, 4H), 2.57-2.49 (m, 1H), 2.46-2.37 (m, 4H), 2.28 (m, 1H), 2.07-1.97 (m, 2H), 1.93-1.83 (m, 1H), 1.74-1.57 (m, 7H), 1.55-1.43 (m, 1H), 1.27-1.17 (m, 1H), 0.89 (t, J=7.3 Hz, 3H).

13C NMR (101 MHz, CD3OD-d4) δ 174.95, 172.67, 171.85, 171.01, 166.43, 159.10, 154.57, 150.42, 148.86, 143.61, 138.22, 137.94, 137.73, 136.92, 135.12, 133.65, 133.47, 132.19, 131.98, 131.49, 130.90, 129.84, 121.77, 121.69, 120.64, 115.12, 114.43, 113.66, 113.25, 106.57, 105.99, 77.11, 71.59, 71.58, 71.55, 71.33, 71.31, 70.61, 70.42, 68.29, 61.08, 56.75, 56.56, 56.46, 54.99, 53.60, 51.22, 44.98, 40.58, 40.01, 39.26, 38.46, 32.26, 29.39, 27.62, 26.32, 21.87, 14.43, 12.95, 12.68, 11.57. (1 coincident aliphatic peak).

### (R)-1-(3-((23-((S)-4-(4-Chlorophenyl)-2,3,9-trimethyl-6H-thieno[3,2-f][1,2,4]triazolo[4,3-a][1,4]diazepin-6-yl)-2,22-dioxo-6,9,12,15,18-pentaoxa-3,21-diazatricosyl)oxy)phenyl)-3-(3,4-dimethoxyphenyl) propyl(S)-1-((S)-2-(3,4,5-trimethoxyphenyl) butanoyl) piperidine-2-carboxylate (CEM210, 22)

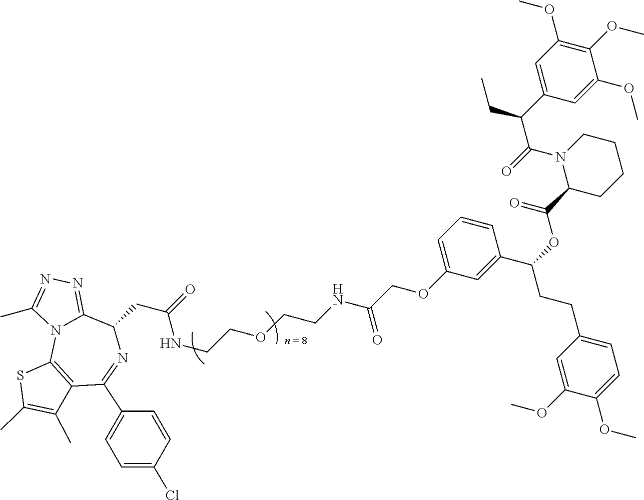

CEM210 (22) was synthesized from compound 16 (4.8 mg, 0.006 mmol) according to General Procedure 4. Obtained 4.8 mg as a white solid, 57% over two steps.

ESI-MS (m/z): [M+H]+ calcd. for C69H89ClN7O16S+, 1338.57; found 1338.30.

1H NMR (400 MHZ, CD3OD-d4) δ 7.49-7.38 (m, 4H), 7.17 (t, J=8.0 Hz, 1H), 6.88-6.82 (m, 2H), 6.80-6.65 (m, 3H), 6.62-6.53 (m, 3H), 5.58 (dd, J=8.2, 5.6 Hz, 1H), 5.39 (d, J=4.9 Hz, 1H), 4.67 (dd, J=8.9, 5.1 Hz, 1H), 4.54-4.45 (m, 2H), 4.08 (d, J=13.4 Hz, 1H), 3.87 (t, J=7.3 Hz, 1H), 3.81 (s, 1H), 3.79 (m, 6H), 3.74 (s, 1H), 3.69-3.66 (m, 7H), 3.63-3.58 (m, 14H), 3.57-3.53 (m, 6H), 3.50-3.42 (m, 5H), 3.34-3.28 (m, 1H) (overlapping CD3OD peak), 2.77-2.67 (m, 4H), 2.58-2.49 (m, 1H), 2.48-2.38 (m, 4H), 2.28 (m, 1H), 2.06-1.98 (m, 2H), 1.95-1.82 (m, 1H), 1.75-1.57 (m, 7H), 1.56-1.47 (m, 1H), 1.27-1.19 (m, 1H), 0.89 (t, J=7.3 Hz, 3H).

13C NMR (101 MHz, CD3OD-d4) δ 174.95, 172.70, 171.85, 171.02, 166.42, 159.10, 154.57, 150.42, 148.85, 143.62, 138.21, 137.94, 137.75, 136.92, 135.12, 133.65, 133.48, 132.19, 131.98, 131.49, 130.90, 129.85, 121.77, 121.69, 120.64, 115.13, 114.43, 113.65, 113.24, 106.56, 105.8. (2 coincident aliphatic peaks).

### (R)-1-(3-((26-((S)-4-(4-Chlorophenyl)-2,3,9-trimethyl-6H-thieno[3,2-f][1,2,4]triazolo[4,3-a][1,4]diazepin-6-yl)-2,25-dioxo-6,9,12,15,18,21-hexaoxa-3,24-diazahexacosyl)oxy)phenyl)-3-(3,4-dimethoxyphenyl) propyl(S)-1-((S)-2-(3,4,5-trimethoxyphenyl) butanoyl) piperidine-2-carboxylate (CEM211, 23)

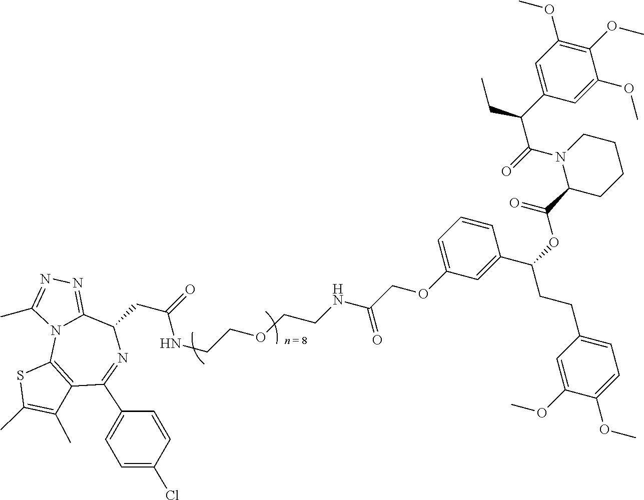

CEM211 (23) was synthesized from compound 17 (8.7 mg, 0.011 mmol) according to General Procedure 4. Obtained 3.1 mg as a white solid, 21% over two steps.

ESI-MS (m/z): [M+H]+ calcd. for C71H93ClN7O17S+, 1382.60; found 1382.40.

1H NMR (400 MHZ, CD3OD-d4) δ 7.48-7.38 (m, 4H), 7.17 (t, J=7.9 Hz, 1H), 6.87-6.82 (m, 2H), 6.80-6.65 (m, 3H), 6.62-6.52 (m, 3H), 5.57 (dd, J=8.3, 5.5 Hz, 1H), 5.38 (d, J=5.5 Hz, 1H), 4.66 (dd, J=8.9, 5.1 Hz, 1H), 4.54-4.45 (m, 2H), 4.07 (d, J=12.7 Hz, 1H), 3.86 (t, J=7.3 Hz, 1H), 3.81 (s, 1H), 3.79 (m, 6H), 3.73 (s, 1H), 3.69-3.66 (m, 7H), 3.63-3.59 (m, 9H), 3.58-3.53 (m, 15H), 3.49-3.41 (m, 5H), 3.33-3.27 (m, 1H) (overlapping CD3OD peak), 2.76-2.67 (m, 4H), 2.56-2.48 (m, 1H), 2.46-2.37 (m, 4H), 2.28 (m, 1H), 2.05-1.97 (m, 2H), 1.94-1.83 (m, 1H), 1.74-1.57 (m, 7H), 1.56-1.46 (m, 1H), 1.26-1.19 (m, 1H), 0.88 (t, J=7.3 Hz, 3H).

13C NMR (101 MHz, CD3OD-d4) δ 176.17, 174.96, 173.59, 171.85, 166.35, 159.11, 154.58, 150.43, 148.86, 143.62, 138.15, 137.94, 137.84, 136.92, 135.13, 133.55, 133.46, 132.15, 132.00, 131.47, 130.91, 129.84, 121.77, 121.69, 120.65, 115.14, 114.44, 113.66, 113.24, 106.57, 105.98, 77.11, 71.60, 71.58, 71.54, 71.53, 71.35, 71.30, 70.63, 70.42, 68.30, 61.09, 56.70, 56.56, 56.46, 55.06, 53.61, 51.23, 44.98, 40.59, 40.03, 39.27, 38.54, 32.26, 29.40, 27.62, 26.33, 21.88, 14.43, 12.95, 12.68, 11.59. (4 coincident aliphatic peaks).

### (R)-1-(3-((29-((S)-4-(4-Chlorophenyl)-2,3,9-trimethyl-6H-thieno[3,2-f][1,2,4]triazolo[4,3-a][1,4]diazepin-6-yl)-2,28-dioxo-6,9,12,15,18,21,24-heptaoxa-3,27-diazanonacosyl)oxy)phenyl)-3-(3,4-dimethoxyphenyl) propyl(S)-1-((S)-2-(3,4,5-trimethoxyphenyl) butanoyl) piperidine-2-carboxylate (CEM212, 24)

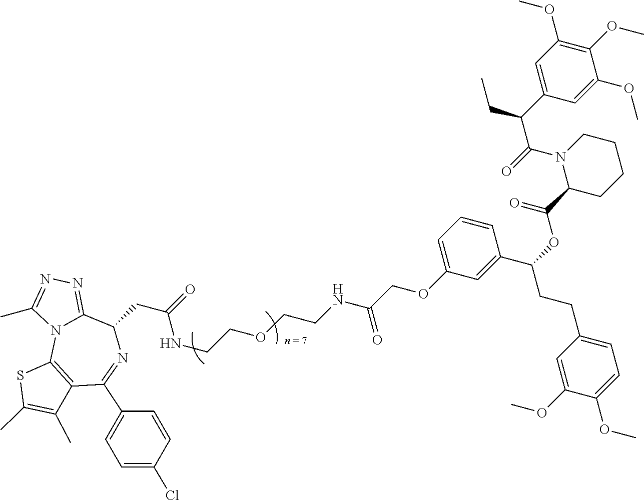

CEM212 (24) was synthesized from compound 18 (5.3 mg, 0.006 mmol) according to General Procedure 4. Obtained 5.3 mg as a white solid, 59% over two steps.

ESI-MS (m/z): [M+H]+ calcd. for C73H97ClN7O18S+, 1426.62; found 714.30 corresponding to [(M+H)/2]+ calcd. for C73H97ClN7O18S, 713.31.

1H NMR (400 MHZ, CD3OD-d4) δ 7.47-7.37 (m, 4H), 7.17 (t, J=8.0 Hz, 1H), 6.88-6.82 (m, 2H), 6.79-6.65 (m, 3H), 6.62-6.52 (m, 3H), 5.58 (dd, J=8.4, 5.4 Hz, 1H), 5.38 (d, J=4.2 Hz, 1H), 4.63 (dd, J=9.1, 5.1 Hz, 1H), 4.54-4.45 (m, 2H), 4.08 (d, J=11.6 Hz, 1H), 3.86 (t, J=7.2 Hz, 1H), 3.81 (s, 1H), 3.79 (m, 6H), 3.73 (s, 1H), 3.69-3.66 (m, 7H), 3.63-3.60 (m, 7H), 3.60-3.53 (m, 21H), 3.48-3.41 (m, 5H), 3.33-3.27 (m, 1H) (overlapping CD3OD peak), 2.76-2.66 (m, 4H), 2.56-2.48 (m, 1H), 2.47-2.38 (m, 4H), 2.28 (m, 1H), 2.05-1.97 (m, 2H), 1.93-1.82 (m, 1H), 1.73-1.58 (m, 7H), 1.54-1.46 (m, 1H), 1.27-1.20 (m, 1H), 0.88 (t, J=7.3 Hz, 3H).

13C NMR (126 MHz, CD3OD-d4) δ 174.96, 172.90, 171.85, 171.05, 166.12, 159.12, 154.58, 150.43, 148.86, 143.62, 138.13, 137.94, 137.52, 136.92, 135.13, 133.52, 133.21, 132.03, 131.99, 131.38, 130.91, 129.79, 121.77, 121.69, 120.65, 115.15, 114.44, 113.67, 113.26, 106.58, 105.99, 77.11, 71.57, 71.55, 71.51, 71.50, 71.34, 71.29, 70.66, 70.45, 68.31, 61.09, 56.75, 56.56, 56.47, 55.20, 53.61, 51.23, 44.98, 40.57, 40.02, 39.27, 38.76, 32.27, 29.40, 27.62, 26.32, 21.87, 14.43, 12.93, 12.68, 11.61. (5 coincident aliphatic peaks).

### Example 3

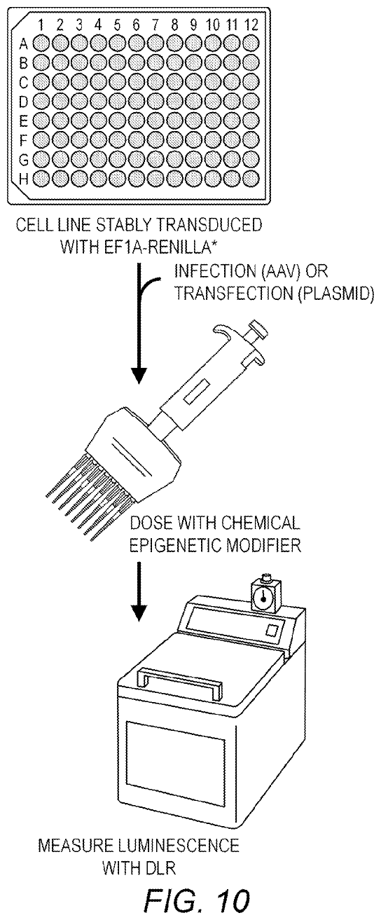

It was investigated whether rAAV transgene expression can be epigenetically controlled. Rapid screening of CEM technology can be performed with dual liucierase assays. (FIG. 10). Briefly, a cell line can be stably transduced with Ef1a-Renilla, enabling normalization to cell number. The cells are then either infected with AAV or tranfected with plasmid and dosed with a chemical epigenetic modifier. Luminescence can then be measured with the Dual Lucieferase Reporter, commercially available from Promega. This approach is described in more detail in the methods section of the example. Gene activation can be described as normalized luciferase or fold change. A measure of total gene expression can be calculated by

$$\frac{Firefly Luc}{Renilla Luc}$$

providing normalized luminescence for each CEM dose used. A measure of dynamic range can be calculated by

$$\frac{Normalized Lum . ( treatment )}{Mean of Normalized Lum . ( no treatment )}$$

providing fold-change from no treatment.

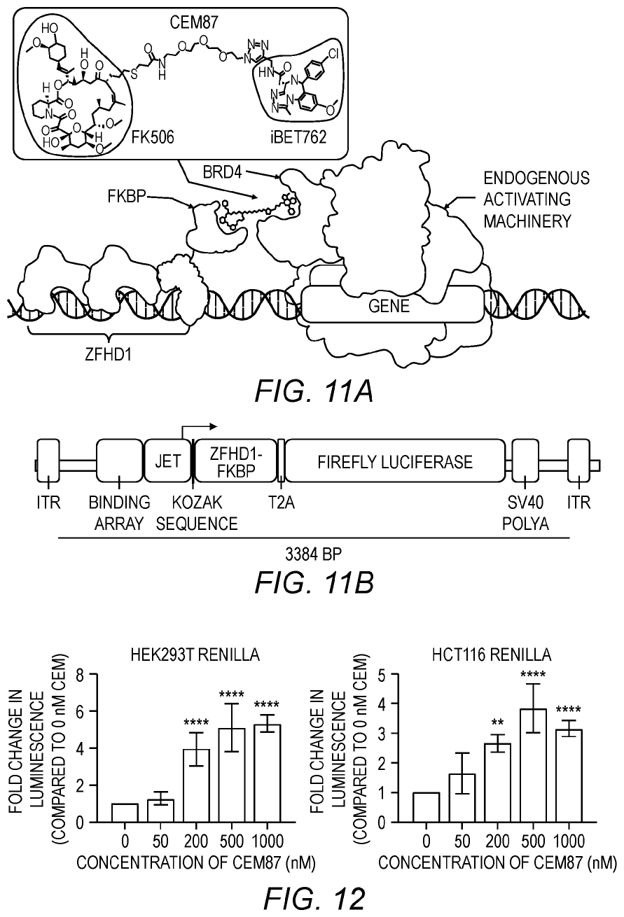

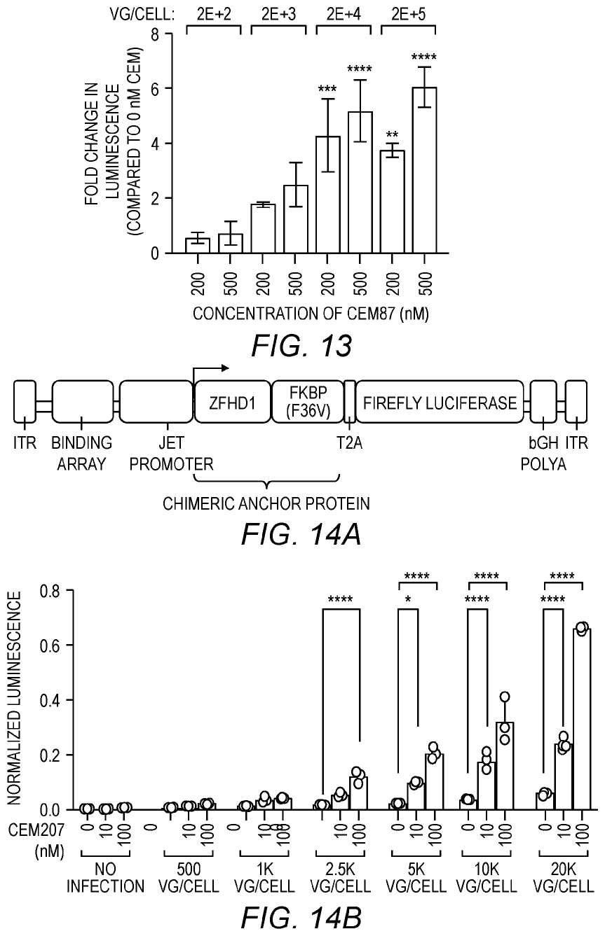

Preliminary regulation of AAV with first generation CEMs is depicted in FIG. 11A. The prototype system is autogenous, saving casssette space. (FIG. 11B). First generation CEM87 shows dose dependent increase in luminscence in HEK293T and HCT116 cells (FIG. 12). Preliminary regulation of AAV with first generation CEMs in HEK293T Renilla cells using the cassette in FIG. 11B results are shown in FIG. 13, with multiplicity of infection (MOI)=number of viral genomes/cell.

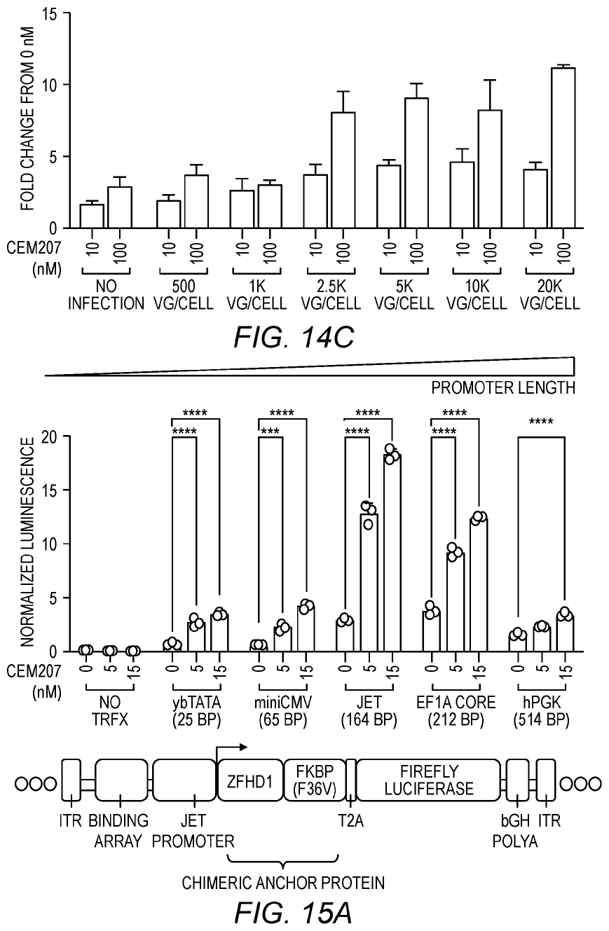

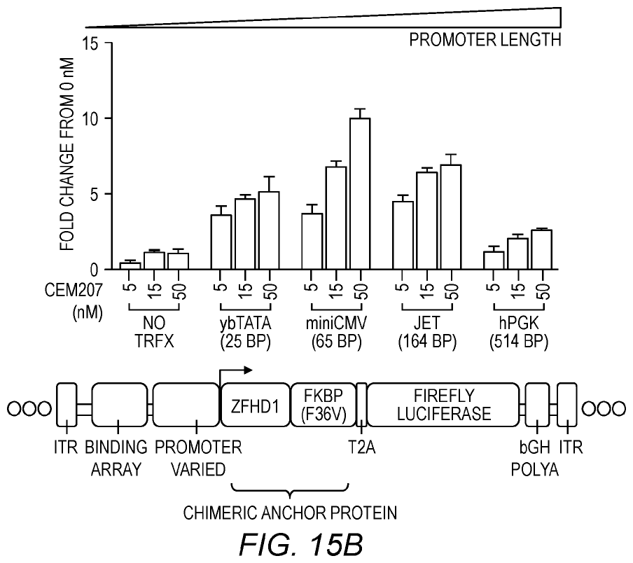

Next, it was determined whether SLF*CEMs can regulate AAV. The cassette shown in FIG. 14A was inserted into rAAV2 and infected into HEK293T Renilla. As shown in FIGS. 14B and 14C, SLF* CEM, CEM207, activation of rAAV2 is compatible with viral titer escalation. Next, it was explored whether the cassette was truly modular by varying the promoter while retaining SLF* CEM successful activation of rAAV2. FIGS. 15A and 15B chart a variety of minimal promoters, including ybTATA, miniCMV, Het, Ef1a core, and hPGK, showing SLF*CEM 207 self-targeting system tolerates a variety of minimal promoters.

As shown in the Examples, the CEM systems of the invention recruit endogenous epigenetic machinery to targeted genetic loci. Both FK506 and SLF*CEM systems can activate AAV expression in a dose dependent manner.

### Methods

### SLF*CEM Multiplicity of Infection

AAV2 Infection with SLF* CEM System Under the Control of JeT, with 0, 10, or 100 nM CEM207.

Day 1 (0 h): 293T cells with stably infected Renilla under the control of full length Ef1a promoter were plated in a 96 well plate; 10 k cells/well, 100 μL media/well. All conditions were plated in biological triplicate.

Day 2 (24 h): Media was replaced dropwise with 100 μL of new media containing the specified MOI of SRW039 AAV2 per well. For the no infection group, media was replaced with new media containing no virus.

Day 3 (48 h): Additional 100 μL of new media containing 2× of the desired final concentration of CEM207 was added to each well dropwise (e.g., for wells with a final concentration of 100 nM, 100 μL of 200 nM CEM207-containing media was added). For the 0 nM triplicates, an additional 100 μL of media containing the vehicle control DMSO was added.

Day 4 (72 h): Data was obtained using the Promega Dula Luciferase Reporter (DLR) assay: first, the cells were washed with 100 μL of PBS/well dropwise followed by 20 μL of 1× passive lysis buffer (PLB) added to each well. The cells were incubated for 15 minutes in PLB and the assay ran on pheraSTAR microplate reader with 100 μL of each DLR reagent, gain=3600.

### SRW039, 066, 067, 054, and 053 Transfection Data

Transfection Evaluating Multiple Promoters—SLF* CEM System with 0, 5, 15 or 50 nM CEM207:

Day 1 (0 h): 293T cells with stably infected Renilla under the control of full length Ef1a promoter were plated in a 96 well plate; 10 k cells/well, 100 μL media/well. All conditions were plated in biological triplicate.

Day 2 (24 h): A PEI transfection mixture of each construct (Jet, ybTATA, miniCMV, Ef1a core, and hPGK) was prepared and added to each well: 0.1 μg of DNA, 0.3 μL of PEI, and 4 μL of Opti-MEM per well, with PEI added last. Reagents were mixed well and incubated for 15 minutes. 4 μL of the appropriate transfection mixture was added to each well.

Day 3 (40 h): Approximately 16 h post transfection, the media in each well was replaced with 100 μL of media containing the appropriate concentration of CEM207 or DMSO as a vehicle control for the 0 nM groups.

Day 4 - - -

Day 5 (96 h): Data obtained using the Promega Dual Luciferase Reporter (DLR) assay: Data was obtained using the Promega Dula Luciferase Reporter (DLR) assay: first, the cells were washed with 100 μL of PBS/well dropwise followed by 20 μL of 1× passive lysis buffer (PLB) added to each well. The cells were incubated for 15 minutes in PLB and the assay ran on pheraSTAR microplate reader with 25 μL of each DLR reagent, gain=2000.

### Example 4. SAHA-Based CEMs

SAHA-based CEMs were prepared, including SAHA ligand, which results in bifunctional molecules having repressor activity. A summary of the molecules prepared is provided below.

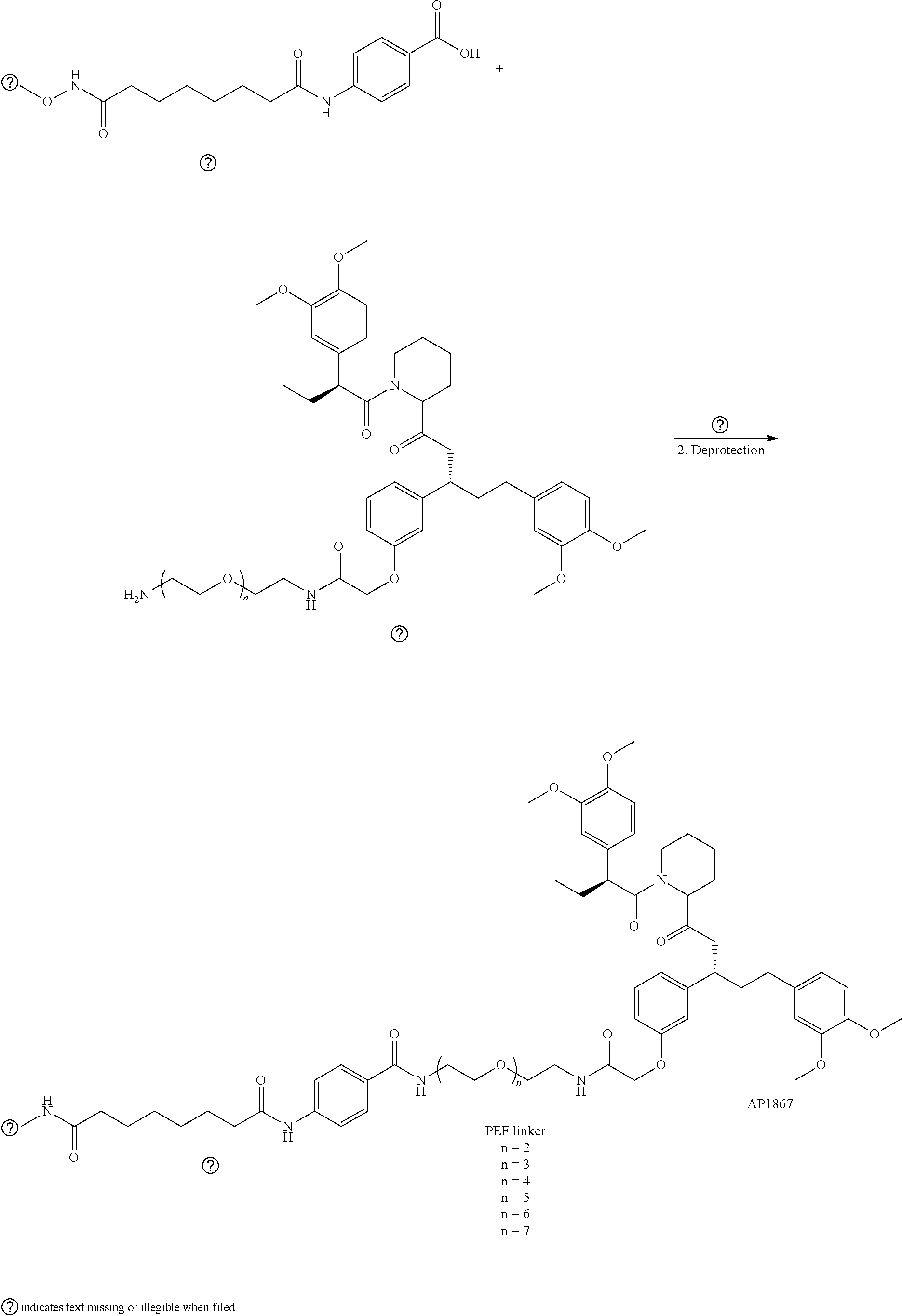

### Synthetic Schemes

### Synthesis of SAHA Analogue (4)

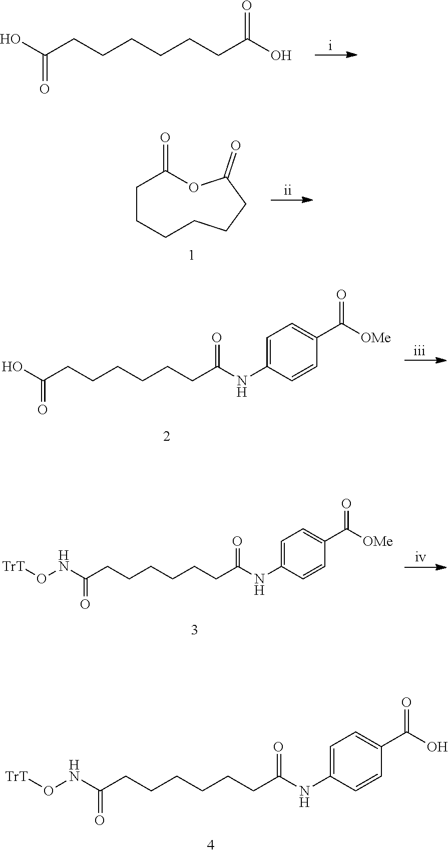

Supplemental scheme 1. Reagents and conditions: i) Acetic anhydride, reflux, 100%; ii) Methyl 4-aminobenzoate in THF, rt, 30%; iii) Ethyl chloroformate, TEA, O-tritylhydroxylamine, and sodium methoxide in THF, rt, 35%; iv) LiOH in THF and water, rt, 100%

### Synthesis of SAHA-Based CEMs, CEM (7)-CEM (8)

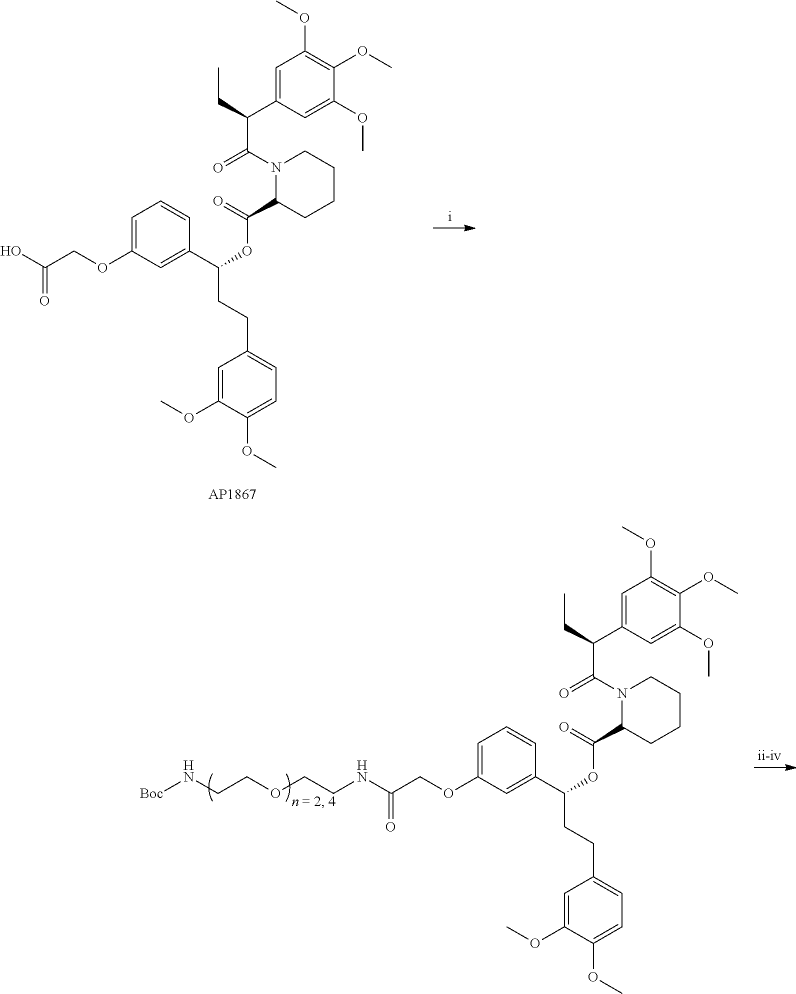

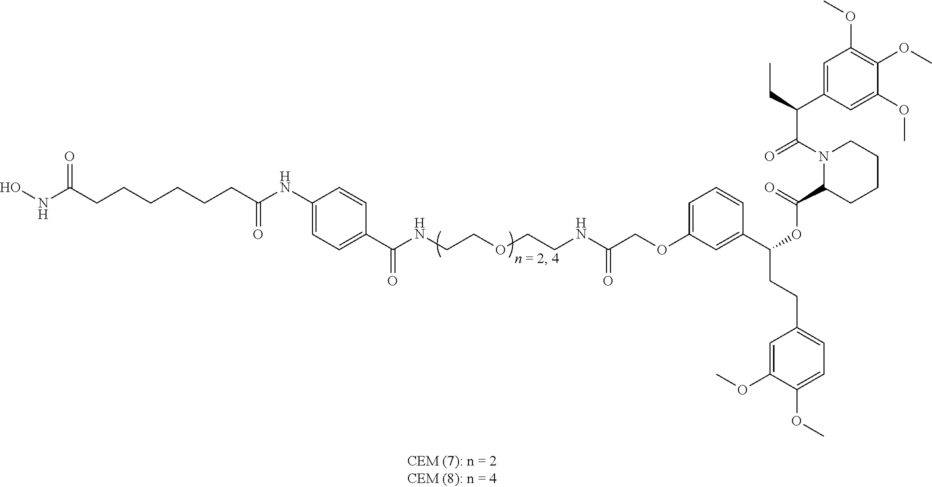

Supplemental scheme 2. Reagents and conditions: i) NH₂-PEGₙ-CH₂CH₂NH-Boc linker with n=2 or 4, TBTU, DIPEA in DMF, rt, 71-75%; ii) TFA 20% (v/v) in DCM, rt; iii) Compound 4, TBTU, DIPEA in DMF, rt; (iv) 5% TIPS/TFA (v/v) in DCM, rt, 50-72% over three steps

General chemistry procedures. Reactions were performed in either round-bottom flasks or glass sample vials under ambient conditions with room temperature (rt) generally 25° C. All reagents and solvents were obtained from commercial suppliers and were used without further purification unless otherwise stated. Specifically, AP1867 were purchased from MedChemExpress. Thin-layer chromatography (TLC) was performed using commercial silica gel 60 F254-coated glass-backed plates. TLC plates were visualized under 254 nm UV light or by immersion in a basic potassium permanganate solution followed by heating with a heat gun for 30 sec.

Normal phase flash column chromatography was performed with a Teledyne Isco CombiFlash®Rf using RediSep®Rf silica columns with the UV detector set to 254 nm and 280 nm. Mobile phases A (DCM) and B (MeOH) were used. Reverse phase column chromatography was performed with a Teledyne Isco CombiFlash®Rf using C18 RediSep®Rf Gold columns with the UV detector set to 220 nm and 254 nm. Mobile phases A (H₂O+0.1% TFA (v/v)) and B (MeOH or ACN) were used. Preparative HPLC was performed using an Agilent Prep 1200 series with the UV detector set to 220 nm and 254 nm. Samples were injected onto a Phenomenex Luna 75×30 mm (5 μm) C18 column. Mobile phases A (H₂O+0.1% TFA) and B (MeOH) were used with a flow rate 30 mL/min.

Analysis of products. Analytical LCMS was used to establish the purity of targeted compounds. All final compounds that were evaluated in biochemical assays had >95% purity as determined by LCMS. Analytical LCMS data for compounds were acquired on an Agilent 6110 Series system with UV detector set to 220 nm, 254 nm, and 280 nm. Samples were injected onto an Agilent ZORBAX Eclipse Plus 4.6×50 mm, 1.8 μm, C18 column at 25° C. Mobile phases A (H₂O+0.1% acetic acid) and B (ACN+1% water and 0.1% acetic acid) were used in a linear gradient from 10% to 100% B in 5 min, followed by a flush at 100% B for another 2 min with a flow rate of 1.0 mL/min. Mass spectra (MS) data were acquired in positive ion mode using an Agilent 6110 single quadrupole mass spectrometer with an electrospray ionization (ESI) source. All ¹H and ¹³C nuclear magnetic resonance (NMR) spectra were recorded in deuterated solvent (CD3OD-d₄ or DMSO-d₆) on a Varian 400 MR NMR spectrometer at 400 MHZ. All 13C NMR spectra were recorded in deuterated solvent (CD₃OD-d₄ or DMSO-d₆) on a Varian 400 MR NMR or a Varian Inova 500 NMR spectrometer at 101 MHz and 126 MHZ, respectively. Chemical shifts are reported in parts per million (ppm) and are referenced to residual un-deuterated solvent (CD₃OD-d₄ referenced at 3.31 ppm for ¹H NMR and 49.00 ppm for ¹³C NMR; or DMSO-d₆ referenced at 2.50 ppm for ¹H NMR and 39.52 ppm for ¹³C NMR). Coupling constants are reported in Hertz (Hz) and peaks multiplicities as either a singlet(s), doublet (d), triplet (t), q (quartet), quint (quintet), m (multiplet), dd (doublet of doublets), dt (doublet of triplets), td (triplet of doublets), or br (broad singlet).

Chemistry abbreviations. Methanol (MeOH), acetonitrile (ACN), dichloromethane (DCM), ethyl acetate (EtOAc), dimethyl sulfoxide (DMSO), N,N-dimethylformamide (DMF), trifluoroacetic acid (TFA), 2-(1H-benzo[d][1,2,3]triazol-1-yl)-1,1,3,3-tetramethyluronium tetrafluoroborate (TBTU), N-ethyl-N-isopropylpropan-2-amine (DIPEA).

### Synthetic Procedures

General Procedure 1 (Scheme S2, Step i): Amidation of AP1867 with NH₂-PEGₙ Linkers

To a solution of AP1867 (1.0 equiv.) in DMF (0.025 molar) was added TBTU (1.2 equiv.) and DIPEA (1.5 equiv.). After 5-10 min, a solution of NH₂-PEGₙ-CH₂CH₂NH-Boc (1.1 equiv.) with n=2 or 4 in DMF (0.025 molar) was added to the initial reaction mixture, and the reaction was left to stir at rt. After 1 h, the reaction was concentrated under reduced pressure and purified by preparative HPLC (10-100%, MeOH in water containing 0.1% TFA) to yield the desired product, compound 5 or 6, as a clear colorless oil.

General Procedure 2 (Scheme S2, Steps ii-iv): Amidation of Compounds 5-6 with Compound 4

Compounds 5-6 were N-Boc deprotected in 20% (v/v) TFA in DCM (2.0 mL) at rt and concentrated under reduced pressure. Each deprotected intermediate (1.0 equiv.) was then re-dissolved in DMF (0.025 molar) and DIPEA (1.5 equiv.) to neutralize any remaining excess TFA.

In a separate flask, compound 4 (1.0 equiv.) was dissolved in DMF (0.025 molar) and pre-activated with TBTU (1.2 equiv.) and DIPEA (1.5 equiv.); after 5-10 min, the solution was added to the initial reaction mixture containing the deprotected amine intermediate, and the reaction was left to stir at rt. After 1 h, the reaction was concentrated under reduced pressure and purified by preparative HPLC (10-100%, MeOH in water containing 0.1% TFA) to yield the final intermediate.

The final intermediate was subsequently dissolved in DCM (2 mL) and deprotected with 5% TIPS/TFA (v/v) at rt. The reaction was concentrated under reduced pressure and purified by preparative HPLC (10-100%, MeOH in water containing 0.1% TFA) to yield the desired product, compound (CEM (7) or CEM (8)), as a white solid following lyophilization.

Oxonane-2,9-dione (1)

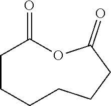 [SMILES: O=C1CCCCCCC(=O)O1]

To a flask was added octanedioic acid (1000 mg, 5.74 mmol, 1.0 equiv.) and acetic anhydride (1.00 mL, 10.6 mmol, 1.85 equiv.). The reaction was heated to reflux overnight. After 16 h, the reaction was concentrated under reduced pressure to yield the desired product, compound 1, as a white solid (897 mg, 100%).

ESI-MS (m/z): [M+H]⁺ calcd. for C₈H₁₃O₃⁺, 157.08; mass not observed likely due to poor ionization.

¹H NMR (400 MHZ, DMSO-d₆) δ 2.51 (t, J=7.2 Hz, 4H), 1.61-1.49 (m, 4H), 1.36-1.27 (m, 4H).

¹³C NMR (101 MHz, DMSO-d₆) δ 174.51, 172.05, 33.63, 28.30, 24.39, 21.08.

### 8-((4-(Methoxycarbonyl)phenyl)amino)-8-oxooctanoic acid (2)

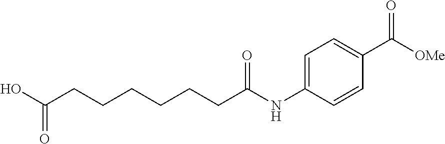 [SMILES: COC(=O)c1ccc(NC(=O)CCCCCCC(=O)O)cc1]

To a solution of compound 1 (100 mg, 0.640 mmol, 1.0 equiv.) in THF (2.0 mL) was added methyl 4-aminobenzoate (96.8 mg, 0.640 mmol, 1.0 equiv.). The reaction was left to stir at rt overnight. After 16 h, the reaction was quenched by the addition of water and extracted with DCM 3×. The combined organic layers were then washed with brine, dried over Na₂SO₄, filtered, and concentrated under reduced pressure. The resulting crude product was purified via flash chromatography (0-20%, MeOH in DCM) to yield the desired product, compound 2, as a clear colorless oil 8-((4-(methoxycarbonyl)phenyl)amino)-8-oxooctanoic acid (59.0 mg, 30%).

ESI-MS (m/z): [M+H]+ calcd. for C₁₆H₂₂NO₅⁺, 308.14; found 308.10.

¹H NMR (400 MHZ, CD₃OD-d₄) δ 7.95 (d, J=8.8 Hz, 2H), 7.68 (d, J=8.8 Hz, 2H), 3.87 (s, 3H), 2.40 (t, J=7.5 Hz, 2H), 2.29 (dt, J=7.4, 2.7 Hz, 3H), 1.74-1.67 (m, 2H), 1.66-1.57 (m, 3H), 1.41-1.38 (m, 2H).

¹³C NMR (101 MHZ, CD3OD-d4) δ 177.61, 174.88, 168.17, 144.58, 131.51, 126.21, 120.11, 52.46, 37.98, 34.86, 29.97, 29.91, 26.57, 25.91.

### Methyl 4-(8-oxo-8-((trityloxy)amino) octanamido)benzoate (3)

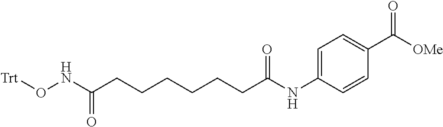

To a solution of compound 2 (59.0 mg, 0.192 mmol, 1.0 equiv.) in THF (1.6 mL) under nitrogen was added ethyl chloroformate (23.9 μL, 0.250 mmol, 1.3 equiv.) and TEA (37.5 μL, 0.269 mmol, 1.4 equiv.). The reaction was left to stir at rt. After 30 min, O-tritylhydroxylamine (89.9 mg, 0.326 mmol, 1.7 equiv.) and sodium methoxide (17.6 mg, 0.326 mmol, 1.7 equiv.) were added. The reaction was left to stir at rt. After 16 h, the reaction was quenched by the addition of water and extracted with EtOAc 3×. The combined organic layers were then washed with brine, dried over Na₂SO₄, filtered, and concentrated under reduced pressure. The resulting crude product was purified via flash chromatography (0-100%, EtOAc in hexanes) to yield the desired product, compound 3, as a clear colorless oil (38.0 mg, 35%).

ESI-MS (m/z): [M+H]⁺ calcd. for C₃₅H₃₇N₂O₅+, 565.26; found 587.20 corresponding to [M+Na]⁺ (calcd. for C₃₅H₃₆N₂O₅Na⁺, 587.25).

¹H NMR (400 MHZ, DMSO-d₆) δ 10.19 (s, 1H), 10.15 (NH, 1H), 7.92-7.87 (m, 2H), 7.73 (d, J=8.8 Hz, 2H), 7.36-7.24 (m, 15H), 3.81 (s, 3H), 2.29 (t, J=7.4 Hz, 2H), 1.80-1.71 (m, 2H), 1.50 (apparent p, J=7.5 Hz, 2H), 1.24-0.96 (m, 6H).

¹³C NMR (101 MHZ, DMSO-d6) δ 171.85, 170.29, 165.82, 143.72, 142.47, 130.24, 128.95, 127.48, 127.38, 123.62, 118.32, 51.84, 36.46, 31.97, 28.31, 28.14, 24.78, 24.66.

### 4-(8-Oxo-8-((trityloxy)amino) octanamido)benzoic acid (4)

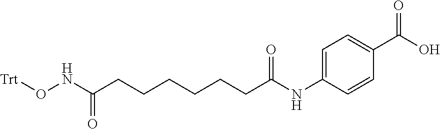

To a solution of compound 3 (38.0 mg, 0.067 mmol, 1.0 equiv.) in THF (1.0 mL) was added LiOH (3.22 mg, 0.135 mmol, 2.0 equiv.). A few drops of water were added to dissolve the LiOH. The reaction was left to stir at rt. After 16 h, the reaction was quenched by the addition of water. The pH was adjusted to ˜pH 4 using 1 M HCl. The reaction was extracted with EtOAc 3×. The combined organic layers were then washed with brine, dried over Na₂SO₄, filtered, and concentrated under reduced pressure to yield the desired product, compound 4, as a clear colorless oil (37.1 mg, 100%).

ESI-MS (m/z): [M+H]⁺ calculated for C₃₄H₃₅N₂O₅+, 551.25; found 573.20 corresponding to [M+Na]⁺ (calcd. for C₃₅H₃₆N₂O₅Na⁺, 573.24).

¹H NMR (400 MHZ, DMSO-d₆) δ 10.20-10.11 (m, 2H), 7.87 (d, J=8.7 Hz, 2H), 7.69 (d, J=8.8 Hz, 2H), 7.34-7.27 (m, 15H), 2.28 (t, J=7.4 Hz, 2H), 1.81-1.69 (m, 2H), 1.49 (apparent p, J=7.5 Hz, 2H), 1.21-0.95 (m, 6H).

¹³C NMR (101 MHz, DMSO-d6) δ 171.77, 170.29, 166.98, 143.27, 142.47, 130.33, 128.95, 127.50, 127.39, 125.05, 118.20, 36.46, 31.97, 28.32, 28.15, 24.82, 24.67.

### (R)-3-(3,4-dimethoxyphenyl)-1-(3-((2,2-dimethyl-4,15-dioxo-3,8,11-trioxa-5,14-diazahexadecan-16-yl)oxy)phenyl) propyl (R)-1-((S)-2-(3,4,5-trimethoxyphenyl) butanoyl) piperidine-2-carboxylate (5)

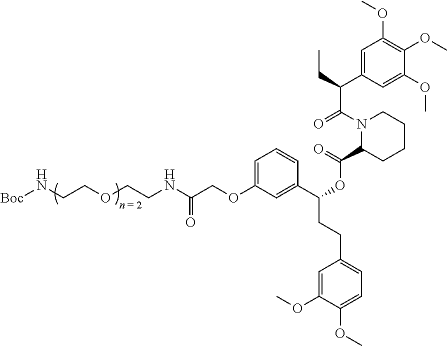

Compound 5 was synthesized from AP1867 (9.61 mg, 0.014 mmol) and tert-butyl (2-(2-(2-aminoethoxy) ethoxy)ethyl) carbamate according to General Procedure 1. Obtained 9.60 mg as a clear colorless oil, 75%.

ESI-MS (m/z): [M+H]⁺ calculated for C₄₉H₇₀N₃O₁₄, 924.48; found 924.30.

¹H NMR (400 MHZ, CD₃OD-d₄) δ 7.19 (t, J=7.9 Hz, 1H), 6.89-6.84 (m, 2H), 6.80-6.66 (m, 3H), 6.62-6.54 (m, 3H), 5.59 (dd, J=8.3, 5.5 Hz, 1H), 5.40-5.37 (m, 1H), 4.56-4.47 (m, 2H), 4.12-4.05 (m, 1H), 3.89-3.84 (m, 1H), 3.83-3.79 (m, 7H), 3.75 (apparent s, 1H), 3.71-3.66 (m, 7H), 3.59-3.54 (m, 6H), 3.50-3.45 (m, 4H), 3.19 (t, J=5.6 Hz, 2H), 2.72 (td, J=13.3, 3.0 Hz, 1H), 2.59-2.50 (m, 1H), 2.48-2.39 (m, 1H), 2.32-2.24 (m, 1H), 2.14-1.97 (m, 2H), 1.95-1.84 (m, 1H), 1.74-1.47 (m, 5H), 1.42 (s, 9H), 1.30-1.21 (m, 1H), 0.90 (t, J=7.3 Hz, 3H).

¹³C NMR (126 MHZ, CD₃OD-d₄) δ δ 174.97, 171.86, 171.42, 171.11, 159.14, 154.57, 150.43, 148.87, 143.61, 137.94, 136.92, 135.14, 130.90, 121.78, 120.71, 115.17, 114.39, 113.66, 113.26, 106.58, 77.13, 71.28, 71.07, 70.47, 68.32, 61.09, 56.74, 56.57, 56.55, 56.46, 53.62, 51.24, 44.98, 41.25, 39.96, 39.23, 32.26, 29.39, 28.77, 27.61, 26.32, 21.86, 12.67. (2 coincident aliphatic peaks)

### (R)-3-(3,4-dimethoxyphenyl)-1-(3-((2,2-dimethyl-4,21-dioxo-3,8,11,14,17-pentaoxa-5,20-diazadocosan-22-yl)oxy)phenyl) propyl (R)-1-((S)-2-(3,4,5-trimethoxyphenyl) butanoyl) piperidine-2-carboxylate (6)

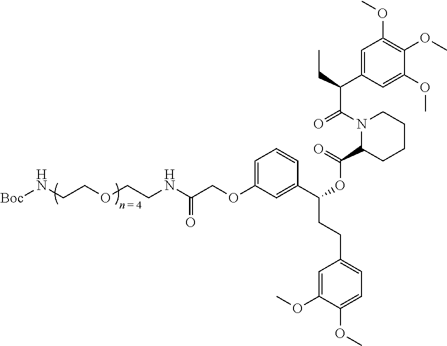

Compound 6 was synthesized from Ap1867 (9.06 mg, 0.013 mmol) and tert-butyl (14-amino-3,6,9,12-tetraoxatetradecyl) carbamate (according to General Procedure 1. Obtained 9.40 mg as a clear colorless oil, 71%.

ESI-MS (m/z): [M+H]⁺ calculated for C53H78N3016, 1012.53; found 912.40.

¹H NMR (400 MHZ, CD₃OD-d₄) δ δ 7.19 (t, J=8.0 Hz, 1H), 6.89-6.84 (m, 2H), 6.81-6.66 (m, 3H), 6.63-6.55 (m, 3H), 5.59 (dd, J=8.2, 5.6 Hz, 1H), 5.40-5.37 (m, 1H), 4.55-4.47 (m, 2H), 4.12-4.05 (m, 1H), 3.89-3.84 (m, 1H), 3.82-3.82 (m, 1H), 3.81-3.79 (m, 6H), 3.75 (apparent s, 1H), 3.70-3.67 (m, 7H), 3.62-3.56 (m, 14H), 3.47 (t, J=5.5 Hz, 4H), 3.19 (t, J=5.6 Hz, 2H), 2.72 (td, J=13.4, 3.1 Hz, 1H), 2.59-2.50 (m, 1H), 2.49-2.39 (m, 1H), 2.32-2.25 (m, 1H), 2.10-1.96 (m, 2H), 1.94-1.84 (m, 1H), 1.75-1.49 (m, 5H), 1.42 (s, 9H), 1.29-1.22 (m, 1H), 0.90 (t, J=7.3 Hz, 3H).

¹³C NMR (126 MHZ, CD₃OD-d₄) δ δ 174.97, 171.86, 171.42, 171.06, 159.13, 154.58, 150.44, 148.87, 143.62, 137.95, 136.92, 135.13, 130.91, 121.78, 120.69, 115.16, 114.43, 113.67, 113.27, 106.58, 77.12, 71.55, 71.53, 71.31, 71.24, 71.05, 70.43, 68.31, 61.09, 56.74, 56.57, 56.55, 56.46, 53.61, 51.24, 44.98, 40.03, 39.25, 32.26, 29.39, 28.78, 27.62, 26.32, 21.86, 12.67. (3 coincident aliphatic peaks)

### (R)-3-(3,4-dimethoxyphenyl)-1-(3-((1-(4-(8-(hydroxyamino)-8-oxooctanamido)phenyl)-1,12-dioxo-5,8-dioxa-2,11-diazatridecan-13-yl)oxy)phenyl) propyl (R)-1-((S)-2-(3,4,5-trimethoxyphenyl) butanoyl) piperidine-2-carboxylate (CEM7)

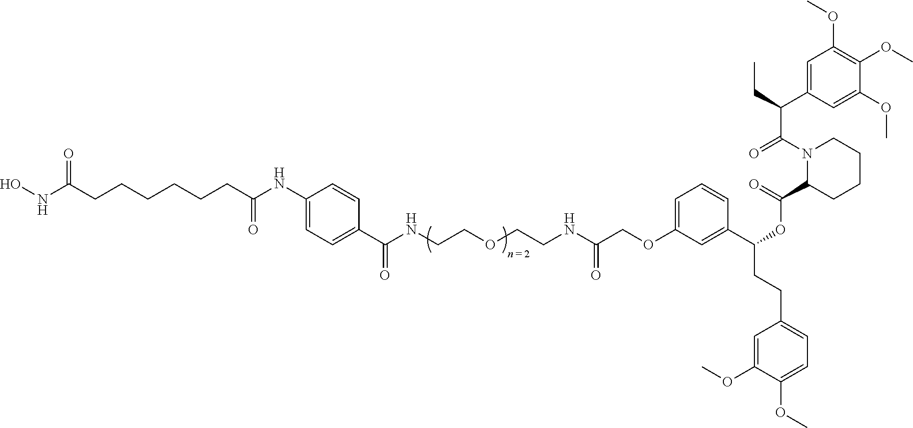

CEM (7) was synthesized from compound 5 (9.6 mg, 0.010 mmol) according to General Procedure 2. Obtained 5.82 mg as a white solid following lyophilization, 50% over three steps.

ESI-MS (m/z): [M+H]⁺ calcd. for C₅₉H₈₀N₅O₁₆⁺, 1114.55; found 1114.40.

¹H NMR (400 MHZ, CD₃OD-d₄) δ 7.77 (d, J=8.7 Hz, 2H), 7.65 (d, J=8.8 Hz, 2H), 7.17 (t, J=7.9 Hz, 1H), 6.87-6.81 (m, 2H), 6.80-6.64 (m, 3H), 6.62-6.53 (m, 3H), 5.57 (dd, J=8.2, 5.4 Hz, 1H), 5.38 (d, J=5.4 Hz, 1H), 4.54-4.45 (m, 2H), 4.11-4.04 (m, 1H), 3.89-3.84 (m, 1H), 3.81-3.78 (m, 7H), 3.74 (s, 1H), 3.70-3.65 (m, 7H), 3.64-3.52 (m, 10H), 3.47-3.42 (m, 2H), 2.72 (td, J=13.4, 3.0 Hz, 1H), 2.63-2.42 (m, 2H), 2.37 (t, J=7.5 Hz, 2H), 2.31-2.23 (m, 1H), 2.09 (t, J=7.4 Hz, 2H), 2.05-1.96 (m, 2H), 1.92-1.82 (m, 1H), 1.77-1.56 (m, 8H), 1.55-1.46 (m, 1H), 1.43-1.35 (m, 4H), 1.29-1.19 (m, 1H), 0.89 (t, J=7.3 Hz, 3H).

¹³C NMR (126 MHz, CD3OD-d4) δ 175.16, 174.99, 174.74, 171.87, 171.10, 169.58, 159.11, 154.56, 150.40, 148.83, 143.59, 143.20, 137.90, 136.92, 135.13, 130.89, 130.50, 129.20, 121.77, 120.68, 120.22, 115.10, 114.42, 113.64, 113.24, 106.56, 77.13, 71.33, 71.30, 70.63, 70.46, 68.29, 61.09, 56.74, 56.55, 56.45, 53.62, 51.60, 51.22, 49.85, 44.98, 40.87, 39.94, 39.20, 37.94, 33.69, 32.24, 29.91, 29.83, 29.38, 27.61, 26.58, 26.31, 21.85, 12.67.

### (R)-3-(3,4-dimethoxyphenyl)-1-(3-((1-(4-(8-(hydroxyamino)-8-oxooctanamido)phenyl)-1,18-dioxo-5,8,11,14-tetraoxa-2,17-diazanonadecan-19-yl)oxy)phenyl) propyl (R)-1-((S)-2-(3,4,5-trimethoxyphenyl) butanoyl) piperidine-2-carboxylate (CEM (8))

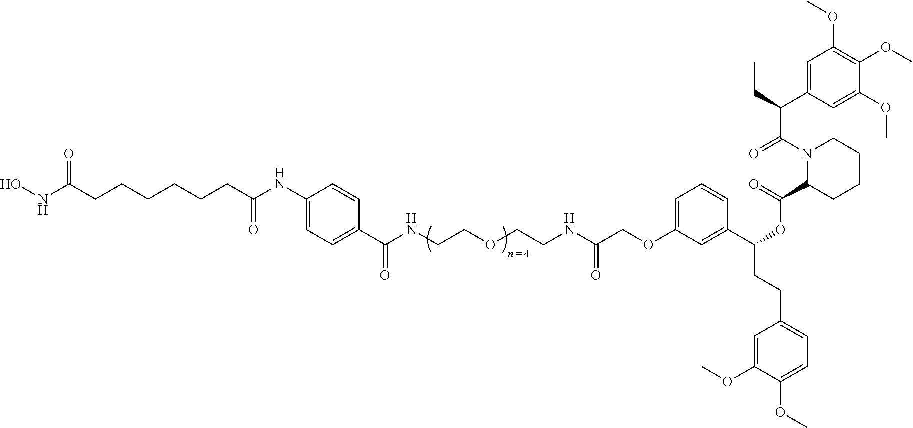

CEM (8) was synthesized from compound 6 (9.6 mg, 0.093 mmol) according to General Procedure 2. Obtained 7.97 mg as a white solid following lyophilization, 72% over three steps.

ESI-MS (m/z): [M+H]⁺ calcd. for C₆₃H₈₈N₅O₁₈⁺, 1202.60; found 1202.50.

¹H NMR (400 MHZ, CD₃OD-d₄) δ 7.79 (d, J=8.6 Hz, 2H), 7.66 (d, J=8.6 Hz, 2H), 7.18 (t, J=7.9 Hz, 1H), 6.87-6.82 (m, 2H), 6.79-6.65 (m, 3H), 6.62-6.54 (m, 3H), 5.58 (dd, J=8.1, 5.5 Hz, 1H), 5.40-5.36 (m, 1H), 4.54-4.47 (m, 2H), 4.12-4.05 (m, 1H), 3.89-3.84 (m, 1H), 3.83-3.76 (m, 7H), 3.74 (s, 1H), 3.70-3.65 (m, 7H), 3.64-3.50 (m, 18H), 3.47-3.42 (m, 2H), 2.76-2.68 (m, 1H), 2.57-2.45 (m, 2H), 2.38 (t, J=7.4 Hz, 2H), 2.30-2.25 (m, 1H), 2.09 (t, J=7.4 Hz, 2H), 2.04-1.98 (m, 2H), 1.93-1.85 (m, 1H), 1.74-1.59 (m, 8H), 1.54-1.47 (m, 1H), 1.43-1.35 (m, 4H), 1.28-1.21 (m, 1H), 0.89 (t, J=7.3 Hz, 3H).

¹³C NMR (126 MHZ, CD₃OD-d₄) δ 175.15, 174.97, 174.74, 171.85, 171.05, 169.57, 159.10, 154.56, 150.40, 148.84, 143.61, 143.21, 137.91, 136.92, 135.12, 130.90, 130.52, 129.24, 121.77, 120.66, 120.21, 115.12, 114.43, 113.65, 113.24, 106.56, 77.11, 71.52, 71.51, 71.50, 71.29, 71.27, 70.60, 70.43, 68.29, 61.09, 56.74, 56.55, 56.46, 53.61, 51.60, 51.22, 49.85, 44.97, 40.94, 40.01, 39.24, 37.94, 33.69, 32.25, 29.91, 29.82, 29.38, 27.62, 26.58, 26.31, 21.86, 12.68. (1 coincident or missing carbonyl peak; 1 coincident aliphatic peak).

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A chemical epigenetic modifier (CEM) of Formula I: 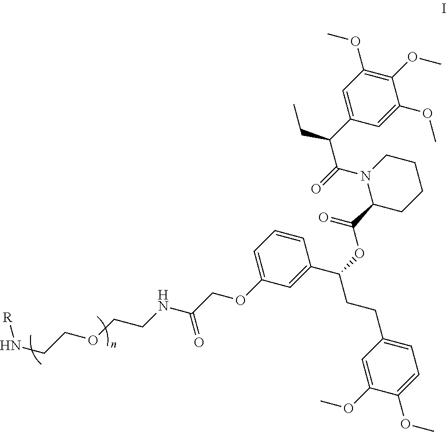
or a pharmaceutically acceptable salt thereof, wherein:
n=1-10; and
R is a chromatin regulatory protein ligand.

2. The CEM of claim 1, having the Formula Ia: 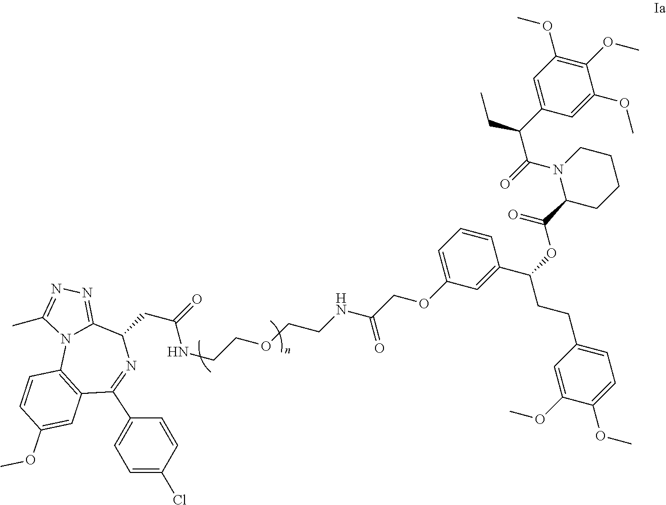
or a pharmaceutically acceptable salt thereof.

3. The CEM of claim 1, having the Formula Ib: 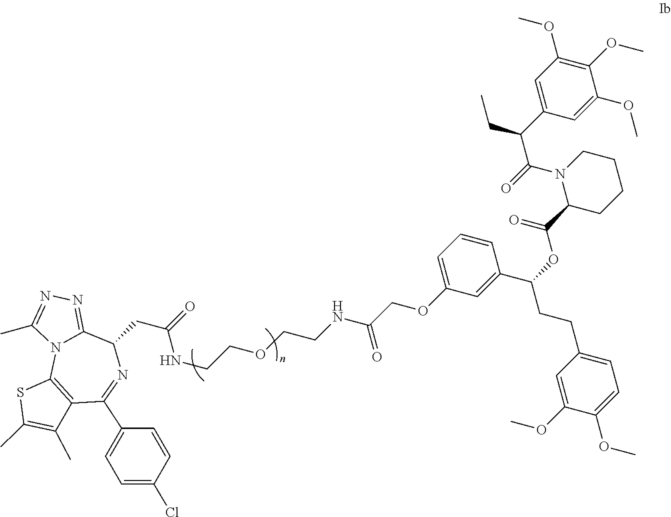
or a pharmaceutically acceptable salt thereof.

4. The CEM of claim 1, having the Formula Ic: 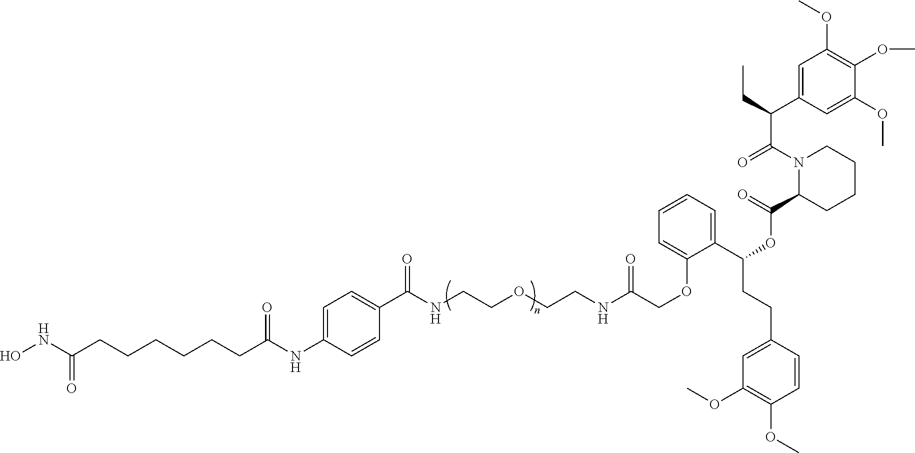
or a pharmaceutically acceptable salt thereof.

5. The CEM of claim 1, wherein n=2-7.

6. A composition comprising the CEM of claim 1 and a FK506-binding protein (FKBP) with a F36V mutation.

7. The composition of claim 6, wherein the FKBP with a F36V mutation is a fusion protein and further comprises a guide RNA (gRNA) binding polypeptide.

8. The composition of claim 7, wherein the gRNA binding polypeptide is a MS2 polypeptide.

9. The composition of claim 7 further comprising a gRNA comprising i) a gene targeting polynucleotide sequence and ii) a polynucleotide sequence that is recognized by the gRNA binding polypeptide.

10. The composition of claim 9, wherein the polynucleotide sequence recognized by the gRNA binding polypeptide of the gRNA forms a MS2 stem-loop motif.

11. The composition of claim 9, further comprising a CRISPR-Cas protein capable of forming a complex with the gRNA.

12. The composition of claim 11, wherein the CRISPR-Cas protein is a Cas9, Cas12a or Cas13 protein.

13. The composition of claim 11, wherein the CRISPR-Cas protein is a deactivated Cas9 protein (dCas9).

14. The composition of claim 6 further comprising a protein with a sequence-specific DNA-binding domain, wherein the DNA binding domain comprises one of a zinc finger DNA binding domain, a helix-loop-helix DNA binding domain, a bZIP DNA binding domain, an HMG-box DNA binding domain, a transcription activator-like effector DNA binding domain, a transcription factor DNA binding domain, or a restriction endonuclease DNA binding domain.

15. A composition comprising a transgene delivery vector comprising a polynucleotide comprising a nucleic acid binding domain recognition sequence and a transgene expression cassette, and a fusion protein comprising a nucleic acid binding domain that binds to the recognition sequence fused to a domain that binds to the chemical epigenetic modifier of claim 1.

16. A pharmaceutical composition comprising the CEM of claim 1 and a pharmaceutically acceptable carrier.

17-18. (canceled)

19. A method of modulating expression of a target gene in a subject, the method comprising:
administering to the subject:
1) a fusion protein comprising a FK506-binding protein (FKBP) polypeptide with a F36V mutation that binds a CEM and a gRNA binding polypeptide that binds gRNA;
2) a gRNA comprising a gene targeting polynucleotide sequence that binds to a target gene sequence and polynucleotide sequence recognized by the gRNA binding polypeptide;
3) a protein with a DNA binding domain that binds to the target gene sequence and the gRNA gene targeting polynucleotide sequence; and
4) the CEM of claim 1, thereby modulating expression of the target gene.

20. A method of treating a disorder that is treatable by modulating expression of a gene in a subject in need thereof, the method comprising:
administering to the subject:
1) a fusion protein comprising a FK506-binding protein (FKBP) polypeptide with a F36V mutation that binds a CEM and a gRNA binding polypeptide that binds gRNA;
2) a gRNA comprising a gene targeting polynucleotide sequence that binds to a target gene sequence and a polynucleotide sequence recognized by the gRNA binding polypeptide;
3) a protein with a DNA binding domain that binds to the target gene sequence and the gRNA targeting polynucleotide sequence; and
4) the CEM of claim 1, thereby treating the disorder.

21-27. (canceled)

28. A method of modulating expression of a transgene from a transgene delivery vector in a subject, the method comprising:
administering to the subject a transgene delivery vector comprising a polynucleotide comprising a transgene expression cassette and a nucleic acid binding domain recognition sequence;
administering to the subject a fusion protein comprising a nucleic acid binding domain that binds to the recognition sequence fused to a domain that binds a CEM of claim 1; and
administering to the subject the CEM of claim 1; thereby modulating expression of the transgene.

29. A method of treating a disorder that is treatable by expression of a transgene from a transgene delivery vector in a subject in need thereof, the method comprising:
administering to the subject a transgene delivery vector comprising a polynucleotide comprising a transgene expression cassette and a nucleic acid binding domain recognition sequence;
administering to the subject a fusion protein comprising a nucleic acid binding domain that binds to the recognition sequence fused to a domain that binds a CEM of claim 1; and
administering to the subject the CEM of claim 1;
thereby treating the disorder.

30-39. (canceled)
